SEQUENCE LISTING (14 sequences)

SEQ ID NO: 1 - protein, 4 aa, synthetic construct
GFLG

SEQ ID NO: 2 - protein, 6 aa, synthetic construct
GILGVP

SEQ ID NO: 3 - protein, 10 aa, synthetic construct
  SITE 1: note = Pyroglutamic acid
EHWSYGLRPG

SEQ ID NO: 4 - protein, 9 aa, synthetic construct
  DISULFID 1..9: note = cyclic via cysteine cysteine disulfide bond (Cys-Cys)
CGNKRTRGC

SEQ ID NO: 5 - protein, 4 aa, synthetic construct
KKKK

SEQ ID NO: 6 - protein, 5 aa, synthetic construct
KKKKK

SEQ ID NO: 7 - protein, 6 aa, synthetic construct
KKKKKK

SEQ ID NO: 8 - protein, 4 aa, synthetic construct
GGGG

SEQ ID NO: 9 - protein, 4 aa, synthetic construct
GLFG

SEQ ID NO: 10 - protein, 6 aa, synthetic construct
PVGLIG

SEQ ID NO: 11 - protein, 10 aa, synthetic construct
  SITE 10: note = Pyroglutamic acid
GPRLGYSWHE

SEQ ID NO: 12 - protein, 5 aa, synthetic construct
GLFGK

SEQ ID NO: 13 - protein, 4 aa, synthetic construct
GGGK

SEQ ID NO: 14 - protein, 7 aa, synthetic construct
PVGLIGK

(19) United States
(12) Patent Application Publication
(10) Pub. No.: US 2024/0335548 A1
(43) Pub. Date: Oct. 10, 2024

(54) MACROMOLECULES

(71) Applicant: Starpharma Pty Ltd, Abbotsford (AU)

(72) Inventors: David Owen, Preston (AU); Brian Devlin Kelly, Abbotsford (AU); Peter Karellas, Abbotsford (AU)

(21) Appl. No.: 18/507,435

(22) Filed: Nov. 13, 2023

Related U.S. Application Data
(63) Continuation of application No. 16/925,464, filed on Jul. 10, 2020, now abandoned.
Application No. 16/925,464 is a continuation of application No. 16/039,997, filed on Jul. 19, 2018, now abandoned.
Application No. 16/039,997 is a continuation-in-part of application No. 15/688,780, filed on Aug. 28, 2017, now Pat. No. 10,265,409.
Application No. 15/688,780 is a continuation of application No. 14/124,651, filed on Jan. 14, 2014, now Pat. No. 9,744,246.
(60) Provisional application No. 61/493,886, filed on Jun. 6, 2011.

(51) Int. Cl. A61K 47/59; A61K 47/60; A61K 47/64; A61K 47/65; A61K 47/68; C08G 69/10; C08G 69/40; C08G 69/48; C08G 83/00

(52) U.S. Cl. CPC A61K 47/59; A61K 47/60; A61K 47/645; A61K 47/65; A61K 47/68; A61K 47/6885; C08G 69/10; C08G 69/40; C08G 69/48; C08G 83/003; C08G 83/004

(57) ABSTRACT

A macromolecule includes i) a dendrimer comprising a core and at least one generation of building units, the outermost generation of building units having surface amino groups wherein at least two different terminal groups are covalently attached to the surface amino groups of the dendrimer, ii) a first terminal group which is a residue of a pharmaceutically active agent comprising a hydroxyl group, and iii) a second terminal group which is a pharmacokinetic modifying agent. The pharmaceutically active agent is cabazitaxel. The first terminal group is covalently attached to the surface amino group of the dendrimer through a diacid linker, the diacid linker comprising an alkyl chain interrupted by one or more oxygen, sulfur or nitrogen atoms, or a pharmaceutically acceptable salt thereof.

### REFERENCE TO RELATED APPLICATIONS

This application is a continuation application of U.S. patent application Ser. No. 16/925,464, filed on Jul. 10, 2020, which is a continuation of U.S. Ser. No. 16/039,997, filed on Jul. 19, 2018, now abandoned, which is a continuation-in-part of U.S. Ser. No. 15/688,780, filed on Aug. 28, 2017, now U.S. Pat. No. 10,265,409, issued on Apr. 23, 2019, which is a continuation of U.S. Ser. No. 14/124,651, filed on Jan. 14, 2014, now issued as U.S. Pat. No. 9,744,246, issued on Aug. 29, 2017, which is a U.S. national stage filing under 35 U.S.C. 371 (c) based on International Patent Application No. PCT/AU2012/000647, filed on Jun. 6, 2012, which claims priority to U.S. Provisional Application No. 61/493,886, filed on Jun. 6, 2011, the entire contents of each of the above referenced applications.

### REFERENCE TO SEQUENCE LISTING

The present application contains a Sequence Listing XML file which has been submitted electronically in XML format and is hereby incorporated by reference in its entirety. Said XML copy, Jan. 22, 2024, is named 2024_01_22_136080-01403_SL.xml, and is 14,226 bytes in size.

### FIELD OF THE INVENTION

The present invention relates to a macromolecule comprising a dendrimer having surface amine groups to which at least two different terminal groups are attached including a pharmaceutically active agent and a pharmacokinetic modifying agent, the pharmaceutically active agent being attached covalently through a diacid linker. Pharmaceutical compositions and methods of treatment are also described.

### BACKGROUND OF THE INVENTION

There are a number of difficulties associated with the formulation and delivery of pharmaceutically active agents including poor aqueous solubility, toxicity, low bioavailability, instability under biological conditions, lack of targeting to the site of action and rapid in vivo degradation.

To combat some of these difficulties, pharmaceutically active agents may be formulated with solubilising agents which themselves may cause side effects such as hypersensitivity and may require premedication to reduce these side effects. Alternative approaches include encapsulation of the pharmaceutically active agent in liposomes, micelles or polymer matrices or attachment of the pharmaceutically active agent to liposomes, micelles and polymer matrices.

Although these approaches may improve some of the problems associated with the formulation and delivery of pharmaceutically active agents, many still have drawbacks.

Oncology drugs can be particularly difficult to formulate and have side effects that may limit the dosage amount and regimen that can be used for treatment. This can result in reduced efficacy of the treatment. For example, taxane drugs such as paclitaxel, docetaxel and cabazitaxel have low aqueous solubility and are often formulated with solubilisation excipients such as polyethoxylated caster oils (Cremophor EL) or polysorbate 80. Although these solubilisation excipients allow increased amounts of drug in the formulation, they are known to result in significant side effects themselves including hypersensitivity. To reduce hypersensitivity, premedication with steroids such as dexamethasone is sometimes used in the dosage regimen. However, this also has drawbacks as corticosteroids have side effects and are not able to be used in diabetic patients, which form a significant subset of patients over 50 with breast cancer.

The use of liposomes, micelles and polymer matrices as carriers either encapsulating or having the pharmaceutical agent attached, while allowing solubilisation of the pharmaceutically active agent and in some cases improved bioavailability and targeting, present difficulties in relation to release of the pharmaceutically active agent. In some cases, the carrier degrades rapidly releasing the pharmaceutically active agent before it has reached the target organ. In other cases, the release of the pharmaceutically active agent from the carrier is variable and therefore may not reach a therapeutic dose of drug in the body or in the target organ.

Another difficulty with liposome, micelle and polymer matrices as carriers is that drug loading can be variable. This can result in some batches of a particular composition being effective while others are not and/or difficulties in registration of a product for clinical use because of variability in the product.

In addition these molecules may be unstable or poorly characterised materials, may suffer from polydispersity, and due to their nature be difficult to analyse and characterise. They may also have difficult routes of manufacture. These difficulties, especially with regard to analysis and batch to batch inconsistency, significantly impede the path to regulatory submission and approval.

With pharmaceutically active agents that have poor aqueous solubility, often the delivery method is limited, for example, to parenteral administration. This may limit the dosage regimen available and the dosage that may be delivered.

There is a need for alternative formulations and delivery means for delivering drugs to reduce side effects, improve dosage regimens and improve the therapeutic window which may lead to improvements in compliance and efficacy of the drug in patients.

### SUMMARY OF THE INVENTION

The invention is predicated in part on the discovery that macromolecules comprising a dendrimer with surface amino groups having at least two different terminal groups attached to the surface amino groups of the dendrimer and wherein the first terminal group is a pharmaceutically active agent covalently attached to the surface amino group through a diacid linker and the second terminal group is a pharmacokinetic modifying agent may allow high drug loading, improved solubility and controlled release of the pharmaceutically active agent.

In a first aspect of the invention there is provided a macromolecule comprising:
i) a dendrimer comprising a core and at least one generation of building units, the outermost generation of building units having surface amino groups, wherein at least two different terminal groups are covalently attached to the surface amino groups of the dendrimer;
ii) a first terminal group which is a residue of a pharmaceutically active agent comprising a hydroxyl group;
iii) a second terminal group which is a pharmacokinetic modifying agent;
   wherein the first terminal group is covalently attached to the surface amino group of the dendrimer through a diacid linker, or a pharmaceutically acceptable salt thereof.

In some embodiments the pharmaceutically active agent is an oncology drug, especially docetaxel, paclitaxel, cabazitaxel, camptothecin, topotecan, irinotecan or gemcitabine. In other embodiments the pharmaceutically active agent is a steroid, especially testosterone. In some embodiments, the pharmaceutically active agent is a sparingly soluble or insoluble in aqueous solution.

In some embodiments the pharmacokinetic modifying agent is polyethylene glycol, especially polyethylene glycol having a molecular weight in the range of 220 to 2500 Da, more especially 570 to 2500 Da. In some embodiments, the polyethylene glycol has a molecular weight between 220 and 1100 Da, especially 570 and 1100 Da. In other embodiments, the polyethylene glycol has a molecular weight between 1000 and 5500 Da or 1000 and 2500 Da, especially 1000 and 2300 Da.

In some embodiments the diacid linker has the formula:

—C(O)-J-C(O)—X—C(O)—

wherein X is selected from —C₁-C₁₀alkylene-, —(CH₂)ₛ-A-(CH₂)ₜ— and Q; —C(O)-J- is absent, an amino acid residue or a peptide of 2 to 10 amino acid residues, wherein the —C(O)— is derived from the carboxy terminal of the amino acid or peptide;
A is selected from —O—, —S—, —NR₁—, —N⁺(R₁)₂—, —S—S—, —[OCH₂CH₂], —O—, —Y—, and —O—Y—O—;
Q is selected from Y or —Z═N—NH—S(O)_{w}—Y—;
Y is selected from cycloalkyl, heterocycloalkyl, aryl and heteroaryl;
Z is selected from —(CH₂)ₓ—C(CH₃)═, —(CH₂)ₓCH═, cycloalkyl and heterocycloalkyl;
R₁ is selected from hydrogen and C₁-C₄ alkyl;
s and t are independently selected from 1 and 2;
r is selected from 1, 2 and 3;
w is selected from 0, 1 and 2; and
x is selected from 1, 2, 3 and 4.

In some embodiments the dendrimer has 1 to 8 generations of building units, especially 3 to 6 generations of building units. In some embodiments the dendrimer is a dendrimer comprising building units of lysine or lysine analogues. In other embodiments the dendrimer comprises building units of polyetherhydroxylamine.

In some embodiments the first terminal group and the second terminal group are present in a 1:1 ratio. In some embodiments the macromolecule comprises a third terminal group which is a blocking group, especially an acyl group such as acetate. In some embodiments the ratio of the first terminal group, second terminal group and third terminal group is 1:2:1.

In some embodiments, at least 50% of the terminal groups comprise a first or second terminal group.

In some embodiments the dendrimer comprises a targeting agent attached to a functional group on the core optionally through a spacer group, especially where the targeting agent is selected from luteinising hormone releasing hormone, a luteinising hormone releasing hormone analog such as deslorelin, LYP-1 and an antibody or fragment thereof.

In some embodiments the macromolecule has a particulate size of less than 1000 nm, especially between 5 and 1000 nm, more especially between 5 and 400 nm, most especially between 5 and 50 nm. In some embodiments, the macromolecule has a molecular weight of at least 30 kDa, especially 40 to 300 kDa, more especially 40 to 150 kDa.

In another aspect of the invention there is provided a macromolecule comprising:
i) a dendrimer comprising a core and at least one generation of building units, the outermost generation of building units having surface amino groups wherein at least two different terminal groups are covalently attached to the surface amino groups of the dendrimer;
ii) a first terminal group which is a residue of a pharmaceutically active agent comprising a hydroxyl group; and
iii) a second terminal group which is a pharmacokinetic modifying agent;
   wherein the pharmaceutically active agent is cabazitaxel;
   and wherein the first terminal group is covalently attached to the surface amino group of the dendrimer through a diacid linker, the diacid linker comprising an alkyl chain interrupted by one or more oxygen, sulfur or nitrogen atoms, or a pharmaceutically acceptable salt thereof.

In some embodiments, the core is covalently attached to building units via amide linakges, each amide linkage being formed between a nitrogen atom present in the core unit and the carbon atom of an acyl group present in a building unit.

In some embodiments the diacid linker has the formula:

—C(O)—X—C(O)—

wherein X is —(CH₂)ₛ-A-(CH₂)ₜ—; A is —O—, —S— or —NR₁—; R₁ is selected from hydrogen and C₁-C₄ alkyl; and s and t are independently selected from 1 and 2. In some embodiments X is —CH₂-A-CH₂—. In some embodiments the diacid linker is —C(O)—CH₂OCH₂—C(O)—.

In some embodiments the pharmacokinetic modifying agent comprises polyethylene glycol (PEG). In some embodiments the polyethylene glycol has a molecular weight in the range of 1000 to 2500 Da.

In some embodiments the dendrimer has 4 to 6 generations of building units. In some embodiments the dendrimer has 5 generations of building units. In some embodiments the dendrimer is a dendrimer comprising building units of lysine having the structure:

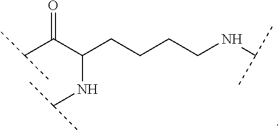

Other examples of suitable building units include:

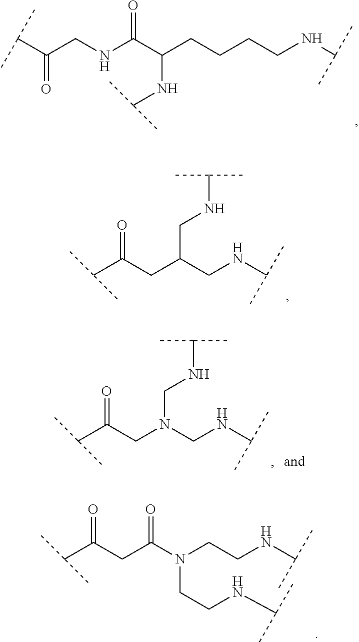

In some embodiments the core is a benzhydrylyamide of lysine (BHALys).

In some embodiments at least 75% of the terminal groups comprise one of the first or second terminal groups. In some embodiments a pharmaceutically active agent is bound to greater than 44% of the total number of surface amine groups. In some embodiments a pharmacokinetic modifying agent is bound to greater than 46% of the total number of surface amine groups. In some embodiments the first terminal group and the second terminal group are present in about a 1:1 ratio.

In another aspect of the invention there is provided a pharmaceutical composition comprising the macromolecule of the invention and a pharmaceutically acceptable carrier. In some embodiments, the composition is substantially free of solubilisation excipients such as polyethoxylated caster oils (eg: Cremphor EL) and polysorbate 80. By removing the solubilisation excipient the composition of dendrimer is less likely to cause side effects such as acute or delayed hypersensitivity including life-threatening anaphylaxis and/or severe fluid retention.

In some embodiments the composition is formulated for parenteral delivery.

In some embodiments the macromolecule is formulated as a slow-release formulation. In some embodiments the linker selected to allow controlled-release of pharmaceutically active agent. In some embodiments, the macromolecule is formulated to release greater than 50% of the pharmaceutically active agent in between 5 minutes to 60 minutes. In other embodiments, the macromolecule is formulated to release greater than 50% of the pharmaceutically active agent in between 2 hours and 48 hours. In yet other embodiments, the macromolecule is formulated to release greater than 50% of the pharmaceutically active agent in between 5 days and 30 days.

In another aspect of the invention there is provided a method of treating or suppressing the growth of a cancer comprising administering an effective amount of a macromolecule or pharmaceutical composition of the invention in which the pharmaceutically active agent of the first terminal group is an oncology drug.

In another aspect of the invention there is provided a method of treating or suppressing the growth of a cancer comprising administering an effective amount of a macromolecule according to some embodiments in which the pharmaceutically active agent is cabazitaxel.

In some embodiments, the tumors are primary or metastatic tumors of the prostate, testes, lung, colon, pancreas, kidney, bone, spleen, brain, head and/or neck, breast, gastrointestinal tract, skin or ovary. In some embodiments the cancer is prostate cancer or breast cancer.

In some embodiments, the method comprises administration of a composition of a macromolecule that is substantially free of polyethoxylated caster oils such as Cremophor® EL, or Kolliphor®, or polysorbate 80.

In another aspect of the invention there is provided a method of reducing hypersensitivity upon treatment with an oncology drug comprising administering a pharmaceutical composition of the present invention, wherein the composition is substantially free from solubilisation excipients such as Cremophor EL and polysorbate 80.

In a further aspect of the invention there is provided a method of reducing the toxicity of an oncology drug or formulation of an oncology drug, comprising administering a macromolecule of the invention in which the oncology drug is the pharmaceutically active agent of the first terminal group.

In some embodiments, the toxicity that is reduced is hematologic toxicity, neurological toxicity, gastrointestinal toxicity, cardiotoxicity, hepatotoxicity, nephrotoxicity, ototoxicity or encephalotoxicity.

In yet a further aspect of the invention there is provided a method of reducing side effects associated with an oncology drug or formulation of an oncology drug, comprising administering a macromolecule of the invention in which the oncology drug is the pharmaceutically active agent of the first terminal group.

In some embodiments, the side effects which are reduced are selected from neutropenia, leukopenia, thrombocytopenia, myelotoxicity, myelosuppression, neuropathy, fatigue, non-specific neurocognitive problems, vertigo, encephalopathy, anemia, dysgeusia, dyspnea, constipation, anorexia, nail disorders, fluid retention, asthenia, pain, nausea, vomiting mucositis, alopecia, skin reactions, myalgia, hypersensitivity and anaphylaxis.

In some embodiments, the need for premedication with agents such as corticosteroids and anti-histamines is reduced or eliminated.

In yet another aspect of the invention there is provided a method of treating or preventing a disease or disorder related to low testosterone levels comprising administering a macromolecule or pharmaceutical composition of the invention in which the pharmaceutically active agent is testosterone.

In some embodiments, the composition is formulated for transdermal delivery, especially by transdermal patch optionally having microneedles.

In some embodiments, there is provided a method of reducing the toxicity of, or reducing side effects associated with, cabazitaxel, or formulation of cabazitaxel, or of reducing hypersensitivity in a subject upon treatment with cabazitaxel or a formulation of cabazitaxel, comprising administering a macromolecule according to some embodiments in which the pharmaceutically active agent is cabazitaxel.

### BRIEF DESCRIPTION OF THE DRAWINGS

FIG. 1 shows the efficacy of a compound (SPL9048) according to some embodiments and a comparator compound (cabazitaxel) in mice represented by change in mean tumour volume (TV) (mm³) over time in a breast cancer tumour model study.

FIG. 2 shows the mean change in body weight in mice following administration of a compound (SPL9048) according to some embodiments and a comparator compound (cabazitaxel) over time in a breast cancer model study.

FIG. 3 shows the efficacy of compounds (SPL8996, SPL9005 and SPL9006) according to some embodiments and a comparator compound (cabazitaxel) in mice represented by change in mean tumour volume (TV) (mm³) over time in a breast cancer tumour model study.

FIG. 4 shows the mean change in body weight in mice following administration of compounds (SPL8996, SPL9005, and SPL9006) according to some embodiments and a comparator compound (cabazitaxel) over time in a breast cancer tumour model study.

FIGS. 5 and 6 show the results of a neutropenia toxicity study data for both male and female rats, following administration of a compound (SPL9048) according to some embodiments and a comparator compound (cabazitaxel/Jevtana®).

### DESCRIPTION OF THE INVENTION

A singular forms “a”, “an” and “the” include plural aspects unless the context clearly indicates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein, the term “alkyl” refers to a straight chain or branched saturated hydrocarbon group having 1 to 10 carbon atoms. Where appropriate, the alkyl group may have a specified number of carbon atoms, for example, C1-4alkyl which includes alkyl groups having 1, 2, 3 or 4 carbon atoms in a linear or branched arrangement. Examples of suitable alkyl groups include, but are not limited to, methyl, ethyl, n-propyl, i-propyl, n-butyl, i-butyl, t-butyl, n-pentyl, 2-methylbutyl, 3-methylbutyl, 4-methylbutyl, n-hexyl, 2-methylpentyl, 3-methylpentyl, 4-methylpentyl, 5-methylpentyl, 2-ethylbutyl, 3-ethylbutyl, heptyl, octyl, nonyl and decyl.

The term “alkylene” as used herein refers to a straight-chain divalent alkyl group having 1 to 10 carbon atoms. Where appropriate, the alkylene group may have a specified number of carbon atoms, for example C₁-C₆ alkylene includes-CH₂—, —(CH₂)₂—, —(CH₂)₃—, —(CH₂)₄—, —(CH₂)₅ and —(CH₂)₆—.

As used herein, the term “cycloalkyl” refers to a saturated or unsaturated cyclic hydrocarbon. The cycloalkyl ring may include a specified number of carbon atoms. For example, a 3 to 8 membered cycloalkyl group includes 3, 4, 5, 6, 7 or 8 carbon atoms. Examples of suitable cycloalkyl groups include, but are not limited to, cyclopropyl, cyclobutyl, cyclopentanyl, cyclopentenyl, cyclohexanyl, cyclohexenyl, 1,4-cyclohexadienyl, cycloheptanyl and cyclooctanyl.

As used herein, the term “aryl” is intended to mean any stable, monocyclic or bicyclic carbon ring of up to 7 atoms in each ring, wherein at least one ring is aromatic. Examples of such aryl groups include, but are not limited to, phenyl, naphthyl, tetrahydronaphthyl, indanyl, biphenyl and binaphthyl.

The term “heterocycloalkyl” or “heterocyclyl” as used herein, refers to a cyclic hydrocarbon in which one to four carbon atoms have been replaced by heteroatoms independently selected from the group consisting of N, N(R), S, S(O), S(O)2 and O. A heterocyclic ring may be saturated or unsaturated. Examples of suitable heterocyclyl groups include tetrahydrofuranyl, tetrahydrothiophenyl, pyrrolidinyl, pyrrolinyl, pyranyl, piperidinyl, pyrazolinyl, dithiolyl, oxathiolyl, dioxanyl, dioxinyl, morpholino and oxazinyl.

The term “heteroaryl” as used herein, represents a stable monocyclic or bicyclic ring of up to 7 atoms in each ring, wherein at least one ring is aromatic and at least one ring contains from 1 to 4 heteroatoms selected from the group consisting of O, N and S. Heteroaryl groups within the scope of this definition include, but are not limited to, acridinyl, carbazolyl, cinnolinyl, quinoxalinyl, quinazolinyl, pyrazolyl, indolyl, benzotriazolyl, furanyl, thienyl, thiophenyl, 3,4-propylenedioxythiophenyl, benzothienyl, benzofuranyl, benzodioxane, benzodioxin, quinolinyl, isoquinolinyl, oxazolyl, isoxazolyl, imidazolyl, pyrazinyl, pyridazinyl, pyridinyl, pyrimidinyl, pyrrolyl, tetrahydroquinoline, thiazolyl, isothiazolyl, 1,2,4-triazolyl, 1,2,3-triazolyl, 1,2,4-oxadiazolyl, 1,2,4-thiadiazolyl, 1,3,5-triazinyl, 1,2,4-triazinyl, 1,2,4,5-tetrazinyl and tetrazolyl.

The term “dendrimer” refers to a molecule containing a core and at least one dendron attached to the core. Each dendron is made up of at least one layer or generation of branched building units resulting in a branched structure with increasing number of branches with each generation of building units. The maximum number of dendrons attached to the core is limited by number of functional groups on the core. The core may have one or more functional groups suitable to bear a dendron and optionally an additional functional group for attachment of an agent suitable for targeting a specific organ or tissue, signalling or imaging.

The term “building unit” as used herein, refers to a branched molecule having at least three functional groups, one for attachment to the core or a previous generation of building units and at least two functional groups for attachment to the next generation of building units or forming the surface of the dendrimer molecule.

The term “generation” as used herein, refers to the number of layers of building units that make up a dendron or dendrimer. For example, a one generation dendrimer will have one layer of branched building units attached to the core, for example, Core-[[building unit]]u where u is the number of dendrons attached to the core. A two generation dendrimer has two layers of building units in each dendron attached to the core, for example, when the building unit has one branch point, the dendrimer may be: Core[[building unit][building unit]2]u, a three generation dendrimer has three layers of building units in each dendron attached to the core, for example Core-[[building unit][building unit]2[building unit]4]u, a 6 generation dendrimer has six layers of building units attached to the core, for example, Core-[[building unit][building unit]2[building unit]4[building unit]8[building unit]16[building unit]32]u, and the like. The last generation of building units (the outermost generation) provides the surface functionalisation of the dendrimer and the number of functional groups available for binding terminal groups. For example, in a dendrimer having a core with two dendrons attached (u=2), if each building unit has one branch point and there are 6 generations, the outermost generation has 64 building units and 128 functional groups available to bind terminal groups.

The term “sparingly soluble” as used herein, refers to a drug or pharmaceutically active agent that has a solubility between 1 mg/mL and 10 mg/mL in water. Drugs that have a solubility in water of less than 1 mg/mL are considered insoluble.

The term “pharmaceutically active agent” as used herein, refers to a compound that is used to exert a therapeutic effect in vivo. This term is used interchangeably with the term “drug”. The term “residue of a pharmaceutically active agent” refers to the portion of the macromolecule that is a pharmaceutically active agent when the pharmaceutically active agent has been modified by attachment to the macromolecule.

The term “oncology drug” as used herein, refers to a pharmaceutically active agent used to treat cancer, such as a chemotherapy drug.

As used herein, the term “solubilisation excipient” refers to a formulation additive that is used to solubilise insoluble or sparingly soluble drugs into an aqueous formulation. Examples include surfactants such as polyethoxylated caster oils including Cremophor EL, Cremophor RH 40 and Cremophor RH 60, D-α-tocopherol-polyethylene-glycol 1000 succinate, polysorbate 20, polysorbate 80, solutol HS 15, sorbitan monoleate, poloxamer 407, Labrasol and the like.

The macromolecules of the invention may be in the form of pharmaceutically acceptable salts. It will be appreciated however that non-pharmaceutically acceptable salts also fall within the scope of the invention since these may be useful as intermediates in the preparation of pharmaceutically acceptable salts or may be useful during storage or transport. Suitable pharmaceutically acceptable salts include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, maleic, citric, lactic, mucic, gluconic, benzoic, succinic, oxalic, phenylacetic, methanesulphonic, toluenesulphonic, benezenesulphonic, salicyclic sulphanilic, aspartic, glutamic, edetic, stearic, palmitic, oleic, lauric, pantothenic, tannic, ascorbic and valeric acids. Exemplary acid addition salts include, but are not limited to, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. Base salts include, but are not limited to, those formed with pharmaceutically acceptable cations, such as sodium, potassium, lithium, calcium, magnesium, ammonium and alkylammonium. Exemplary base addition salts include, but are not limited to, ammonium salts, alkali metal salts, for example those of potassium and sodium, alkaline earth metal salts, for example those of calcium and magnesium, and salts with organic bases, for example dicyclohexylamine, N-methyl-D-glucamine, morpholine, thiomorpholine, piperidine, pyrrolidine, a mono-, di- or tri-lower alkylamine, for example ethyl-, tert-butyl-, diethyl-, diisopropyl-, triethyl-, tributyl- or dimethyl-propylamine, or a mono-, di- or trihydroxy lower alkylamine, for example mono-, di- or triethanolamine. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion. It will also be appreciated that non-pharmaceutically acceptable salts also fall within the scope of the present disclosure since these may be useful as intermediates in the preparation of pharmaceutically acceptable salts or may be useful during storage or transport. Those skilled in the art of organic and/or medicinal chemistry will appreciate that many organic compounds can form complexes with solvents in which they are reacted or from which they are precipitated or crystallized. These complexes are known as “solvates”. For example, a complex with water is known as a “hydrate”. As used herein, the phrase “pharmaceutically acceptable solvate” or “solvate” refer to an association of one or more solvent molecules and a compound of the present disclosure. Examples of solvents that form pharmaceutically acceptable solvates include, but are not limited to, water, isopropanol, ethanol, methanol, DMSO, ethyl acetate, acetic acid, and ethanolamine.

Basic nitrogen-containing groups may be quarternized with such agents as lower alkyl halide, such as methyl, ethyl, propyl, and butyl chlorides, bromides and iodides; dialkyl sulfates like dimethyl and diethyl sulfate; and others.

### Macromolecules of the Invention

The macromolecules of the invention comprise:
i) a dendrimer comprising a core and at least one generation of building units, the outermost generation of building units having surface amino groups, wherein at least two different terminal groups are covalently attached to the surface amino groups of the dendrimer;
ii) a first terminal group which is a residue of a pharmaceutically active agent comprising a hydroxyl group;
iii) a second terminal group which is a pharmacokinetic modifying agent;
   wherein the first terminal group is covalently attached to the surface amino group of the dendrimer through a diacid linker, or a pharmaceutically acceptable salt thereof.

The dendrimers having surface amino groups have at least two different terminal groups covalently attached to the surface amino groups.

The first terminal group is a residue of a pharmaceutically active agent comprising a free hydroxyl group. The pharmaceutically active agent is attached to the surface amino group of the dendrimer through a diacid linker. The diacid linker forms an ester bond with the hydroxyl group of the pharmaceutically active agent and an amide bond with the surface amino group.

The pharmaceutically active agent may be any pharmaceutically active agent that has a hydroxyl group available for ester formation with the diacid linker and is administered to a subject to produce a therapeutic effect.

In some embodiments the pharmaceutically active agent is an oncology drug such as a taxane, a nucleoside or a kinase inhibitor, a steroid, an opioid analgesic, a respiratory drug, a central nervous system (CNS) drug, a hypercholesterolemic drug, an antihypertensive drug, an immunosuppressive drug, an antibiotic, a luteinising hormone releasing hormone (LHRH) agonist, a LHRH antagonist, an antiviral drug, an antiretroviral drug, an estrogen receptor modulator, a somatostatin mimic, an anti-inflammatory drug, a vitamin D2 analogue, a synthetic thyroxine, an antihistamine, an antifungal agent or a nonsteroidal anti-inflammatory drug (NSAID).

Suitable oncology drugs include taxanes such as paclitaxel, cabazitaxel and docetaxel, camptothecin and its analogues such as irinotecan and topotecan, other antimicrotubule agents such as vinflunine, nucleosides such as gemcitabine, cladribine, fludarabine capecitabine, decitabine, azacitidine, clofarabine and nelarabine, kinase inhibitors such as sprycel, temisirolimus, dasatinib, AZD6244, AZD1152, PI-103, R-roscovitine, olomoucine and purvalanol A, and epothilone B analogues such as Ixabepilone, anthracyclines such as amrubicin, doxorubicin, epirubicin and valrubicin, super oxide inducers such as trabectedin, proteosome inhibitors such as bortezomib and other topoisomerase inhibitors, intercalating agents and alkylating agents.

Suitable steroids include anabolic steroids such as testosterone, dihydrotestosterone and ethynylestradiol, and corticosteroids such as cortisone, prednisolone, budesonide, triamcinolone, fluticasone, mometasone, amcinonide, fluocinolone, fluocinonide, desonide, halcinonide, prednicarbate, fluocortolone, dexamethasone, betamethasone and fluprednidene.

Suitable opioid analgesics include morphine, oxymorphone, naloxone, codeine, oxycodone, methylnaltrexone, hydromorphone, buprenorphine and etorphine.

Suitable respiratory drugs include bronchodilators, inhaled steroids, and decongestants and more particularly salbutamol, ipratropium bromide, montelukast and formoterol.

Suitable CNS drugs include antipsychotic such as quetiapine and antidepressants such as venlafaxine.

Suitable drugs to control hypercholesterolemia include ezetimibe and statins such as simvastatin, lovastatin, atorvastatin, fluvastatin, pitavastatin, pravastatin and rosuvastatin.

Suitable antihypertensive drugs include losartan, olmesartan, medoxomil, metrolol, travoprost and bosentan.

Suitable immunosuppressive drugs include glucocorticoids, cytostatics, antibody fragments, anti-immunophilins, interferons, TNF binding proteins and more particularly, calcineurin inhibitors such as tacrolimus, mycophenolic acid and its derivatives such as mycophenolate mofetil, and cyclosporine.

Suitable antibacterial agents include antibiotics such as amoxicillin, meropenem and clavulanic acid.

Suitable LHRH agonists include goserelin acetate, deslorelin and

leuprorelin.

Suitable LHRH antagonists include cetrorelix, ganirelix, abarelix and degarelix.

Suitable antiviral agents include nucleoside analogs such as lamivudine, zidovudine, abacavir and entecavir and suitable antiretroviral drugs include protease inhibitors such as atazanavir, lopinavir and ritonavir.

Suitable selective estrogen receptor modulators include raloxifene and fulvestrant.

Suitable somastatin mimics include octreotide.

Suitable anti-inflammatory drugs include mesalazine and suitable NSAIDs include acetaminophen (paracetamol).

Suitable vitamin D2 analogues include paricalcitol.

Suitable synthetic thyroxines include levothyroxine.

Suitable anti-histamines include fexofenadine.

Suitable antifungal agents include azoles such as viriconazole.

In some embodiments the pharmaceutically active agent is sparingly soluble or insoluble in aqueous solution.

In particular embodiments the pharmaceutically active agent is selected from docetaxel, paclitaxel, testosterone, gemcitabine, camptothecin, irinotecan and topotecan, especially docetaxel, paclitaxel and testosterone.

In some embodiments the diacid linker comprises an alkyl chain interrupted by one or more oxygen, sulfur or nitrogen atoms.

The diacid linker that links the pharmaceutically active agent to the surface amino groups of the dendrimer have the formula:

—C(O)-J-C(O)—X—C(O)—
wherein X is selected from —C₁-C₁₀alkylene-, —(CH₂)ₛ-A-(CH₂)ₜ— and Q; —C(O)-J- is absent, an amino acid residue or a peptide of 2 to 10 amino acid residues, wherein the —C(O)— is derived from the carboxy terminal of the amino acid or peptide;
A is selected from —O—, —S—, —NR₁—, —N+ (R₁)₂—, —S—S—, —[OCH₂CH₂]ₜ—O—, —Y—, and —O—Y—O—;
Q is selected from Y or —Z═N—NH—S(O)_{w}—Y—;
Y is selected from cycloalkyl, heterocycloalkyl, aryl and heteroaryl;
Z is selected from —(CH₂)ₓ—C(CH₃)—, —(CH₂)ₓCH═, cycloalkyl and heterocycloalkyl;
R₁ is selected from hydrogen and C₁-C₄ alkyl;
s and t are independently selected from 1 and 2;
r is selected from 1, 2 and 3;
w is selected from 0, 1 and 2; and
x is selected from 1, 2, 3 and 4.

In some embodiments one or more of the following applies:
X is —C₁-C₆-alkylene, —CH₂-A-CH₂—, —CH₂CH₂-A-CH₂CH₂— or heteroaryl;
—C(O)-J is absent, an amino acid residue or a peptide of 2 to 6 amino acid residues,
wherein the —C(O)— is derived from the carboxy terminal of the amino acid or peptide;
A is selected from —O—, —S—, —S—S—, —NH—, —N(CH₃)—, —N⁺(CH₃)₂—, —O-1,2-phenyl-O—, —O-1,3-phenyl-O—, —O-1,4-phenyl-O—, —OCH₂CH₂O—, —[OCH₂CH₂]₂—O-and-[OCH₂CH₂]₃—O—;
Y is heteroaryl or aryl, especially thiophenyl, 3,4-propylenedioxythiophenyl or benzene;
Z is —(CH₂)ₓC(CH₃)—, —(CH₂)ₓCH═ and cycloalkyl, especially —CH₂CH₂C(CH₃)═, —CH₂CH₂CH₂C(CH₃)═, —CH₂CH₂CH₂CH═, cyclopentyl and cyclohexyl;
R₁ is hydrogen, methyl or ethyl, especially hydrogen or methyl, more especially methyl; one of s and t is 1 and the other is 1 or 2, especially were both s and t are 1;
r is 1 or 2, especially 2;
w is 1 or 2, especially 2; and
x is 2 or 3, especially 3.

In some embodiments, —C(O)-J- is absent. In other embodiments, —C(O)-J- is an amino acid residue or a peptide having 2 to 6 amino acid residues. In these embodiments, the N-terminus of the amino acid or peptide forms an amide bond with the —C(O)—X—C(O)— group. In some embodiments, the peptide is a peptide that comprises an amino acid sequence that is recognised and cleaved by an endogenous enzyme, such as a protease. In some embodiments, the enzyme is an intracellular enzyme. In other embodiments, the enzyme is an extracellular enzyme. In particular embodiments, the enzyme is one that is present in or around neoplastic tissue, such as tumor tissue. In some embodiments, the peptide is recognised by cathepsin B or a metalloprotease such as a neutral metalloproteinase (NMP), MMP-2 and MMP-9. Exemplary peptides include GGG, GFLG (SEQ ID NO: 1) and GILGVP (SEQ ID NO: 2).

In some embodiments, the diacid linker has the formula:

—C(O)—X—C(O)—

wherein X is —(CH₂)ₛ-A-(CH₂)ₜ—; A is —O—, —S— or —NR₁—; R₁ is selected from hydrogen and C₁-C₄ alkyl; and s and t are independently selected from 1 and 2. In some embodiments, X is —CH₂-A-CH₂—.

In particular embodiments the diacid linker is selected from: —C(O)—CH₂CH₂—C(O)—, —C(O)—CH₂CH₂CH₂—C(O)—, —C(O)—CH₂OCH₂—C(O)—, —C(O)—CH₂SCH₂—C(O)—, —C(O) CH₂NHCH₂—C(O)—, —C(O)—CH₂N(CH₃) CH₂—C(O)—, —C(O)—CH₂N⁺(CH₃)₂CH₂—C(O)—, —C(O)—CH₂—S—S—CH₂—C(O)—, —C(O)—OCH₂CH₂OCH₂CH₂OC(O)—,

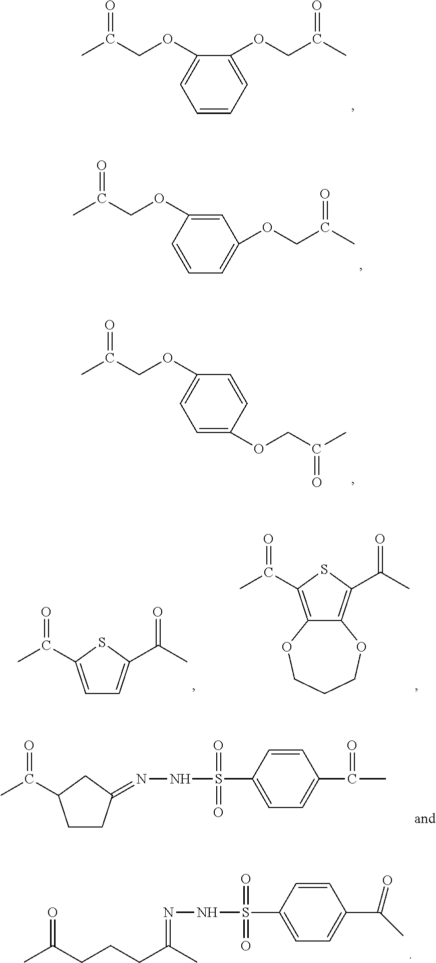

In some embodiments, the diacid linker is —C(O)—CH₂OCH₂—C(O)—.

In other embodiments, the diacid linker also comprises a peptide. Exemplary diacid linkers include:

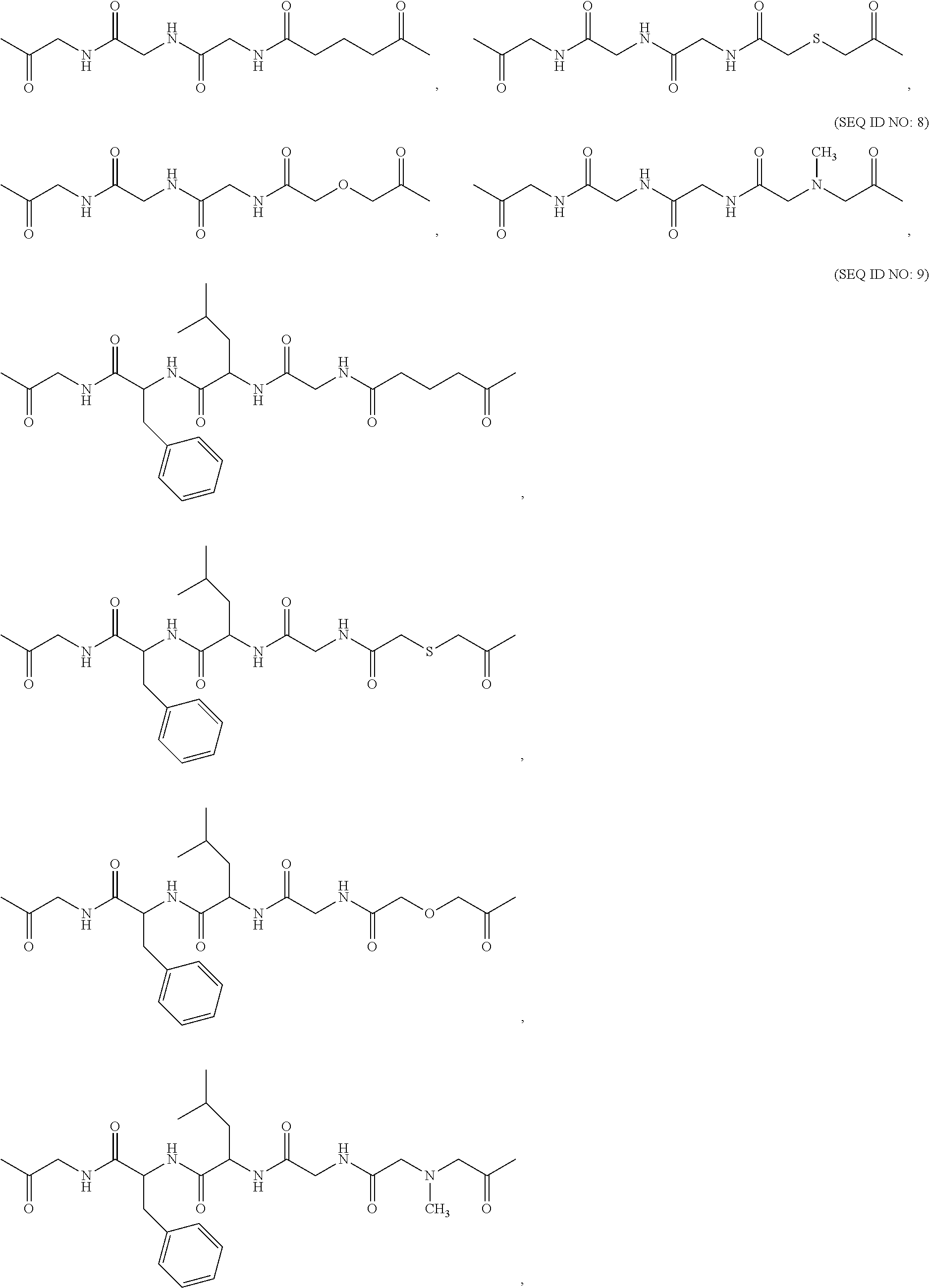

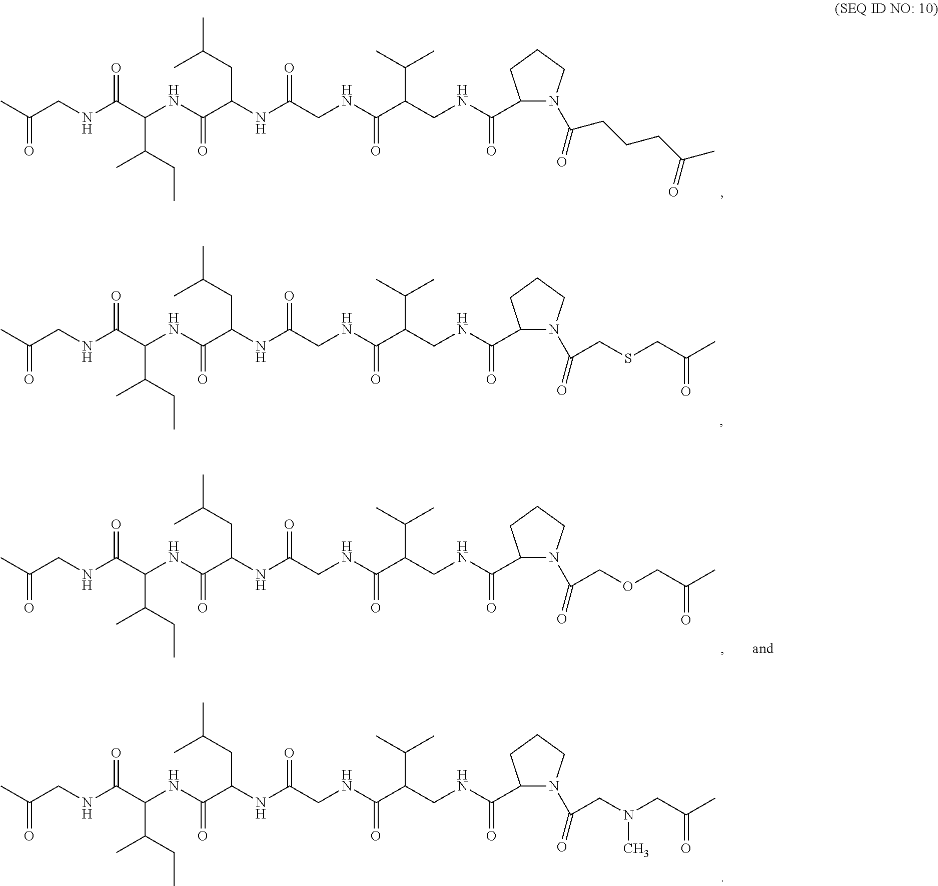

In some embodiments, the diacid linker is selected to provide a desired rate of release of the drug. For example, a rapid release may be required where the entire load of pharmaceutical agent is required in a short space of time whereas a slow release may be more suitable when a low constant therapeutic dose of pharmaceutically active agent is required.

In some embodiments, the rate of release is faster than the drug delivered independent of the macromolecule, especially at least twice as fast. In some embodiments, the drug is released more slowly than the drug independent of the macromolecule, especially where the drug is released at least two times slower, more especially the drug is released at least 10 times slower. In some embodiments, the drug is released at least 30 times slower as described in Example 39. Low rates of release may be particularly suitable where the macromolecule includes a targeting group, to enable release of the drug at the active site, but not in plasma. Low rates of release may also be suitable for drugs formulated to enable slow controlled release delivery over long periods of time, such as between 1 week and 6 months. The drug may be released from the macromolecule over a prolonged period of time, such as days, weeks or months. Fast release is preferably release greater than 50% within 0 to 480 minutes, especially within 0 to 120 minutes, and more especially within 5 to 60 minutes. Medium release preferably is release greater than 50% within 1 to 72 hours, especially within 2 to 48 hours. Slow release is preferably release of greater than 50% in greater than 2 days, especially 2 days to 6 months, and more especially within 5 days to 30 days.

The rate of release of the drug can be controlled by the selection of the diacid linker. Diacid linkers containing one or more oxygen atoms in their backbones, such as diglycolic acid, phenylenedioxydiacetic acid, and polyethylene glycol, or with a cationic nitrogen atom, tend to release drug at a rapid rate, diacid linkers having one sulfur atom in their backbone, such as thiodiacetic acid, have a medium rate of release and diacid linkers having one or more nitrogen atoms, two or more sulfur atoms, alkyl chains or heterocyclic or heteroaryl groups release the drug at a slow rate. The rate of release may be summarised by one or more —O—>—N⁺(R₁)₂—> one —S—> one —NR—>—N—NH—SO₂→—S—S→-alkyl->-heterocyclyl-.

It can be seen from Table 2, studies of macromolecules in plasma samples that the diglycolic acid (Experiment 3 (b)) released docetaxel at fast rate, with a half life of less than 22 hours, thiodiacetic acid linker (Experiment 8 (c)) released docetaxel at a medium rate, with a half life of a little more than 22 hours, extrapolated to around 24 to 30 hours and the glutaric acid linker (Experiment 5 (b)) released docetaxel at a slow rate with a half life of much greater than 22 hours, and predicted to be more than 2 days. Experiment 16 and 17 do not substantially release docetaxel in plasma but allow the macromolecule to be targeted to a tumor in which proteases can cleave the peptide sequence to provide the docetaxel at the site of action.

The rate of release may also be dependent on the identity of the pharmaceutically active agent.

In some embodiments, each pharmaceutically active agent is attached to the dendrimer with the same diacid linker. In other embodiments, two or more different diacid linkers are used allowing the pharmaceutically active agent to be released from the macromolecule at different rates.

In some embodiments, the macromolecule comprises a plurality of first terminal groups (T1) each comprising a cabazitaxel residue, wherein the cabazitaxel residue is covalently attached to a diglycolyl linker group, e.g.:

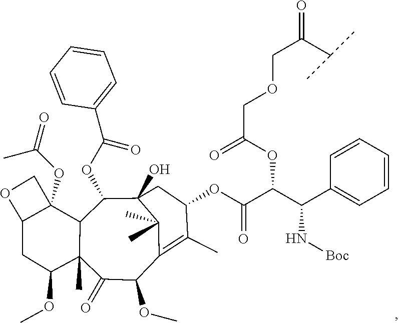

i.e. a cabazitaxel residue covalently attached to a diglycolyl linker via an ester linkage formed between an oxygen atom present as part of the cabazitaxel side-chain and a carbon atom of an acyl group present as part of the diglycolyl linker. The other acyl group of the diglycolyl linker forms an amide linkage with a nitrogen atom present in a surface amino group of the dendrimer.

In some embodiments, the macromolecule comprises a plurality of first terminal groups (T1) each comprising a cabazitaxel residue, wherein the cabazitaxel residue is covalently attached to a thiodiglycolyl/thiodiacetyl linker group, e.g.:

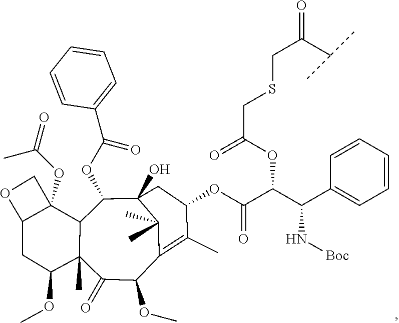

i.e. a cabazitaxel residue covalently attached to a thiodiacetyl/thiodiglycolyl linker via an ester linkage formed between an oxygen atom present as part of the cabazitaxel side-chain and a carbon atom of an acyl group present as part of the thiodiglycolyl/thiodiacetyl linker. The other acyl group of the thiodiacetyl linker forms an amide linkage with a nitrogen atom present in a surface amino group of the dendrimer.

In some embodiments, the macromolecule comprises a plurality of first terminal groups (T1) each comprising a cabazitaxel residue, wherein the cabazitaxel residue is covalently attached to a methyliminodiacetyl linker group, e.g.:

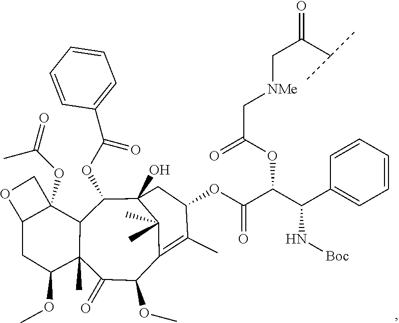

i.e. a cabazitaxel residue covalently attached to a methyliminodiacetyl linker via an ester linkage formed between an oxygen atom present as part of the cabazitaxel side-chain and a carbon atom of an acyl group present as part of the methyliminodiacetyl linker. The other acyl group of the methyliminodiacetyl linker forms an amide linkage with a nitrogen atom present in a surface amino group of the dendrimer.

In such embodiments, the cabazitaxel residue is:

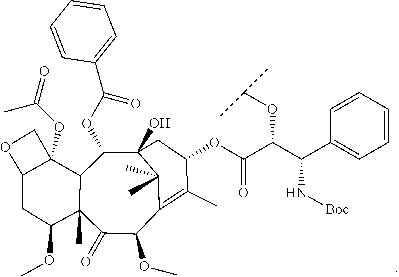

Upon in vivo administration, typically the dendrimer releases cabazitaxel, i.e.:

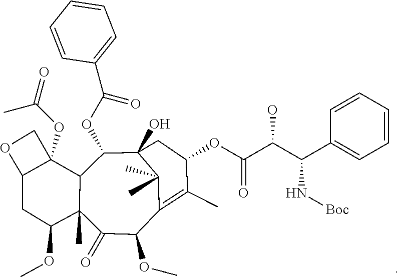

The second terminal group is a pharmacokinetic modifying agent, which may be any molecule or residue thereof that modifies or modulates the pharmacokinetic profile of the pharmaceutically active agent or the macromolecule including absorption, distribution, metabolism and/or excretion. In a particular embodiment, the pharmacokinetic modifying agent is an agent selected to prolong the plasma half-life of the pharmaceutically active agent, such that the macromolecule has a half life that is greater than the half-life of the native pharmaceutically active agent, or the marketed pharmaceutically active agent in a non-dendrimer formulation. Preferably the half life of the macromolecule or composition is at least 2 times and more preferably 10 times greater than the native pharmaceutically active agent, or the marketed pharmaceutically active agent in a non-dendrimer formulation.

In some embodiments, the second terminal group is polyethylene glycol (PEG), a polyalkyloxazoline such as polyethyloxazoline (PEOX), polyvinylpyrolidone and polypropylene glycol, especially PEG. In other embodiments, the second terminal group is a polyether dendrimer.

A PEG group is a polyethylene glycol group, i.e. a group comprising repeat units of the formula-CH₂CH₂O—. PEG materials used to produce the macromolecule according to some embodiments typically contain a mixture of PEGs having some variance in molecular weight (i.e., +10%), and therefore the molecular weight specified is typically an approximation of the average molecular weight of the PEG composition. For example, the term “PEG 2100” refers to polyethylene glycol having an average molecular weight of approximately 2100 Daltons, i.e. ± approximately 10% (i.e., PEG₁₉₀₀ to PEG₂₃₀₀). Three methods are commonly used to calculate MW averages: number average, weight average, and z-average molecular weights. As used herein, the phrase “molecular weight” is intended to refer to the weight-average molecular weight which can be measured using techniques well-known in the art including, but not limited to, NMR, mass spectrometry, matrix-assisted laser desorption ionization time of flight (MALDI-TOF), gel permeation chromatography or other liquid chromatography techniques, light scattering techniques, ultracentrifugation and viscometry.

In some embodiments, the PEG has a molecular weight of between 220 and 5500 Da. In some embodiments, the PEG has a molecular weight of 220 to 1100 Da, especially 570 and 1100 Da. In other embodiments, the PEG has a molecular weight of 1000 to 5500 Da, especially 1000 to 2500 Da or 1000 to 2300.

In some embodiments, the second terminal groups comprise PEG groups having an average molecular weight of at least 750 Daltons. In some embodiments, the second terminal groups comprise PEG groups having an average molecular weight in the range of from 1900 to 2300 Daltons. In some embodiments, the second terminal groups comprise PEG groups having an average molecular weight in the range of from 2000 to 2200 Daltons. In some embodiments, the second terminal groups comprise PEG groups having an average molecular weight of about 2100 Daltons.

In some embodiments, the PEG group has a polydispersity index (PDI) of between about 1.00 and about 1.50, between about 1.00 and about 1.25, or between about 1.00 and about 1.10. In some embodiments, the PEG group has a polydispersity index (PDI) of about 1.05. The term “polydispersity index” refers to a measure of the distribution of molecular mass in a given polymer sample. The polydispersity index (PDI) is equal to the weight average molecular weight (M_{w}) divided by the number average molecular weight (Mₙ) and indicates the distribution of individual molecular masses in a batch of polymers. The polydispersity index (PDI) has a value equal to or greater than one, but as the polymer approaches uniform change length and average molecular weight, the polydispersity index (PDI) will be closer to one.

In some embodiments, the PEG group is a methoxy-terminated PEG.

Where the second terminal group comprises a PEG group, the PEG group may be attached via any suitable means. In some embodiments, a PEG linking group is used to attach the PEG group. In some embodiments, the second terminal groups each comprise a PEG group covalently attached to a PEG linking group (L1) via an ether linkage formed between a carbon atom present in the PEG group and an oxygen atom present in the PEG linking group, and each second terminal group is covalently attached to a surface amino group via an amide linkage formed between a nitrogen atom present in a surface amino group and the carbon atom of an acyl group present in the PEG linking group. In some embodiments, the second terminal groups are each

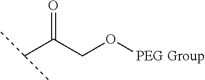

wherein the PEG group is a methoxy-terminated PEG having an average molecular weight in the range of from about 800 to 2500 Daltons, from about 800 to 1250 Daltons, or from about 1750 to 2500 Daltons.

In some embodiments, the macromolecules have controlled stoichiometry and/or topology. For example, the macromolecules are typically produced using synthetic processes that allow for a high degree of control over the number and arrangement of first and second terminal groups present. In some embodiments, each functionalised outer building unit contains one first terminal group and one second terminal group. In some embodiments, the dendrimer comprises surface units comprising an outer building unit attached to a first terminal group and a second terminal group, the surface units having the structure:

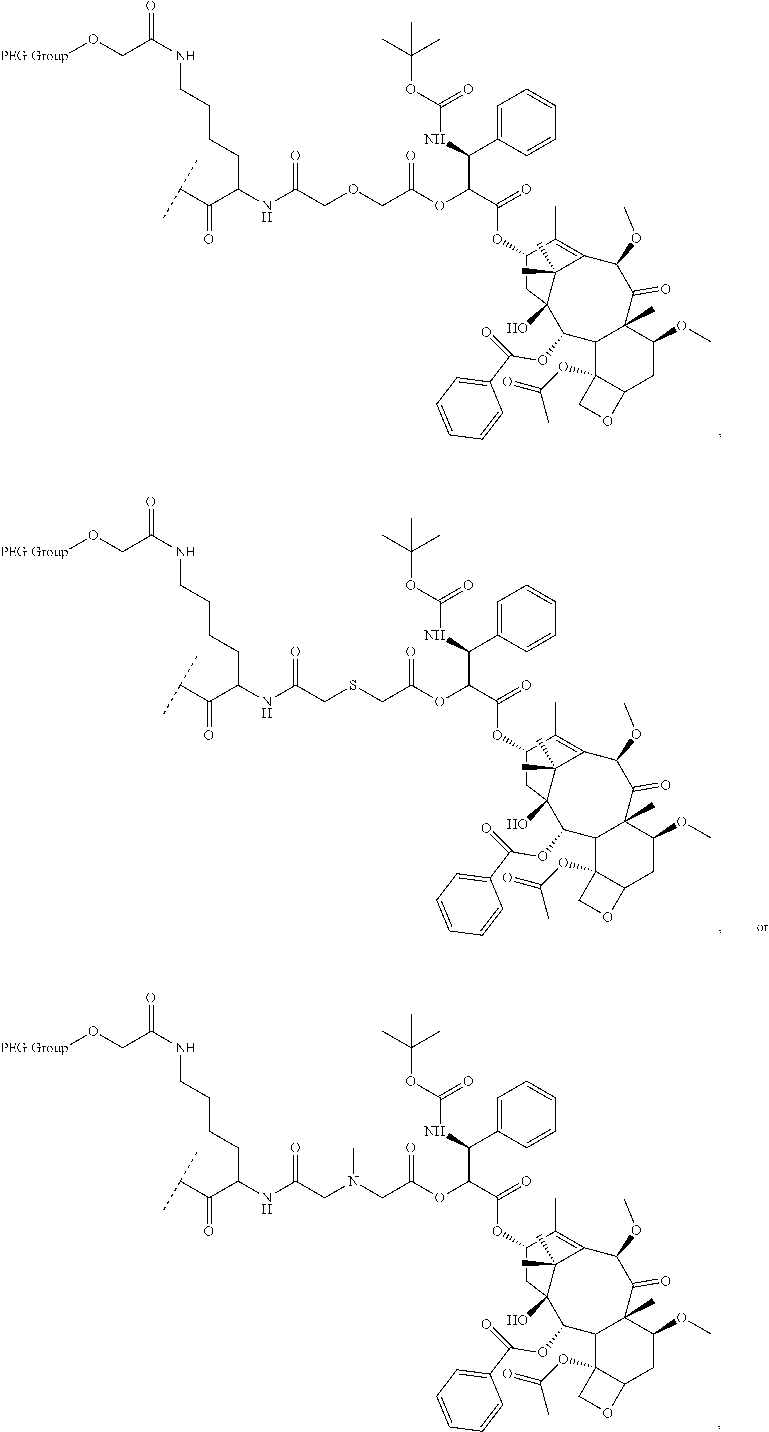

and wherein the PEG group is a methoxy-terminated PEG having an average molecular weight in the range of from about 800 to 2500 Daltons, from about 800 to 1250 Daltons, or from about 1750 to 2500 Daltons. In some embodiments, the dendrimer has from 28 to 32 surface units. In some embodiments, the dendrimer has from 30 to 32 surface units.

In some embodiments, the macromolecule comprises a third terminal group. The third terminal group is a blocking group that serves to block the reactivity of a surface amino group of the dendrimer. In particular embodiments, the blocking group is an acyl group such as a C₂-C₁₀ acyl group, especially acetyl. In other embodiments, the third terminal group is a second pharmaceutically active agent or a targeting agent.

In some embodiments where there is a first terminal group and a second terminal group, the ratio of first terminal group and second terminal group is between 1:2 and 2:1, especially 1:1.

In some embodiments where there is a first terminal group, a second terminal group and a third terminal group, the ratio is 1:1:1 to 1:2:2, especially 1:2:1.

In some embodiments, not all of the surface amino groups of the dendrimer are bound to a first terminal group, a second terminal group, or a third terminal group. In some embodiments, some of the surface amino groups remain free amino groups. In some embodiments at least 50% of the total terminal groups comprise one of a pharmacokinetic modifying agent or a pharmaceutically active agent, especially at least 75% or at least 80% of the terminal groups comprise one of a pharmacokinetic modifying agent or a pharmaceutically active agent. In particular embodiments, a pharmaceutically active agent is bound to greater than 14%, 25%, 27%, 30% 39%, 44% or 48% of the total number of surface amino groups. Where dendrimer is a G5 polylysine dendrimer, the total number of the pharmaceutically active agent is preferably greater than 15, and especially greater than 23 and more especially greater than 27. In some embodiments, the pharmacokinetic modifying agent is bound to greater than 15%, 25%, 30%, 33% or 46% of the total number of surface amino groups. Where dendrimer is a G5 polylysine dendrimer, the total number of pharmacokinetic modifying agents is preferably greater than 25, and especially greater than 30.

The macromolecule of the invention comprises a dendrimer in which the outermost generation of building units has surface amino groups. The identity of the dendrimer of the macromolecule is not particularly important, provided it has surface amino groups. For example, the dendrimer may be a polylysine, polylysine analogue, polyamidoamine (PAMAM), polyethyleneimine (PEI) dendrimer or polyether hydroxylamine (PEHAM) dendrimer.

The dendrimer comprises a core and one or more dendrons made of one or more building units. The building units are built up in layers referred to as generations.

In some embodiments, the building unit is a polyamine, more preferably a di or tri-amino with a single carboxylic acid. Preferably the molecular weight of the building unit is from 110 Da to 1 KDa. In some embodiments, the building unit is lysine or lysine analogue selected from:
Lysine 1: having the structure:

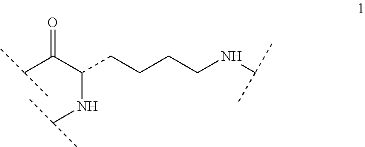

Glycyl-Lysine 2 having the structure:

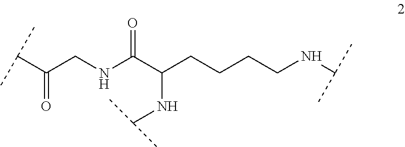

Analogue 3, having the structure below, where a is an integer of 1 or 2; b and c are the same or different and are integers of 1 to 4:

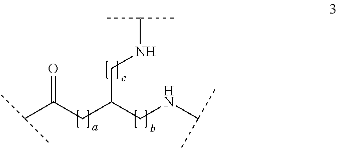

Analogue 4, having the structure below, where a is an integer of 0 to 2; b and c are the same or different and are integers of 2 to 6:

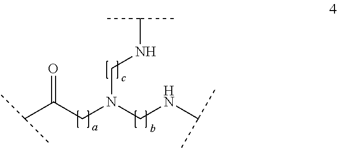

Analogue 5, having the structure below, where a is an integer of 0 to 5; b and c are the same or different and are integers of 1 to 5:

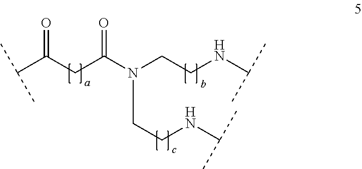

Analogue 6, having the structure below, where a is an integer of 0 to 5; b and c are the same or different and are integers of 0 to 5:

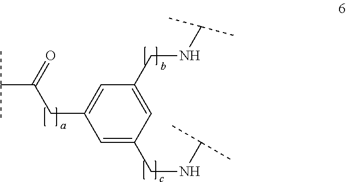

Analogue 7, having the structure below, where a is an integer of 0 to 5; b and c are the same or different and are integers of 1 to 5:

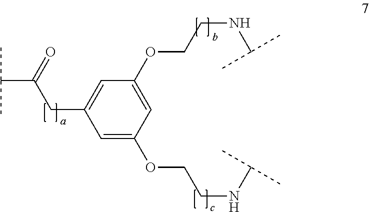

Analogue 8, having the structure below, where a is an integer of 0 to 5; b, c and d are the same or different and are integers of 1 to 5:

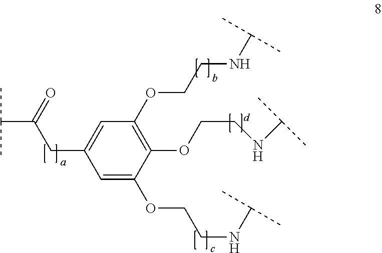

Analogue 9, having the structure below, where a is an integer of 0 to 5; b and c are the same or different and are integers of 1 to 5:

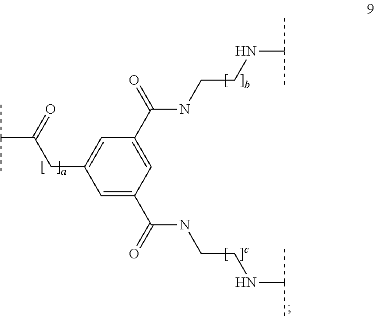

and furthermore, the alkyl chain moieties (eg: —C—C—C—) of the building units may be understood to include alkoxy fragments such as C—O—C or C—C—O—C—C where one or more non-adjacent carbon atom is replaced with an oxygen atom, provided that such a substitution does not form a O—C—X group where X is O or N.

In some embodiments the building unit is an amidoamine building unit with the structure 10:

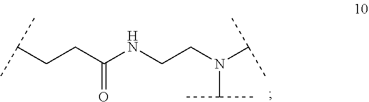

an etherhydroxyamine building unit with the structure 11:

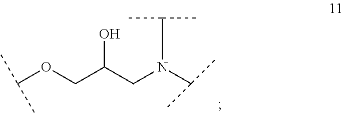

or a propyleneimine building unit with the structure 12:

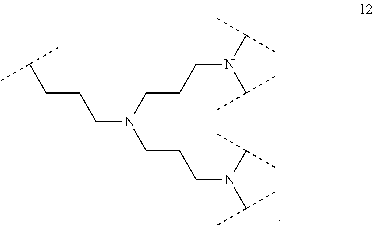

In a preferred embodiment, the building units are selected from Lysine 1, Glycyl-Lysine 2 or Lysine analogue 5:

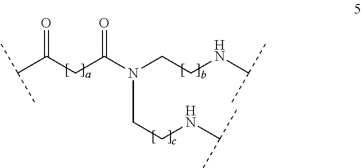

where a is an integer of 0 to 2 or the alkyl link is C—O—C; b and c are the same or different and are integers of 1 to 2; especially where the building units are lysine.

In some embodiments, the core is a monoamine compound, diamine compound, triamine compound, tetraamine compound or pentaamine compound, one or more of the amine groups having a dendron comprising building units attached thereto. In particular embodiments, the molecular weight of the building unit is from 110 Da to 1 KDa.

Suitable cores include benzhydrylamine (BHA), a benzhydrylamide of lysine (BHALys) or a lysine analogue, or:

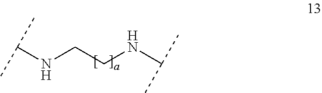

where a is an integer of 1 to 9, preferably 1 to 5;

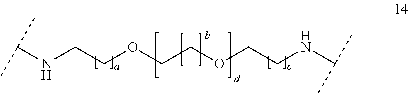

where a, b and c, which may be the same or different, and are integers of 1-5, and d is an integer from 0-100, preferably 1-30;

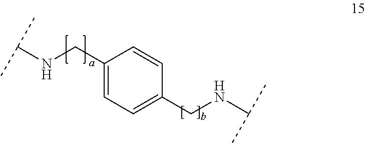

where a and b, may be the same or different, and are integers of 0 to 5;

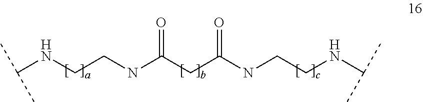

where a and c, which may be the same or different, are integers of 1 to 6 and where c is an integer from 0 to 6;

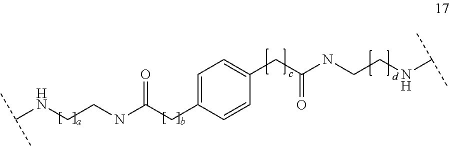

where a and d, which may be the same or different, are integers of 1 to 6 and where b and c, which may be the same or different, are integers from 0 to 6;

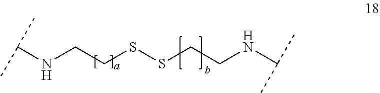

where a and b are the same or different and are integers of 1 to 5, especially 1 to 3, more especially 1;
a triamine compound selected from:

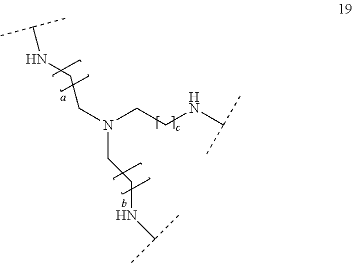

where a, b and c, which may be the same or different, are integers of 1 to 6;

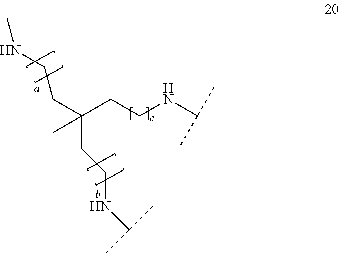

where a, b and c, which may be the same or different, are integers of 0 to 6;

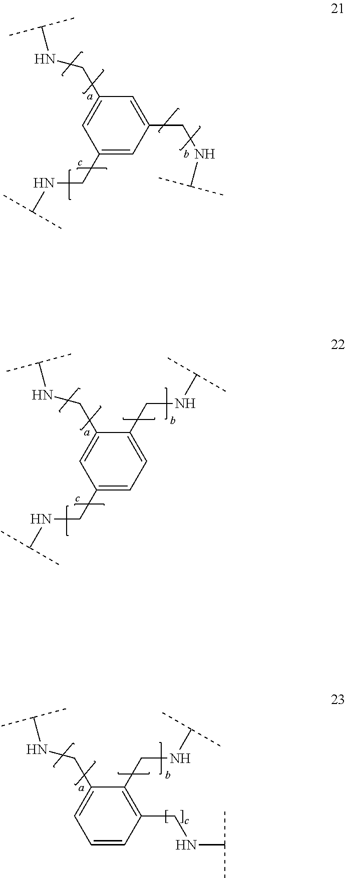

where a, b and c, which may be the same or different, are integers of 0 to 6;

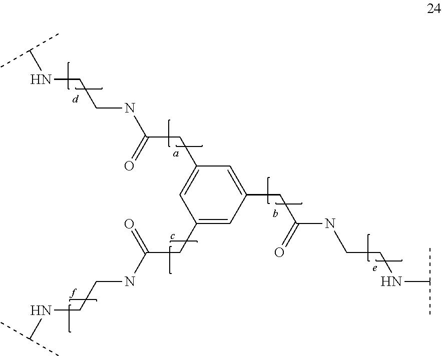

where a, b and c, which may be the same or different, are integers of 0 to 6; and d, e and f, which may be the same or different, are integers of 1 to 6;

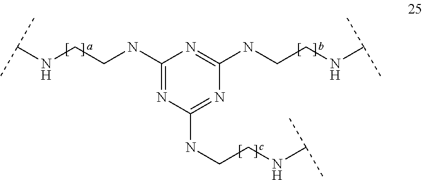

where a, b and c, which may be the same or different, are integers of 1 to 6;

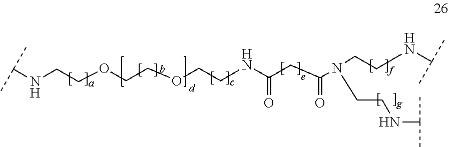

wherein a, b and c, which may be the same or different, are integers of 1 to 5, d is an integer from 1 to 100, preferably 1 to 30, e is an integer from 0 to 5 and f and g are the same or different and are integers from 1 to 5;
or a tetraamine compound selected from

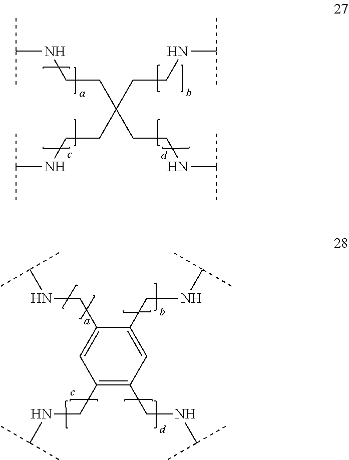

where a, b, c and d, which may be the same or different, are integers of 0 to 6;

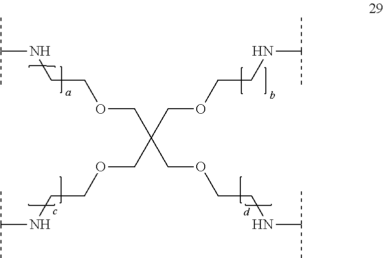

where a, b, c and d, which may be the same or different, are integers of 1 to 6;

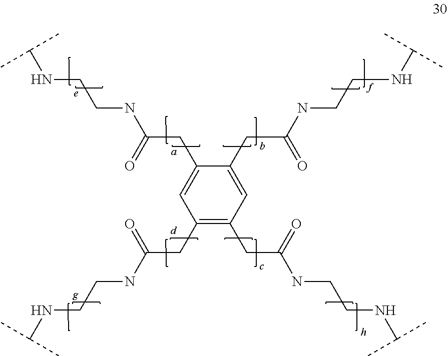

where a, b, c and d, which may be the same or different, are integers of 0 to 6; and e, f, g and h, which may be the same or different, are integers of 1 to 6; and furthermore, the alkyl chain moieties (eg: —C—C—C—) of the building units may be understood to include alkoxy fragments such as C—O—C or C—C—O—C—C where one or more non-adjacent carbon atom is replaced with an oxygen atom, provided that such a substitution does not form a O—C—X group where X is O or N.

In some embodiments, the core has at least two amino functional groups, one of which has attached a targeting moiety either directly or through a spacer group. At least one of the remaining functional groups of the core having a dendron attached as described in WO 2008/017125.

In some embodiments, the core unit (C) of the dendrimer is covalently attached to two building units via amide linkages, each amide linkage being formed between a nitrogen atom present in the core unit and the carbon atom of an acyl group present in a building unit. Accordingly, the core unit may for example be formed from a core unit precursor comprising two amino groups. Any suitable diamino-containing molecule may be used as the core unit precursor. In some embodiments, the core unit is:

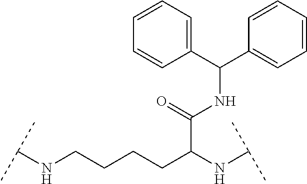

and may, for example, be formed from a core unit precursor:

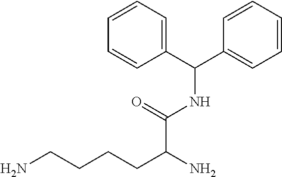 [SMILES: NCCCCC(N)C(=O)NC(c1ccccc1)c1ccccc1]

having two reactive (amino) nitrogens.

The targeting agent is an agent that binds to a biological target cell, organ or tissue with some selectivity thereby assisting in directing the macromolecule to a particular target in the body and allowing its accumulation at that target cell, organ or tissue. The targeting group may in addition provide a mechanism for the macromolecule to be actively taken into the cell or tissue by receptor mediated endocytosis.

Particular examples include lectins and antibodies and other ligands (including small molecules) for cell surface receptors. The interaction may occur through any type of bonding or association including covalent, ionic and hydrogen bonding, Van der Waals forces.

Suitable targeting groups include those that bind to cell surface receptors, for example, the folate receptor, adrenergic receptor, growth hormone receptor, luteinizing hormone receptor, estrogen receptor, epidermal growth factor receptor, fibroblast growth factor receptor (eg FGFR2), IL-2 receptor, CFTR and vascular epithelial growth factor (VEGF) receptor.

In some embodiments, the targeting agent is luteinising hormone releasing hormone (LHRH) or a derivative thereof that binds to luteinising hormone releasing hormone receptor. LHRH has the sequence: pyroGlu-His-Trp-Ser-Tyr-Gly-Leu-Arg-Pro-Gly-NH₂ (SEQ ID NO: 3). Suitable derivatives of LHRH include those in which one of residues 4-7 are replaced by another amino acid, especially residue 6 (Gly). In some embodiments, the replacement amino acid residue is suitably one that has a side chain capable of forming a bond with the core or with the spacer. In some embodiments the derivative is LHRH Gly6Lys, LHRH Gly6Asp or LHRH Gly6Glu, especially LHRH Gly6Lys. In other embodiments, the derivative is LHRH Gly6Trp (deslorelin). This receptor is often found or overexpressed in cancer cells, especially in breast, prostate, ovarian or endometrial cancers.

In some embodiments, the targeting agent is LYP-1, a peptide that targets the lymphatic system of tumors but not the lymphatic system of normal tissue. LYP-1 is a peptide having the sequence H-Cys-Gly-Asn-Lys-Arg-Thr-Arg-Gly-Cys-OH (SEQ ID NO: 4) and in which the peptide is in cyclic form due to a disulfide bond between the sulphur atoms of the two cysteine residues.

In some embodiments, the targeting agent may be an RGD peptide. RGD peptides are peptides containing the sequence-Arg-Gly-Asp-. This sequence is the primary integrin recognition site in extracellular matrix proteins.

Antibodies and antibody fragments such as scFvs and diabodies known to interact with receptors or cellular factors include CD20, CD52, MUC1, Tenascin, CD44, TNF-R, especially CD30, HER2, VEGF, EGF, EFGR and TNF-α.

In some embodiments the targeting agent may be folate. Folate is a vitamin that is essential for the biosynthesis of nucleotide bases and is therefore required in high amounts in proliferating cells. In cancer cells, this increased requirement for folic acid is frequently reflected in an overexpression of the folate receptor which is responsible for the transport of folate across the cell membrane. In contrast, the uptake of folate into normal cells is facilitated by the reduced folate carrier, rather than the folate receptor. The folate receptor is upregulated in many human cancers, including malignancies of the ovary, brain, kidney, breast, myeloid cells and the lung and the density of folate receptors on the cell surface appears to increase as the cancer develops.

Estrogens may also be used to target cells expressing estrogen receptor.

The targeting agent may be bound to the dendrimer core directly or preferably through a spacer. The spacer group may be any divalent group capable of binding to both the functional group of the core and the functional group on the targeting agent. The size of the spacer group is preferably sufficient to prevent any steric crowding. Examples of suitable spacer groups include alkylene chains and alkylene chains in which one or more carbon atoms is replaced by a heteroatom selected from —O—, —S—, or NH. The alkylene chain terminates with functional groups suitable for attachment to both the core functional group and the targeting agent. Exemplary spacer groups include X—(CH₂)ₚ—Y, X—(CH₂O)ₚ—CH₂—Y, X—(CH₂CH₂O)ₚ—CH₂CH₂—Y and X—(CH₂CH₂CH₂O)ₚCH₂CH₂CH₂—Y, where X and Y are functional groups for binding with or bound to the core and the targeting agent respectively, and p is an integer from 1 to 100, especially 1 to 50 or 1 to 25.

In some embodiments, the targeting group may be bound to the surface amino groups as third functional group. In some embodiments, 1 to 32 targeting groups are bound to the surface, especially, 1 to 10 are bound, more especially 1 to 4 are bound.

In some embodiments, the targeting agent and the spacer group are modified to facilitate reaction. For example, the spacer group may include an azide functional group and the targeting agent may include an alkyne group or the spacer group is modified with an alkyne and the targeting agent modified with an azide and the two groups are conjugated using a click reaction.

In some embodiments the functional group of the core that does not bear a dendron may be bound to biotin, optionally through a spacer group described above, and the macromolecule reacted with an avidin-antibody or avidin-biotin-antibody complex. Each avidin complex may bind up to 4 macromolecule-biotin conjugates or a combination of macromolecule-biotin conjugates and antibody-biotin conjugates.

In particular embodiments, the core is BHA or BHALys or NEOEOEN[SuN(PN)₂].

In some embodiments, the dendrimer has 1 to 5 dendrons attached to the core, especially 2 to 4 dendrons, more especially 2 or 3 dendrons.

In some embodiments, the dendrimer has 1 to 8 generations of building units, especially 2 to 7 generations, 3 to 6 generations, more especially 4 to 6 generations.

It will be appreciated that the dendrons of the dendrimer may for example be synthesised to the required number of generations through the attachment of building units (BU) accordingly. In some embodiments each generation of building units (BU) may be formed of the same building unit, for example all of the generations of building units may be lysine building units. In some other embodiments, one or more generations of building units may be formed of different building units to other generations of building units.

In some embodiments the dendrimer is a five generation building unit dendrimer. A five generation building unit dendrimer is a dendrimer having a structure which includes five building units which are covalently linked to another, for example in the case where the building units are lysines, it may comprise the substructure:

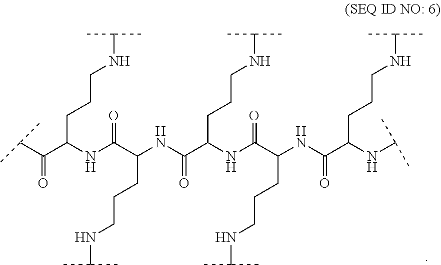

In some embodiment, the dendrimer has complete generations of building units. For example, in the cases of a five generation building unit dendrimer, in some embodiments the dendrimer has five complete generations of building units. With a core having two reactive amine groups, such a dendrimer will comprise 62 building units (i.e. core unit+2 BU+4 BU+8 BU+16 BU+32 BU). However, it will be appreciated that, due to the nature of the synthetic process for producing the dendrimers, one or more reactions carried out to produce the dendrimers may not go fully to completion. Accordingly, in some embodiments, the dendrimer may comprise an incomplete generations of building units. For example, a population of dendrimers may be obtained, in which the dendrimers have a distribution of numbers of building units per dendrimer. In some embodiments, a population of dendrimers is obtained which has a mean number of building units per dendrimer of at least 55, or at least 56, or at least 57, or at least 58, or at least 59, or at least 60. In some embodiments, a population of dendrimers is obtained in which at least 60%, at least 70%, at least 80%, at least 90% or at least 95% of the dendrimers have 55 or more building units. In some embodiments, a population of dendrimers is obtained in which at least 60%, at least 70%, at least 80%, at least 90% or at least 95% of the dendrimers have 60 or more building units.

In some embodiments, no more than one quarter of the nitrogen atoms present in the outer generation of building units are unsubstituted. In some embodiments, no more than one fifth of the nitrogen atoms present in said outer generation of building units are unsubstituted. In some embodiments, no more than one sixth of the nitrogen atoms present in said outer generation of building units are unsubstituted. In some embodiments, no more than one eighth of the nitrogen atoms present in said outer generation of building units are unsubstituted. In some embodiments, no more than one tenth of the nitrogen atoms present in said outer generation of building units are unsubstituted.

In some embodiments, no more than 20 nitrogen atoms present in the outer generation of building units are unsubstituted. In some embodiments, no more than 10 nitrogen atoms present in the outer generation of building units are unsubstituted. In some embodiments, no more than 5 nitrogen atoms present in the outer generation of building units are unsubstituted. In some embodiments, no more than 3 nitrogen atoms present in the outer generation of building units are unsubstituted. In some embodiments, no more than 2 nitrogen atoms present in the outer generation of building units are unsubstituted. In some embodiments, no more than 1 nitrogen atom present in the outer generation of building units are unsubstituted. In some embodiments, substantially all of the nitrogen atoms present in the outer generation of building units are substituted.

In some embodiments, the macromolecule comprises:
a core (C); and
building units (BU), each building unit being a lysine residue or an analogue thereof;
wherein the core unit is covalently attached to two building units via amide linkages, each amide linkage being formed between a nitrogen atom present in the core unit and the carbon atom of an acyl group present in a building unit;
the macromolecule being a five generation building unit macromolecule;
wherein building units of different generations are covalently attached to one another via amide linkages formed between a nitrogen atom present in one building unit and the carbon atom of an acyl group present in another building unit;
the macromolecule further comprising:
a plurality of first terminal groups (T1) each comprising a cabazitazel residue, wherein the cabazitaxel residues are covalently attached to a diglycolyl, thiodiacetyl or methyliminodiacetyl linker group; and
a plurality of second terminal groups (T2) each comprising a PEG group;
wherein at least one third of the nitrogen atoms present in outer building units are each covalently attached to a first terminal group; and
at least one third of the nitrogen atoms present in outer building units are each covalently attached to a second terminal group;
or a pharmaceutically acceptable salt thereof.

For such macromolecules, in some embodiments one or more of the following applies:
the core (C) is:

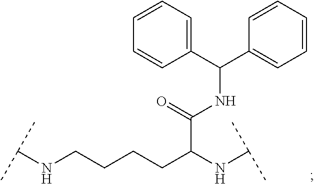

the building units (BU) are each

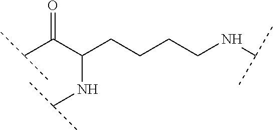

or, more preferably,

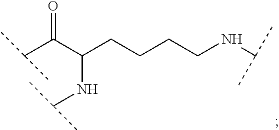

wherein the acyl group of each building unit provides a covalent attachment point for attachment to the core or to a previous generation building unit; and wherein each nitrogen atom provides a covalent attachment point for covalent attachment to a subsequent generation building unit, a first terminal group or a second terminal group; the dendrimer has five complete generations of building units;
each first terminal group (T1) comprises a cabazitazel residue, wherein the cabazitaxel residues are covalently attached to a diglycolyl, thiodiglycolyl/thiodiacetyl or methyliminodiacetyl linker group, i.e.:

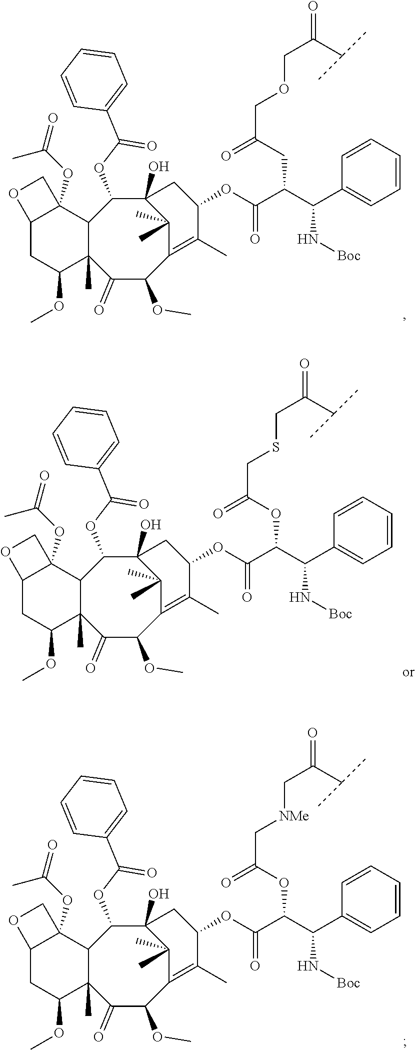

the second terminal groups comprise PEG groups having a mean molecular weight of at least 750 Daltons; or comprise PEG groups having an average molecular weight in the range of from 800 to 2500 Daltons; or comprise PEG groups having an average molecular weight in the range of from 800 to 1250 Daltons; or comprises PEG groups having an average molecular weight in the range of from 1750 to 2500 Daltons; or comprise PEG groups having a average molecular weight in the range of from 1900 to 2300 Daltons; the second terminal groups comprise methoxy-terminated PEG groups;
the second terminal groups each comprise a PEG group covalently attached to a PEG linking group (L1) via an ether linkage formed between a carbon atom present in the PEG group and an oxygen atom present in the PEG linking group, and each second terminal group is covalently attached to a building unit via an amide linkage formed between a nitrogen atom present in a building unit and the carbon atom of an acyl group present in the PEG linking group; or the second terminal groups are each

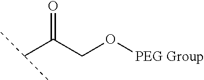

wherein the PEG group is a methoxy-terminated PEG having an average molecular weight in the range of from about 800 to 2500 Daltons, or from about 800 to 1250 Daltons, or from about 1750 to 2500 Daltons;
the dendrimer comprises surface units comprising an outer building unit attached to a first terminal group and a second terminal group, the surface units having the structure:

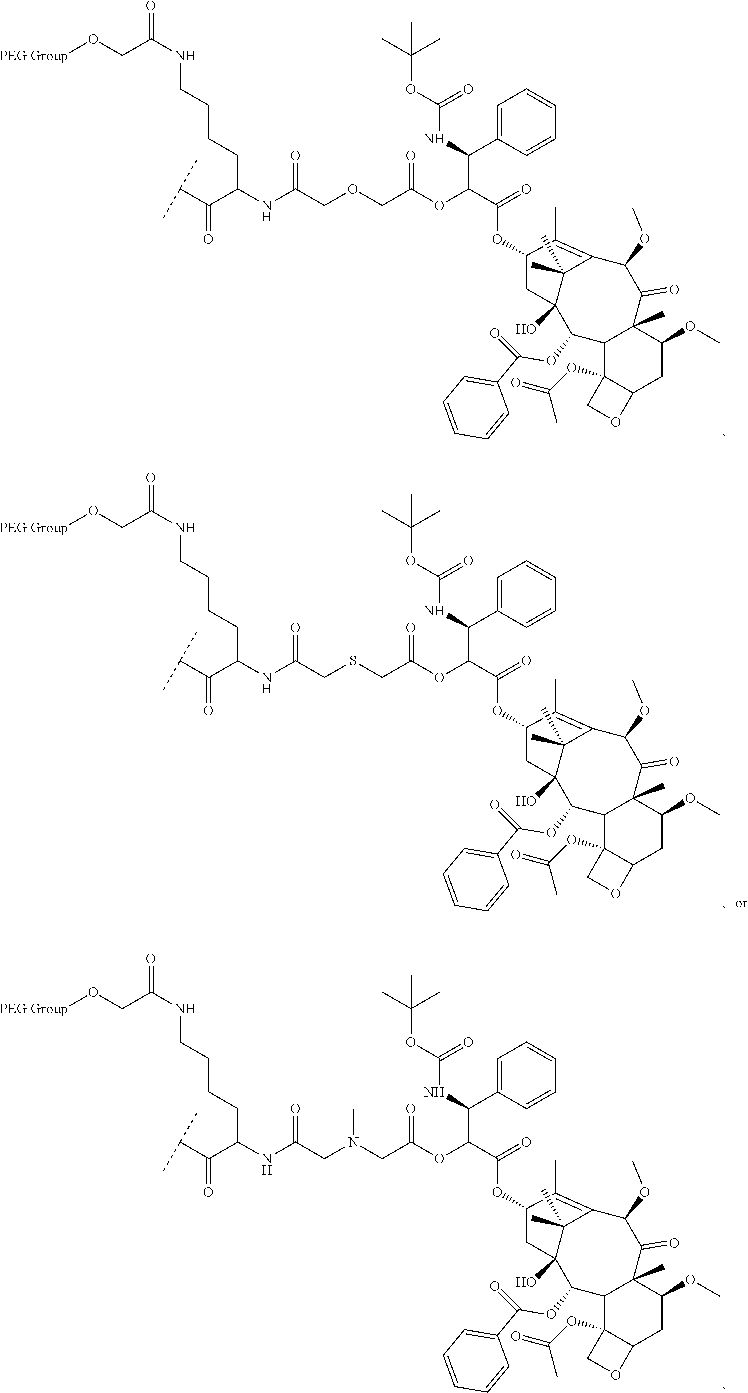

and wherein the PEG group is a methoxy-terminated PEG having an average molecular weight in the range of from about 800 to 2500 Daltons, or from about 800 to 1250 Daltons, or from about 1750 to 2500 Daltons, or from about 1900 to 2300 Daltons; the dendrimer has from 28 to 32 surface units, preferably from 30 to 32 surface units; at least 40% of the nitrogen atoms present in the outer building units are each covalently attached to a first terminal group; and at least 40% of the nitrogen atoms present in the outer building units are each covalently attached to a second terminal group;
the five generations of building units are complete generations, and wherein the outer generation of building units provides 64 nitrogen atoms for covalent attachment to a first terminal group or a second terminal, wherein from 26 to 32 first terminal groups are covalently attached to one of said nitrogen atoms, and wherein from 28 to 32 second terminal groups are each covalently attached to one of said nitrogen atoms;
from 28 to 32 first terminal groups are each covalently attached to one of said nitrogen atoms;
from 29 to 31 first terminal groups are each covalently attached to one of said nitrogen atoms;
no more than one fifth of the nitrogen atoms present in said outer generation of building units are unsubstituted; and
no more than 10 nitrogen atoms present in said outer generation of building units are unsubstituted.

In some embodiments, the macromolecule is:

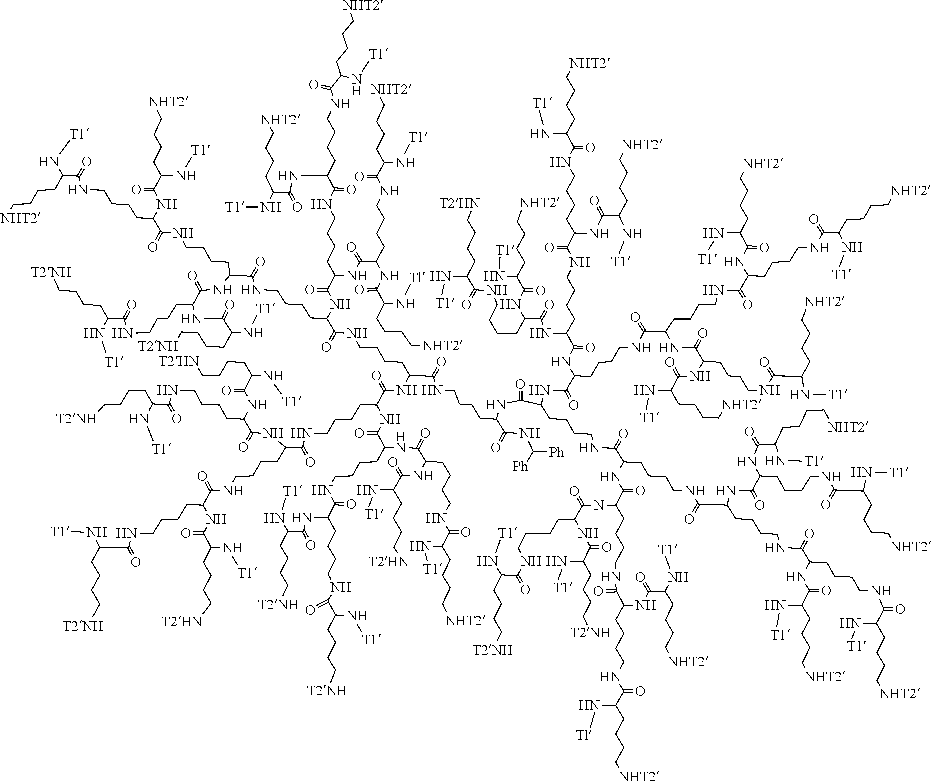

in which T1′ represents

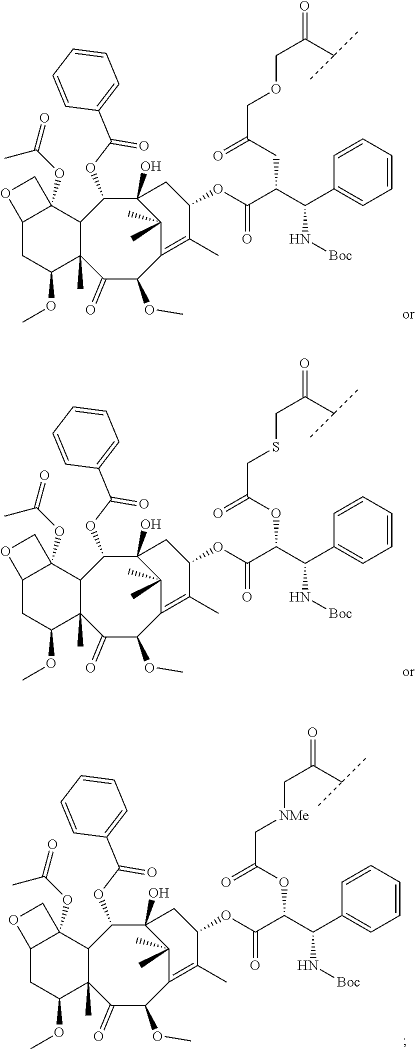

or T1′ represents H, wherein less than 5 of T1′ are H; and
T2′ represents

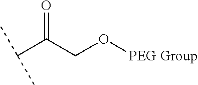

wherein the PEG group is a methoxy-terminated PEG having an average molecular weight in the range of from about 800 to 2500 Daltons, or from about 800 to 1250 Daltons, or from about 1750 to 2500 Daltons, or form about 1900 to 2300 Daltons, or T2′ represents H, and wherein less than 5 of T2′ are H.

In some embodiments, the macromolecule has a molecular weight in the range of from 50 to 300 kDa. In some embodiments, the macromolecule has a molecular weight in the range of from 75 to 200 kDa. In one example, the macromolecule has a molecular weight in the range of from 90 to 150 kDa.

In some embodiments, where the pharmaceutically active agent is cabazitaxel, the in vitro half-life for cabazitaxel release from the macromolecule in PBS (phosphate-buffer saline) at pH 7.4 and at 37° C. is in the range of from 20 to 100 hours. In some embodiments, the in vitro half-life for cabazitaxel release from the macromolecule in PBS at pH 7.4 and at 37° C. is in the range of from 24 to 60 hours. In some embodiments, the in vitro half-life for cabazitaxel release from the macromolecule in PBS at pH 7.4 and at 37° C. is in the range of from 30 to 60 hours. In some embodiments, the in vitro half-life for cabazitaxel release from the macromolecule in PBS at pH 7.4 and at 37° C. is in the range of from 30 to 50 hours.

The macromolecule of the invention may be nanoparticulate having a particulate diameter of below 1000 nm, for example, between 5 and 1000 nm, especially 5 and 500 nm, more especially 5 to 400 nm, such as 5 to 50 nm, especially between 5 and 20 nm. In particular embodiments, the composition contains macromolecules with a mean size of between 5 and 20 nm. In some embodiments, the macromolecule has a molecular weight of at least 30 kDa, for example, 40 to 150 kDa or 40 to 300 kDa.

In some embodiments, the macromolecules of the invention have a particle size that is suitable for taking advantage of the Enhanced Permeability and Retention Effect (EPR effect) in tumors and inflammatory tissue. Blood vessels formed in tumors are formed quickly and are abnormal because of poorly-aligned defective endothelial cells, a lack of smooth muscle layer and/or innervation with a wider lumen. This makes the tumor vessels permeable to particles of a size that would not normally exit the vasculature and allow the macromolecules to collect in tumor tissue. Furthermore, tumor tissues lack effective lymphatic drainage therefore once the macromolecules have entered the tumor tissue, they are retained there. Similar accumulation and retention is found in sites of inflammation.

The macromolecule of the invention may have a loading of pharmaceutically active agent of 2, 4, 8, 16, 32, 64 or 120 residues, especially 16, 32 or 64 residues per macromolecule.

Methods of making dendrimers are known in the art. For example, the dendrimers of the macromolecule may be made by a divergent method or a convergent method or a mixture thereof.

In the divergent method each generation of building units is sequentially added to the core or an earlier generation. The surface generation having one or both of the surface amino groups protected. If one of the amino groups is protected, the free amino group is reacted with one of the linker, the linker-pharmaceutically active agent or the pharmacokinetic modifying agent. If both amino groups are protected, they are protected with different protecting groups, one of which may be removed without removal of the other. One of the amino protecting groups is removed and reacted with one of the linker, the linker-pharmaceutically active agent or the pharmacokinetic modifying agent. Once the initial terminal group has been attached to the dendrimer, the other amino protecting group is removed and the other of the first and second terminal group is added. These groups are attached to the surface amino groups by amide formation as known in the art.

In the convergent method, each generation of building units is built up on the previous generation to form a dendron. The first and second terminal groups may be attached to the surface amino groups as described above before or after attachment of the dendron to the core.

In a mixed approach, each generation of building units is added to the core or a previous generation of building units. However, before the last generation is added to the dendrimer, the surface amino groups are functionalised with terminal groups, for example, a first and second terminal group, a first and third terminal group or a second and third terminal group. The functionalised final generation is then added to the subsurface layer of building units and the dendron is attached to the core.

The pharmaceutically active agent is reacted with one of the carboxylic acids of the linker by ester formation as known in the art. For example, an activated carboxylic acid is formed, such as an acid chloride or an anhydride is used and reacted with the hydroxy group of the pharmaceutically active agent. If the pharmaceutically active agent has more than one hydroxy group, further hydroxy groups may be protected.

In the case where a targeting agent is attached to the core, a functional group on the core may be protected during formation of the dendrimer then deprotected and reacted with the targeting agent, the spacer group or the targeting agent-spacer group. Alternatively, the core may be reacted with the spacer group or targeting agent-spacer group before the formation of the dendrimer.

Suitable protecting groups, methods for their introduction and removal are described in Greene & Wuts, Protecting Groups in Organic Synthesis, Third Edition, 1999.

In the case of macromolecules which comprise cabazitaxel covalently attached to a group of formula-C(O)CH₂ACH₂C(O)— where A is —O—, —S— or —NR₁— (e.g. a diglycolyl, thiodiacetyl, or methyliminodiacetyl linker group), second terminal groups which comprise a PEG group, and five generations of building units which are lysine residues or an analogue thereof, the macromolecules may be prepared by any suitable method, for example by reacting a cabazitaxel-containing precursor with a dendrimeric intermediate already containing a PEG group to introduce the pharmaceutically active agent, by reacting a PEG-containing precursor with a dendrimeric intermediate already containing a cabazitaxel residue, or by reacting an intermediate comprising the residue of a lysine group, a cabazitaxel residue and a PEG group with a dendrimeric intermediate.

Accordingly, there is provided a process for producing a macromolecule as defined herein, comprising:
a) reacting a cabazitaxel intermediate which is:

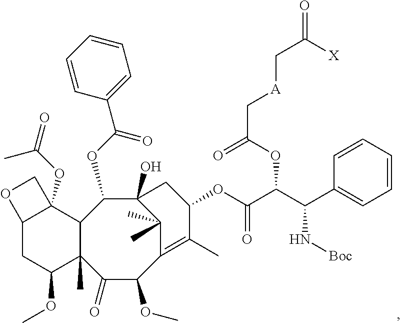

wherein A is —O—, —S—, or —NMe-; X is —OH or a leaving group, or wherein X together with the C(O) group to which it is attached forms a carboxylate salt;
with a dendrimeric intermediate which comprises:
i) a core unit (C); and
ii) building units (BU), each building unit being a lysine residue or an analogue thereof;
wherein the core unit is covalently attached to two building units via amide linkages, each amide linkage being formed between a nitrogen atom present in the core unit and the carbon atom of an acyl group present in a building unit;
the dendrimer being a five generation building unit dendrimer;
wherein building units of different generations are covalently attached to one another via amide linkages formed between a nitrogen atom present in one building unit and the carbon atom of an acyl group present in another building unit;
the macromolecule further comprising:
a plurality of second terminal groups (T2) each comprising a PEG group;
wherein at least one third of the nitrogen atoms present in the outer building units are each covalently attached to a second terminal group;
and wherein at least one third of the nitrogen atoms present in the outer building units are unsubstituted and available for reaction with the first intermediate;
or a salt thereof;
under amide coupling conditions;
or
b) reacting a PEG intermediate which is:

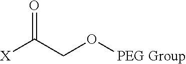

wherein PEG Group is a PEG-containing group, and
X is —OH or a leaving group, or wherein X together with the C(O) group to which it is attached forms a carboxylate salt;
with a dendrimeric intermediate which comprises:
i) a core unit (C); and
ii) building units (BU), each building unit being a lysine residue or an analogue thereof;
wherein the core unit is covalently attached to two building units via amide linkages, each amide linkage being formed between a nitrogen atom present in the core unit and the carbon atom of an acyl group present in a building unit;
the macromolecule being a five generation building unit dendrimer;
wherein building units of different generations are covalently attached to one another via amide linkages formed between a nitrogen atom present in one building unit and the carbon atom of an acyl group present in another building unit;
the macromolecule further comprising:
a plurality of first terminal groups (T1) each comprising a cabazitazel residue covalently attached to a diglycolyl, dithioacetyl or methyliminodiacetyl linker group;
wherein at least one third of the nitrogen atoms present in the outer building units are each covalently attached to a first terminal group;
and wherein at least one third of the nitrogen atoms present in the outer building units are unsubstituted;
or a salt thereof;
under amide coupling conditions;
or
c) reacting a surface unit intermediate which is:

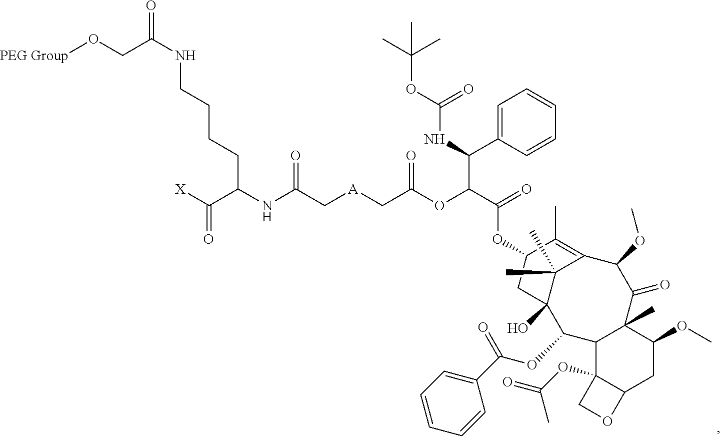

wherein A is —O—, —S— or —NMe-; PEG Group is a PEG-containing group, and
X is —OH or a leaving group, or wherein X together with the C(O) group to which it is attached forms a carboxylate salt;
with a dendrimeric intermediate comprising:
i) a core unit (C); and
ii) building units (BU), each building unit being a lysine residue or an analogue thereof;
wherein the core unit is covalently attached to two building units via amide linkages, each amide linkage being formed between a nitrogen atom present in the core unit and the carbon atom of an acyl group present in a building unit;
the dendrimeric intermediate being a four generation building unit dendrimeric intermediate;
wherein building units of different generations are covalently attached to one another via amide linkages formed between a nitrogen atom present in one building unit and the carbon atom of an acyl group present in another building unit;
and wherein nitrogen atoms present in the outer building units of the dendrimeric intermediate are unsubstituted;
or a salt thereof;
under amide coupling conditions.

Process variants a), b) and c) involve formation of amide bonds by reaction of —C(O)X groups with amine groups present in the dendrimeric intermediates. Any suitable amide formation conditions may be used. Examples of typical conditions include the use of a suitable solvent (for example dimethylformamide) optionally a suitable base, and at a suitable temperature (for example ambient temperature, e.g. in the range of from 15 to 30° C.). Where X is a leaving group, any suitable leaving group may be used, for example an activated ester. Where X is an —OH group or where X together with the C(O) group to which it is attached forms a carboxylate salt, the group will typically be converted to a suitable leaving group prior to reaction with a dendrimeric intermediate, for example by use of a suitable amide coupling reagent such as PyBOP.

Any suitable isolation and/or purification technique may be utilized, for example the dendrimer may be obtained by dissolution in a suitable solvent (e.g. THF) and precipitation by addition into an antisolvent (e.g. MTBE).

The cabazitaxel intermediate used in variant a) may itself be obtained, for example, by reaction of cabazitaxel with diglycolic anhydride or thiodiglycolic/thiodiacetic anhydride, or with methyliminodiacetic acid and a coupling agent agent such as EDCI and DMAP, for example in the presence of a suitable solvent such as dichloromethane, and for example in the presence of a suitable base such as triethylamine.

The surface unit intermediate used in variant c) may itself be obtained, for example, by:
i) reacting a PEG intermediate which is:

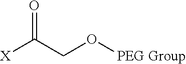

wherein PEG Group is a PEG-containing group, and
X is —OH or a leaving group, or wherein X together with the C(O) group to which it is attached forms a carboxylate salt;
with

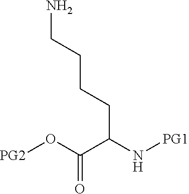

wherein PG1 is an amine protecting group (such as a Boc or Cbz group), and PG2 is an acid protecting group (such as a methyl or benzyl ester);
ii) deprotecting PG1;
iii) reacting the product of step ii) with a cabazitaxel intermediate which is:

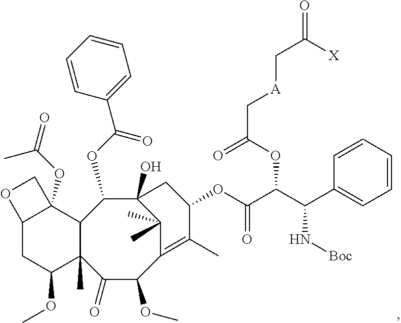

wherein A is —O—, —S—, or —NMe-; X is —OH or a leaving group, or wherein X together with the C(O) group to which it is attached forms a carboxylate salt; and
iv) deprotecting PG2.

The dendrimeric intermediate used in variant a) may itself be obtained by, for example, a sequential process involving:
i) reaction of a core unit (C) containing amino groups, with building units which are protected lysines or analogues thereof, which contain a —C(O)X group, wherein X is —OH or a leaving group or —CO(X) forms a carboxylate salt, and in which the amino groups present in the lysines or analogues thereof are protected, to form amide linkages between the core unit and building units;
ii) deprotecting protecting groups present on the building units;
iii) reacting free amino groups present on the building units with further building units which are protected lysines or analogues thereof, which contain a —C(O)X group, wherein X is —OH or a leaving group or —CO(X) forms a carboxylate salt, and in which the amino groups present in the lysines or analogues thereof are protected, to form amide linkages between the different generations of building units;
iv) deprotecting protecting groups present on the building units;
v) repeating steps iii) and iv) until a four generation building unit is produced;
vi) reacting free amino groups present on the building units with

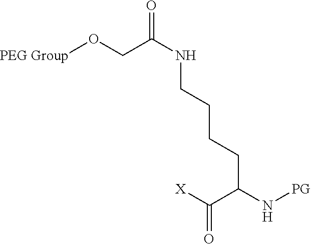

wherein PG is a protecting group, and wherein X is —OH or a leaving group, or wherein X together with the C(O) group to which it is attached forms a carboxylate salt, to form amide linkages therebetween; and
vii) deprotecting the protecting groups PG.

Alternatively, the dendrimeric intermediate used in variant a) may be obtained, for example, by carrying out steps i) to v) as described above, and:
vi) reacting free amino groups present on the building units with further building units which are protected lysines or analogues thereof, which contain a —C(O)X group, wherein X is —OH or a leaving group or —CO(X) forms a carboxylate salt, and in which the amino groups present in the lysines or analogues thereof are orthogonally protected, to form amide linkages between the different generations of building units;
vii) deprotecting a first set of amino protecting groups;
viii) reacting free amino groups present on the building units with

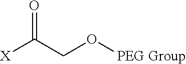

wherein PEG Group is a PEG-containing group, and X is —OH or a leaving group, or wherein X together with the C(O) group to which it is attached forms a carboxylate salt; and
ix) deprotecting a second set of amino protecting groups.

The dendrimeric intermediate used in variant b) may itself be obtained, for example, by carrying out steps i) to v) as described above in relation to variant a), and:
vi) reacting free amino groups present on the building units with

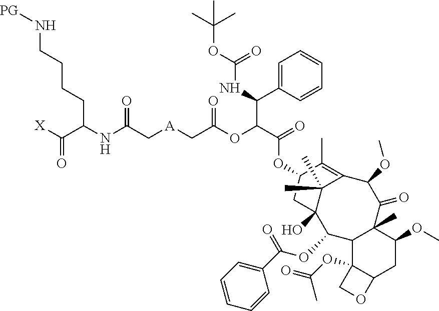

wherein A is —O—, —S—, or —NMe-, PG is a protecting group, and wherein X is —OH or a leaving group, or wherein X together with the C(O) group to which it is attached forms a carboxylate salt, to form amide linkages therebetween; and
vii) deprotecting the protecting groups PG.

Alternatively, the dendrimeric intermediate used in variant b) may be obtained, for example, by carrying out steps i) to v) as described above, and:
vi) reacting free amino groups present on the building units with further building units which are protected lysines or analogues thereof, which contain a —C(O)X group, wherein X is —OH or a leaving group or —CO(X) forms a carboxylate salt, and in which the amino groups present in the lysines or analogues thereof are orthogonally protected, to form amide linkages between the different generations of building units;
vii) deprotecting a first set of amino protecting groups;
viii) reacting free amino groups present on the building units with

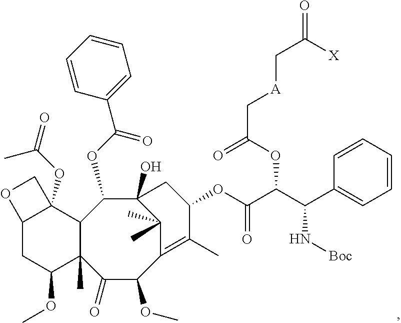

wherein A is —O—, —S— or —NMe-, X is —OH or a leaving group, or wherein X together with the C(O) group to which it is attached forms a carboxylate salt; and
ix) deprotecting a second set of amino protecting groups.

The dendrimeric intermediate used in variant c) may itself be obtained, for example, by carrying out steps i) to v) as described above in relation to variant a).

The present disclosure also provides synthetic intermediates useful in producing the macromolecules. Accordingly, there is also provided an intermediate for producing a macromolecule which is

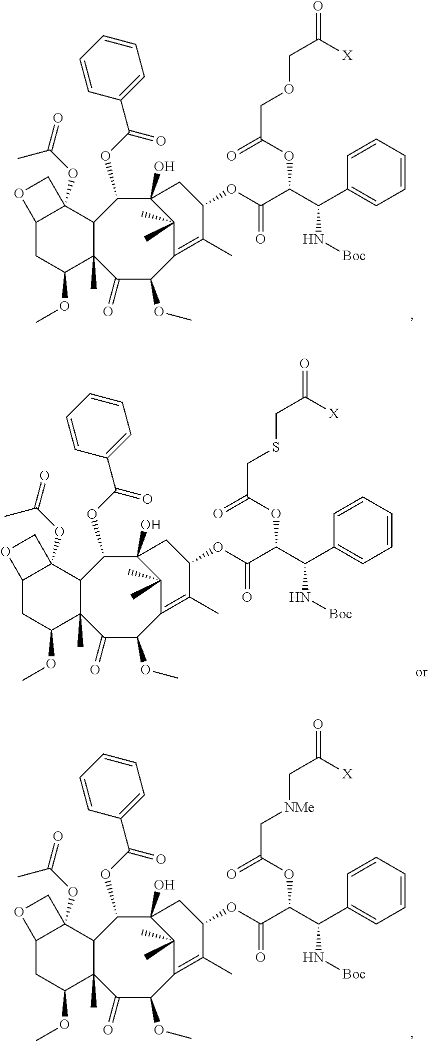

wherein X is —OH or a leaving group, or wherein X together with the C(O) group to which it is attached forms a carboxylate salt. Such an intermediate may be produced, for example, as described above.

There is also provided an intermediate for producing a macromolecule which is

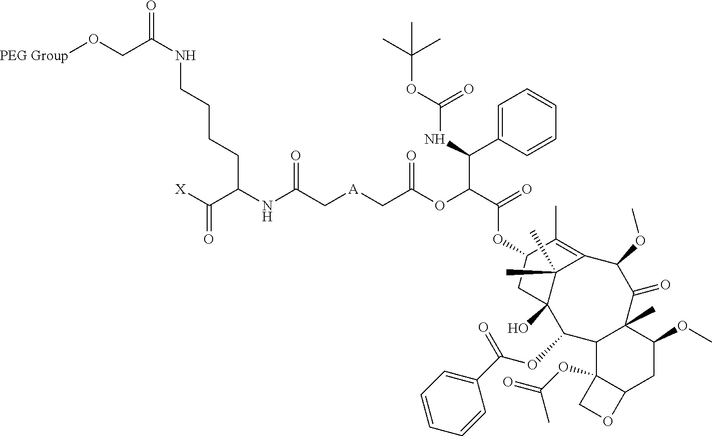

wherein A is —O—, —S— or —NMe-,
PEG Group is a PEG-containing group, and
X is —OH or a leaving group, or wherein X together with the C(O) group to which it is attached forms a carboxylate salt. Such an intermediate may be produced, for example, as described above.

### Compositions Comprising the Macromolecule

While it is possible that the macromolecules of the invention may be administered as a neat chemical, in particular embodiments, the macromolecule is presented as a pharmaceutical composition.

It will be appreciated that there may be some variation in the molecular composition between the dendrimers present in a given composition, as a result of the nature of the synthetic process for producing the dendrimers. For example, as discussed above one or more synthetic steps used to produce a dendrimer may not proceed fully to completion, which may result in the presence of dendrimers which do not all comprise the same number of first terminal groups or second terminal groups, or which contain incomplete generations of building units.

Accordingly, there is provided a composition comprising a plurality of dendrimers or pharmaceutically acceptable salts thereof, wherein the dendrimers are as defined herein, the mean number of first terminal groups per dendrimer in the composition is in the range of from 24 to 32, and the mean number of second terminal groups per dendrimer in the composition is in the range of from 24 to 32. In some embodiments, the mean number of first terminal groups per dendrimer is in the range of from 26 to 32, and wherein the mean number of second terminal groups per dendrimer is in the range of from 28 to 32. In some embodiments, the mean number of first terminal groups per dendrimer is in the range of from 28 to 32, or in the range of from 29 to 31. In some embodiments, the mean number of second terminal groups per dendrimer is in the range of from 29 to 31. In some embodiments, the composition is a pharmaceutical composition, and wherein the composition comprises a pharmaceutically acceptable excipient.

In some embodiments, at least 50%, at least 60%, at least 70%, at least 80%, at least 90%, or at least 95% of the dendrimers contain at least 24 first terminal groups. In some embodiments, at least 50%, at least 60%, at least 70%, at least 80%, at least 90%, or at least 95% of the dendrimers contain at least 26 first terminal groups. In some embodiments, at least 50%, at least 60%, at least 70%, at least 80%, at least 90%, or at least 95% of the dendrimers contain at least 28 first terminal groups.

In some embodiments, at least 50%, at least 60%, at least 70%, at least 80%, at least 90%, or at least 95% of the dendrimers contain at least 28 second terminal groups. In some embodiments, at least 50%, at least 60%, at least 70%, at least 80%, at least 90%, or at least 95% of the dendrimers contain at least 29 second terminal groups.

In some embodiments, at least 50%, at least 60%, at least 70%, at least 80%, at least 90%, or at least 95% of the dendrimers contain at least 24 first terminal groups and at least 28 second terminal groups. In some embodiments, at least 50%, at least 60%, at least 70%, at least 80%, at least 90%, or at least 95% of the dendrimers contain at least 26 first terminal groups and at least 29 second terminal groups.

The invention provides pharmaceutical formulations or compositions, both for veterinary and for human medical use, which comprise one or more macromolecules of the invention or a pharmaceutically acceptable salt thereof, with one or more pharmaceutically acceptable carriers, and optionally any other therapeutic ingredients, stabilisers, or the like. The carrier(s) must be pharmaceutically acceptable in the sense of being compatible with the other ingredients of the formulation and not unduly deleterious to the recipient thereof. The compositions of the invention may also include polymeric excipients/additives or carriers, e.g., polyvinylpyrrolidones, derivatised celluloses such as hydroxymethylcellulose, hydroxyethylcellulose, and hydroxypropylmethylcellulose, Ficolls (a polymeric sugar), hydroxyethylstarch (HES), dextrates (e.g., cyclodextrins, such as 2-hydroxypropyl-β-cyclodextrin and sulfobutylether-β-cyclodextrin), polyethylene glycols, and pectin. The compositions may further include diluents, buffers, binders, disintegrants, thickeners, lubricants, preservatives (including antioxidants), flavoring agents, taste-masking agents, inorganic salts (e.g., sodium chloride), antimicrobial agents (e.g., benzalkonium chloride), sweeteners, antistatic agents, sorbitan esters, lipids (e.g., phospholipids such as lecithin and other phosphatidylcholines, phosphatidylethanolamines, fatty acids and fatty esters, steroids (e.g., cholesterol)), and chelating agents (e.g., EDTA, zinc and other such suitable cations). Other pharmaceutical excipients and/or additives suitable for use in the compositions according to the invention are listed in “Remington: The Science & Practice of Pharmacy”, 19.sup.th ed., Williams & Williams, (1995), and in the “Physician's Desk Reference”, 52.sup.nd ed., Medical Economics, Montvale, N.J. (1998), and in “Handbook of Pharmaceutical Excipients”, Third Ed., Ed. A. H. Kibbe, Pharmaceutical Press, 2000.

The macromolecule may also be formulated in the presence of an appropriate albumin protein such as human serum albumin. Albumin carries nutrients around the body and may bind to the macromolecule and carry it to its site of action.

The macromolecules of the invention may be formulated in compositions including those suitable for oral, rectal, topical, nasal, inhalation to the lung, by aerosol, ophthalmic, or parenteral (including intraperitoneal, intravenous, subcutaneous, or intramuscular injection) administration. The compositions may conveniently be presented in unit dosage form and may be prepared by any of the methods well known in the art of pharmacy. All methods include the step of bringing the macromolecule into association with a carrier that constitutes one or more accessory ingredients.

In general, the compositions are prepared by bringing the macromolecule into association with a liquid carrier to form a solution or a suspension, or alternatively, bring the macromolecule into association with formulation components suitable for forming a solid, optionally a particulate product, and then, if warranted, shaping the product into a desired delivery form. Solid formulations of the invention, when particulate, will typically comprise particles with sizes ranging from about 1 nanometer to about 500 microns. In general, for solid formulations intended for intravenous administration, particles will typically range from about 1 nm to about 10 microns in diameter. The composition may contain macromolecule of the invention that are nanoparticulate having a particulate diameter of below 1000 nm, for example, between 5 and 1000 nm, especially 5 and 500 nm, more especially 5 to 400 nm, such as 5 to 50 nm and especially between 5 and 20 nm. In particular embodiments, the composition contains macromolecules with a mean size of between 5 and 20 nm. In some embodiments, the macromolecule is polydispersed in the composition, with PDI of between 1.01 and 1.8, especially between 1.01 and 1.5, and more especially between 1.01 and 1.2. In particular embodiments, the macromolecule is monodispersed in the composition. Particularly preferred are sterile, lyophilized compositions that are reconstituted in an aqueous vehicle prior to injection.

Compositions of the present invention suitable for oral administration may be presented as discrete units such as capsules, cachets, tablets, lozenges, and the like, each containing a predetermined amount of the active agent as a powder or granules; or a suspension in an aqueous liquor or non-aqueous liquid such as a syrup, an elixir, an emulsion, a draught, and the like.

A tablet may be made by compression or molding, optionally with one or more accessory ingredients. Compressed tablets may be prepared by compressing in a suitable machine, with the active compound being in a free-flowing form such as a powder or granules which is optionally mixed with a binder, disintegrant, lubricant, inert diluent, surface active agent or dispersing agent. Molded tablets comprised with a suitable carrier may be made by molding in a suitable machine.

A syrup may be made by adding the active compound to a concentrated aqueous solution of a sugar, for example sucrose, to which may also be added any accessory ingredient(s). Such accessory ingredients may include flavorings, suitable preservatives, an agent to retard crystallization of the sugar, and an agent to increase the solubility of any other ingredient, such as polyhydric alcohol, for example, glycerol or sorbitol.

In some preferred embodiments, the composition is formulated for parenteral delivery. For example, in one embodiment, the formulation may be a sterile, lyophilized composition that is suitable for reconstitution in an aqueous vehicle prior to injection.

Formulations suitable for parenteral administration conveniently comprise a sterile aqueous preparation of the macromolecule, which can be formulated to be isotonic with the blood of the recipient.

Nasal spray formulations comprise purified aqueous solutions of the active agent with preservative agents and isotonic agents. Such formulations are preferably adjusted to a pH and isotonic state compatible with the nasal mucous membranes.

Formulations for rectal administration may be presented as a suppository with a suitable carrier such as cocoa butter, or hydrogenated fats or hydrogenated fatty carboxylic acids.

Ophthalmic formulations are prepared by a similar method to the nasal spray, except that the pH and isotonic factors are preferably adjusted to match that of the eye.

Topical formulations comprise the active compound dissolved or suspended in one or more media such as mineral oil, petroleum, polyhydroxy alcohols or other bases used for topical formulations. The addition of other accessory ingredients as noted above may be desirable.

Pharmaceutical formulations are also provided which are suitable for administration as an aerosol, by inhalation. These formulations comprise a solution or suspension of the desired macromolecule or a salt thereof. The desired formulation may be placed in a small chamber and nebulized. Nebulization may be accomplished by compressed air or by ultrasonic energy to form a plurality of liquid droplets or solid particles comprising the macromolecules or salts thereof.

Often drugs are co-administered with other drugs in combination therapy, especially during chemotherapy. The macromolecules of the invention may therefore be administered as combination therapies. For example, when the pharmaceutically active agent is docetaxel, the macromolecule may be administered with doxorubicin, cyclophosphamide or capecitabine. Not only can the macromolecules be administered with other chemotherapy drugs but may also be administered in combination with other medications such as corticosteroids, anti-histamines, analgesics and drugs that aid in recovery or protect from hematotoxicity, for example, cytokines.

In some embodiments, particularly with oncology drugs, the composition is formulated for parenteral infusion as part of a chemotherapy regimen. In these embodiments, the compositions are substantially free or entirely free of solubilisation excipients, especially solubilisation excipients such as Cremophor and polysorbate 80. In particular embodiments, the pharmaceutically active agent is selected from docetaxel or paclitaxel and the formulation is substantially free or entirely free of solubilisation excipients such as Cremophor and polysorbate 80. By removing the solubilisation excipient the composition of dendrimer is less likely to cause side effects such as acute or delayed hypersensitivity including life-threatening anaphylaxis and/or severe fluid retention.

In some embodiments, the macromolecule is formulated for transdermal delivery such as an ointment, a lotion or in a transdermal patch or use of microneedle technology. High drug loading and aqueous solubility allows small volumes to carry sufficient drug for patch and microneedle technologies to provide a therapeutically effective amount. Such formulations are particularly suitable for delivery of testosterone.

The macromolecules of the invention may also be used to provide controlled-release of the pharmaceutically active agents and/or slow-release formulations.

In slow-release formulations, the formulation ingredients are selected to release the macromolecule from the formulation over a prolonged period of time, such as days, weeks or months. This type of formulation includes transdermal patches or in implantable devices that may be deposited subcutaneously or by injection intravenously, subcutaneously, intramuscularly, intraepidurally or intracranially.

In controlled-release formulations, the diacid linker is selected to release a majority of its pharmaceutically active agent in a given time window. For example, when the time taken for a majority of the macromolecule to accumulate in a target organ, tissue or tumor is known, the linker may be selected to release a majority of its pharmaceutically active agent after the time to accumulate has elapsed. This can allow a high drug load to be delivered at a given time point at the site where its action is required. Alternatively, the linker is selected to release the pharmaceutically active agent at a therapeutic level over a prolonged period of time.

In some embodiments, the formulation may have multiple controlled-release characteristics. For example, the formulation comprises macromolecules in which the drug is attached through different linkers allowing an initial burst of fast-released drug followed by slower release at low but constant therapeutic levels over a prolonged period of time.

In some embodiments, the formulation may have both slow-release and controlled-release characteristics. For example, the formulation ingredients may be selected to release the macromolecule over a prolonged period of time and the linker is selected to deliver a constant low therapeutic level of pharmaceutically active agent.

In some embodiments, the pharmaceutically active agent is attached to the same molecule through different linkers. In other embodiments, each drug-linker combination is attached to different macromolecules in the same formulation.

### Methods of Use

The macromolecule of the invention may be used to treat or prevent any disease, disorder or symptom that the unmodified pharmaceutically active agent can be used to treat or prevent.

In some embodiments, where the pharmaceutically active agent is an oncology drug, the macromolecule is used in a method of treating or preventing cancer, or suppressing the growth of a tumor. In particular embodiments, the drug is selected from docetaxel, camptothecin, topotecan, irinotecan and gemcitabine, especially docetaxel.

In some embodiments, the cancer is a blood borne cancer such as leukaemia or lymphoma. In other embodiments, the cancer is a solid tumor. The solid tumor may be a primary or a metastatic tumor. Exemplary solid tumors include tumors of the breast, lung especially non-small cell lung cancer, colon, stomach, kidney, brain, head and neck especially squamous cell carcinoma of the head and neck, thyroid, ovary, testes, liver, melanoma, prostate especially androgen-independent (hormone refractory) prostate cancer, neuroblastoma and gastric adenocarcinoma including adenocarcinoma of the gastroesophageal junction.

In some embodiments, the cancer, is selected from the group consisting of breast cancer, ovarian cancer (e.g. recurrent ovarian cancer), testicular cancer (e.g. cis-platin-resistant germ cell cancer), prostate cancer (e.g. bone metastatic prostate cancer, prostatic neoplasms, hormone-refractory prostate cancer, castration resistant prostate cancer, advanced prostate cancer), dedifferentiated liposarcoma, urothelial carcinoma of the urinary bladder (e.g. urothelium transitional cell carcinoma (TCCU)), adrenocortical carcinoma, brain cancer (e.g. recurrent malignant glioma), AML (acute myeloid leukemia) and CLL (chronic lymphocytic leukemia). In some embodiments, the cancer is prostate cancer or breast cancer. In some embodiments the cancer is prostate cancer, for example hormone-refractory prostate cancer, or for example metastatic castration-resistant prostate cancer (mCRPC). In some embodiments the cancer is breast cancer.

Oncology drugs often have significant side effects that are due to off-target toxicity such as hematologic toxicity, neurological toxicity, cardiotoxicity, hepatotoxicity, nephrotoxicity, ototoxicity and encephalotoxicity. For example, taxanes such as docetaxel may cause the following adverse effects: infections, neutropenia, anemia, febrile neutropenia, hypersensitivity, thrombocytopenia, myelotoxicity, myelosuppression, neuropathy, dysgeusia, dyspnea, constipation, anorexia, nail disorders, fluid retention, asthenia, pain, nausea, diarrhea, vomiting, fatigue, non-specific neuro cognitive problems, vertigo, encephalopathy, mucositis, alopecia, skin reactions and myalgia.

Furthermore, solubilisation excipients required to formulate the oncology drugs may cause anaphylaxis, fluid retention and hypersensitivity. Premedication with corticosteroids, anti-histamines, cytokines and/or analgesics may also be required, each having their own side effects. The macromolecules of the present invention have high drug loading, controlled-release, may passively target a particular tissue and improve solubility allowing a reduction of side effects associated with the oncology drug, the formulation of the drug without solubilisation excipients and administration without or with reduced premedication.

In another aspect of the invention, there is provided a method of reducing the side effects of an oncology drug or the side-effects relating to the formulation of an oncology drug comprising administering an effective amount of the macromolecule of the present invention to a subject, wherein the oncology drug is the pharmaceutically active agent of the first terminal group.

In yet another aspect of the invention, there is provided a method of reducing hypersensitivity during chemotherapy comprising administering an effective amount of the macromolecule of the invention to a subject.

Therapeutic regimens for cancer treatment often involve a cyclic therapy where an oncology drug is administered once every two to four weeks. Often the drug is administered by infusion over 3 to 24 hours. In some cases to reduce the side effects of the drugs, or the risk of hypersensitivity, especially anaphylaxis from the formulation of the drug; premedication is required and its administration may be required up to 6 hours prior to treatment with the oncology drug. Such complex therapeutic regimens are time consuming and require the patient to remain in hospital from several hours to 2 days. The severe side effects may also limit the dose of oncology drug used and/or the number of cycles of therapy that can be administered and therefore in some cases efficacy of the therapy is diminished.

In the present invention, the macromolecule comprising the oncology drug reduces side effects associated with the drug as it passively accumulates at the tumor site or is directed to the tumor site by an appropriate targeting agent and release of the drug from the dendrimer is controlled.

The solubility of the macromolecules in aqueous solution allows them to be formulated without harmful solubilisation excipients thereby reducing side effects of the formulation and in some cases eliminating the need for premedication.

Furthermore, the macromolecules of the present invention need not be administered by prolonged infusion. In some embodiments, they may be administered by fast-infusion, for example, in less than 3 hours, including 2.5 hours, 2 hours, 1.5 hours, 1 hour or 30 minutes. In some embodiments, the macromolecule or formulation of macromolecule may be administered as a bolus, for example, in 5 seconds to 5 minutes.

The macromolecules of the present invention may also allow the dose of the pharmaceutically active agent to be increased compared to the pharmaceutically active agent being administered alone. In another aspect of the invention there is provided a method of increasing the dose of a pharmaceutically active agent comprising administering the macromolecule of the present invention wherein the first terminal group is the pharmaceutically active agent. In particular embodiments, the maximum tolerated dose is increased at least two fold compared to the pharmaceutically active agent when administered alone.

In particular embodiments of these aspects, the formulation of the macromolecule used in administration is substantially free of solubilisation excipients such as polyethoxylated caster oil (Cremophor EL) and polysorbate 80.

In some embodiments where the pharmaceutically active agent is testosterone or dihydrotestosterone and the macromolecule is used in a method of treating or preventing a disease or disorder associated with low testosterone levels.

Low testosterone levels may result from a number of conditions. For example, the organs that produce testosterone (testis, ovaries) do not produce enough testosterone (primary hypogonadism), the pituitary gland and its ability to regulate testosterone production is not working properly (secondary hypogonadism) or the hypothalamus may not be regulating hormone production correctly (tertiary hypogonadism).

Common causes of primary hypogonadism include undescended testicles, injury to the scrotum, cancer therapy, aging, mumps orchitis, chromosomal abnormalities, ovary conditions such as premature ovary failure or removal of both ovaries. Causes of secondary and tertiary hypogonadism include damage to the pituitary gland from tumors or treatment of nearby tumors, hypothalamus malformations such as in Kellman's syndrome, compromised blood flow to the pituitary gland or hypothalamus, inflammation caused by HIV/AIDS, inflammation from tuberculosis or sarcoides and the illegal use of anabolic steroids in body building.

It should also be noted that obesity can also be a cause of low testosterone levels as obesity significantly enhances the conversion of testosterone to estrogen, a process that occurs predominantly in fat cells.

Symptoms of low testosterone include changes in mood (depression, fatigue, anger), decreased body hair, decreased mineral bone density (increased risk of osteoporosis), decreased lean body mass and muscle strength, decreased libido and erectile dysfunction, increased abdominal fat, rudimentary breast development in men and low or no sperm in semen.

An “effective amount” means an amount necessary at least partly to attain the desired response, or to delay the onset or inhibit progression or halt altogether, the onset or progression of a particular condition being treated. The amount varies depending upon the disease being treated, the health and physical condition of the individual to be treated, the taxonomic group of individual to be treated, the degree of protection desired, the formulation of the composition, the assessment of the medical situation, and other relevant factors. It is expected that the amount will fall in a relatively broad range that can be determined through routine trials. An effective amount in relation to a human patient, for example, may lie in the range of about 0.1 ng per kg of body weight to 1 g per kg of body weight per dosage. In a particular embodiment the dosage is in the range of 1 μg to 1 g per kg of body weight per dosage, such as is in the range of 1 mg to 1 g per kg of body weight per dosage. In one embodiment, the dosage is in the range of 1 mg to 500 mg per kg of body weight per dosage. In another embodiment, the dosage is in the range of 1 mg to 250 mg per kg of body weight per dosage. In yet another embodiment, the dosage is in the range of 1 mg to 100 mg per kg of body weight per dosage, such as up to 50 mg per kg of body weight per dosage. In yet another embodiment, the dosage is in the range of 1 μg to 1 mg per kg of body weight per dosage. Dosage regimes may be adjusted to provide the optimum therapeutic response. For example, several divided doses may be administered daily, weekly, monthly or other suitable time intervals, or the dose may be proportionally reduced as indicated by the exigencies of the situation.

In some embodiments the macromolecule is administered intravenously, intraarterially, intrapulmonarily, orally, by inhalation, intravesicularlly, intramuscularly, intratracheally, subcutaneously, intraocularly, intrathecally or transdermally.

In some embodiments the macromolecule is administered as a bolus or by fast infusion, especially as a bolus.

In another aspect of the invention there is provided the use of a macromolecule of the invention in the manufacture of a medicament for treating or suppressing the growth of cancer, reducing the toxicity of an oncology drug or a formulation of an oncology drug, reducing side effects associated with an oncology drug or a formulation of an oncology drug or reducing hypersensitivity upon treatment with an oncology drug; wherein the pharmaceutically active agent of the first terminal group is an oncology drug.

In yet another aspect of the invention there is provided a use of a macromolecule of the invention in the manufacture of a medicament for treating or preventing a disease or disorder related to low testosterone levels; wherein the pharmaceutically active agent of the first terminal group is testosterone.

Drugs are often co-administered with other drugs in combination therapy, especially during chemotherapy. Accordingly, in some embodiments the macromolecule is administered in combination with one or more further pharmaceutically active agents, for example one or more further anti-cancer agents. The macromolecule and the one or more further pharmaceutically active agents may be administered simultaneously, subsequently or separately. For example, they may be administered as part of the same composition, or by administration of separate compositions. The one or more further pharmaceutically active agents may for example be anti-cancer agents for therapy of prostate cancer or breast cancer. Examples of further pharmaceutically active agents include chemotherapeutic and cytotoxic agents, checkpoint inhibitors, and antibody therapies. Another pharmaceutically active agent for use in combination with the dendrimers is prednisone. Examples of further pharmaceutically active agents include docetaxel, clarithromycin, vinflunine, bavituximab and tocotrienol. Additional examples of further pharmaceutically active agents include corticosteroids (such as dexamethasone), anti-histamines (such as dexchlorpheniramine or diphenhydramine), H2 antagonists (such as ranitidine), analgesics, antiemetics, and drugs that aid in recovery from and/or protect from hematotoxicity, such as cytokines. It will be appreciated that a therapeutically effective amount refers to a macromolecule being administered in an amount sufficient to alleviate or prevent to some extent one or more of the symptoms of the disorder or condition being treated. A therapeutically effective amount of macromolecule may be referred to based on, for example, the amount of dendrimer administered. Alternatively, it may be determined based on the amount of active agent (e.g. cabazitazel) which the macromolecule is theoretically capable of delivering, e.g. based on the loading of cabazitaxel on the macromolecule.

In some embodiments, the amount of macromolecule administered is sufficient to deliver between 5 and 100 mg of active agent/m2, between 5 and 50 mg of active agent/m2, between 5 and 40 mg of active agent/m2, between 5 and 30 mg of active agent/m2, between 5 and 25 mg of active agent/m2, between 5 and 20 mg of active agent/m2, between 10 and 50 mg of active agent/m2, between 20 to 40 mg of active agent/m2 between 15 and 35 mg of active agent/m2, between 10 and 20 mg/m2, between 20 and 30 mg/m2, or between 25 and 35 mg of active agent/m2. For example, cabazitaxel is indicated for use at 20-25 mg/m2 and similar or slightly higher doses of active agent have been demonstrated to be effective for the dendrimer in the comparative mouse studies below. A dose of active agent of 10 mg/kg in a mouse should be approximately equivalent to a human dose of 30 mg/m2 (FDA guidance 2005). (To convert human mg/kg dose to mg/m2, the figure may be multiplied by 37, FDA guidance 2005).

In some embodiments, the pharmaceutically active agent is cabazitaxel and the amount of macromolecule administered delivers an amount of cabazitaxel to a patient which is in the range of from 0.5 to 3 times the amount of cabazitaxel delivered upon administration of 20-25 mg/m2 free cabazitaxel. In some embodiments, the amount of macromolecule administered delivers an amount of cabazitaxel to a patient which is in the range of from 1 to 2 times the amount of cabazitaxel delivered upon administration of 20-25 mg/m2 free cabazitaxel. In some embodiments, the amount of macromolecule administered delivers an amount of cabazitaxel to a patient which is in the range of from 0.5 to 1.5 times the amount of cabazitaxel delivered upon administration of 20-25 mg/m2 free cabazitaxel. In some embodiments, the amount of macromolecule administered delivers an amount of cabazitaxel to a patient which is in the range of from 0.8 to 1.2 times the amount of cabazitaxel delivered upon administration of 20-25 mg/m2 free cabazitaxel. In some embodiments, the amount of macromolecule administered delivers substantially an equivalent amount of cabazitaxel to that delivered on administration of an authorised dosage of free cabazitaxel (e.g. Jevtana®). For example, as discussed above, recommended dosage levels for cabazitaxel are 20-25 mg/m2. In some embodiments, the amount of macromolecule administered is capable of delivering an amount of cabazitaxel to a patient substantially equivalent to administration of 20-25 mg/m2 free cabazitaxel. The amount of macromolecule administered may for example be determined with reference to the amount of cabazitaxel which the macromolecule is capable of delivering (i.e. cabazitaxel loading).

In some embodiments, a therapeutically effective amount of the macromolecule is administered to a subject in need thereof at a predetermined frequency. In some embodiments, the macromolecule is administered to a subject in need thereof according to a dosage regimen in which the macromolecule is administered once per one to four weeks. In some embodiments, the macromolecule is administered to a subject in need thereof according to a dosage regimen in which the macromolecule is administered once per three to four weeks.

It has been surprisingly found that a macromolecule of the present disclosure has increased efficacy in comparison to the direct administration of the free drug. As used herein, the term “free” refers to a drug, e.g., cabazitaxel, which has not been previously conjugated to a dendrimer. For example, the direct administration of free cabazitaxel refers to the direct administration of cabazitaxel molecules that are not administered as being conjugated to a dendrimer. An example of such a therapy is Jevtana®. As used herein, the terms “unconjugated” and “released” refer to a drug, e.g. cabazitaxel, which has dissociated or been cleaved from a dendrimer. This dissociation or cleaving may occur in vivo following administration of the drug-dendrimer conjugate. Specifically, in some embodiments, the macromolecules of the present disclosure provide increased therapeutic drug exposure (AUC), a lower maximal concentration (Cmax), an increased half-life (t1/2), reduced Tmax and/or reduced toxicity, in comparison to administration of an equivalent amount of the unconjugated dug.

Accordingly, in some embodiments, the pharmaceutically active agent is cabazitaxel and administration of the macromolecule provides at least 1.5 times the therapeutic drug exposure (AUC) of cabazitaxel, in comparison to the direct administration of an equivalent dose of free cabazitaxel. An equivalent dose of free cabazitaxel is the equivalent amount of free cabazitaxel to the amount of cabazitaxel contained (loaded) in the dose of macromolecule to be administered. Oncology drugs often have significant side effects that are due to off-target toxicity such as hematologic toxicity, neurological toxicity, cardiotoxicity, hepatotoxicity, nephrotoxicity, ototoxicity and encephalotoxicity. For example, taxanes such as cabazitaxel may cause the following adverse effects: infections, neutropenia, anaemia, febrile neutropenia, hypersensitivity, thrombocytopenia, myelotoxicity, myelosuppression, neuropathy, dysgeusia, dyspnoea, constipation, anorexia, nail disorders, fluid retention, asthenia, pain, nausea, diarrhoea, vomiting, fatigue, non-specific neuro cognitive problems, vertigo, encephalopathy, mucositis, alopecia, skin reactions and myalgia.

In some embodiments, the pharmaceutically active agent is cabazitaxel and administration of the macromolecule provides reduced toxicity in comparison to administration of an equivalent dose of free cabazitaxel. The toxicity of a drug refers to the degree to which damage is caused to the organism, and is measured by its effect off target. In oncology, one such measurement of toxicity in animal models is weight loss, which determines the maximum tolerated dose (MTD). In humans toxicity is commonly determined by specified adverse events (AE), which typically identify the dose limiting toxicity. It will be appreciated that usually in oncology, there is a narrow therapeutic window and off-target toxicities are considered a normal side effect of killing tumour cells. It will also be appreciated that toxicity is commonly related to drug exposure (AUC), however, surprisingly in the present disclosure, the AUC for released unconjugated drug is increased compared to AUC following administration of equivalent amounts of free drug, while reducing toxicity or improving efficacy. In some embodiments, administration of the macromolecule provides reduced toxicity in comparison to administration of an equivalent dose of free cabazitaxel when used in a method of treatment of cancer, such as hormone-refractory prostate cancer, metastatic castration-resistant prostate cancer (mCRPC), or breast cancer.

Toxicity studies carried out with a macromolecule of the present disclosure indicate that the macromolecule is likely to induce less neutropenia, and therefore be less toxic in the clinic, compared with the administration of an equivalent dose of free cabazitaxel. Accordingly, in some embodiments, the pharmaceutically active agent is cabazitaxel and administration of the macromolecule provides reduced neutropenia in comparison to administration of an equivalent dose of free cabazitaxel. In some embodiments, administration of the macromolecule provides reduced neutropenia in comparison to administration of an equivalent dose of free cabazitaxel, when used in a method of treatment of cancer, such as hormone-refractory prostate cancer, metastatic castration-resistant prostate cancer (mCRPC), or breast cancer.

In some embodiments, the macromolecule provides a reduction in toxicity as measured by the number of patients having specified AE (eg infections (cystitis, upper respiratory tract, herpes zoster, candidiasis, sepsis, influenza, UTI) fever, neutropenia, anaemia, febrile neutropenia, thrombocytopenia, leukopenia, myelotoxicity, myelosuppression, neuropathy, hypersensitivity, dysgeusia, gastrointestinal toxicity, dyspnoea, cough, abdominal pain, constipation, anorexia, nail disorders, fluid retention, asthenia, pain, nausea, diarrhoea, vomiting, fatigue, non-specific neuro cognitive problems, headache, vertigo, back pain, arthralgia, encephalopathy, mucositis, alopecia, skin reactions and myalgia), by at least 10%, at least 20%, at least 30%, at least 40%, at least 50%, at least 60%, at least 70%, at least 80%, or at least 90%, in comparison to the direct administration of an equivalent dose of the free pharmaceutically active agent. In one example, the pharmaceutically active agent is cabazitaxel and administration of the macromolecule provides less than 95%, less than 90%, less than 80%, less than 70%, less than 60%, less than 50%, less than 40%, less than 30%, less than 20%, or less than 10% toxicity in comparison to the direct administration of an equivalent dose of free cabazitaxel.

The macromolecules of the present disclosure surprisingly achieve a sustained pharmacokinetic profile for unconjugated or released drug, resulting in a significantly increased AUC compared to an equivalent or normalised quantity of free drug. This sustained pharmacokinetic profile, and the associated increased AUC for released/unconjugated active agent indicates that the drug will be present in vivo at therapeutically effective levels for longer periods of time. It will be appreciated that exposure to the drug for a longer period of time is desirable as it may prolong the therapeutic effect of the drug and allow for reduced frequency of dosing. In some embodiments, the dendrimer provides increased therapeutic drug exposure/area under the curve (AUC) of total and/or unconjugated cabazitaxel in comparison to direct administration of an equivalent dose of free cabazitaxel. AUC is the area under the curve in a plot of drug concentration in blood plasma versus time. The AUC represents the total drug exposure over time. It would be appreciated that the AUC is normally proportional to the total amount of drug delivered to the body.

In some embodiments, the pharmaceutically active agent is cabazitaxel and the macromolecule achieves a more sustained in vivo pharmokinetic profile for concentration levels of released cabazitaxel, in comparison to the pharmacokinetic profile for concentration levels of cabazitaxel achieved on administration of an equivalent dose of free cabazitaxel.

In some embodiments, the pharmaceutically active agent is cabazitaxel and the macromolecule has increased therapeutic drug exposure (AUC) of unconjugated/released cabazitaxel in comparison to the direct administration of an equivalent dose of free cabazitaxel when used in a method of treatment, for example, in the treatment of cancer, such as hormone-refractory prostate cancer, metastatic castration-resistant prostate cancer (mCRPC), or breast cancer. In some embodiments, administration of the macromolecule provides at least 1.5 times, at least 2 times, at least 2.5 times, at least 3 times, at least 3.5 times, or at least 4 times, the therapeutic drug exposure (AUC) of cabazitaxel in comparison to the direct administration of an equivalent dose of free cabazitaxel. In some embodiments, administration of the macromolecule provides between 1.5 and 4 times, 1.7 and 3 times, or 1.8 and 2.5 times, the therapeutic drug exposure (AUC) of cabazitaxel in comparison to the direct administration of an equivalent dose of free cabazitaxel. In some embodiments, the amount of macromolecule administered is sufficient to provide released cabazitaxel exposure (AUC0-t) of about 200, about 400, about 450, about 500, about 550, about 600, about 750, about 1000, about 1200, or about 1250 ng·h/mL.

In addition to having a sustained in vivo pharmacokinetic profile providing comparatively high levels of exposure, the macromolecules also achieve comparatively low Cmax levels upon in vivo administration. In some embodiments, administration of the macromolecule provides a lower maximal concentration (Cmax) of unconjugated drug in comparison to direct administration of an equivalent dose of free drug. The maximal concentration (Cmax) of drug is the maximum (or peak) serum concentration that a drug achieves in a specified compartment or test area of the body after the drug has been administered and before the administration of a second dose. It will be appreciated that, whilst it is important to be able to dose a pharmaceutical agent at a level sufficient to achieve therapeutic concentration levels, if the maximum concentration levels reached are high, the risk of encountering off-target effects, side-effects and toxicity increase. This is particularly an issue for compounds which have a short half-life, since in such cases, in order to provide therapeutically effective levels of the active agent for a prolonged period of time, it may be necessary to increase the dose and thus the Cmax such that the likelihood of side-effects increases. Accordingly, it is highly desirable to be able to deliver a pharmaceutically active agent in a form which provides therapeutically effective levels for a sustained period of time, whilst at the same time avoiding dosing at levels that achieve very high maximum concentrations (Cmax) in vivo.

In some embodiments, the pharmaceutically active agent is cabazitaxel and the macromolecule has a lower maximal concentration (Cmax) of cabazitaxel in comparison to the direct administration of an equivalent dose of free cabazitaxel. In some embodiments, the macromolecule has a lower maximal concentration (Cmax) of cabazitaxel in comparison to the direct administration of an equivalent dose of free cabazitaxel when used in a method of treatment, for example, in the treatment of cancer, such as hormone-refractory prostate cancer, metastatic castration-resistant prostate cancer (mCRPC), or breast cancer. In some embodiments, administration of the macromolecule provides a maximal concentration (Cmax) of drug which is less than 90%, less than 50%, less than 40%, less than 30%, less than 20%, less than 10%, or less than 5% of the Cmax which results from direct administration of an equivalent dose of free cabazitaxel. In some embodiments, an amount of macromolecule is administered which is sufficient to provide a maximal concentration (Cmax) of unconjugated Cabazitaxel of less than 800, less than 500, less than 100, less than 50, less than 25, less than 15, less than ten, or less than five ng/mL.

As discussed above, a macromolecule according to the present disclosure has been shown to have sustained exposure when administered in vivo. In some embodiments, the pharmaceutically active agent is cabazitaxel and cabazitaxel released from the macromolecule has an increased terminal phase half-life (t1/2) in comparison to the direct administration of an equivalent dose of free cabazitaxel. The half-life of a drug is the time it takes for the blood plasma concentration of the drug to halve. It will be appreciated that an increased (i.e., longer) half-life may be desirable since it results in exposure to therapeutically effective concentrations of drug for a longer period of time. It also results in the need for less frequent dosing.

In some embodiments, the pharmaceutically active agent is cabazitaxel and cabazitaxel released from the macromolecule has an increased terminal phase half-life (t1/2) in comparison to the direct administration of an equivalent dose of free cabazitaxel when used in a method of treatment, for example, in the treatment of cancer, such as hormone-refractory prostate cancer, metastatic castration-resistant prostate cancer (mCRPC), or breast cancer. In some embodiments, administration of the macromolecule results in a pharmacokinetic profile for released cabazitaxel in which the terminal phase half-life (t1/2) is 1.5 times, at least 2 times, at least 3 times, at least 4 times, at least 5 times, or at least 10 times the half-life of cabazitaxel observed on administration of an equivalent dose of free cabazitaxel.

Free cabazitaxel is characterized by a triphasic PK model with an initial-phase half-life averaging 4 minutes, followed by an intermediate-phase half-life of 2 hours, and a prolonged terminal-phase half-life averaging 95 hours. In some embodiments, administration of the dendrimer provides a terminal phase half-life (t1/2) for unconjugated/released cabazitaxel of at least 12 hours, at least 24 hours, at least 30 hours, at least 40 hours, at least 48 hours, or at least 50 hours.

It will be appreciated that any one or more of improved therapeutic drug exposure (AUC), a lower maximal concentration (Cmax) of the drug, an increased half-life (t1/2), and reduced toxicity of the drug, may provide better clinical efficacy in comparison to the direct administration of the free drug. In some embodiments, administration of the macromolecule provides better efficacy of the drug, in comparison to the direct administration of an equivalent dose of the free drug. In some embodiments, the pharmaceutically active agent is cabazitaxel and administration of the macromolecule provides enhanced clinical efficacy in comparison to administration of an equivalent dose of free cabazitaxel. In some embodiments, the pharmaceutically active agent is cabazitaxel and the macromolecule provides an improved efficacy property selected from the group consisting of progression free survival, time to progression, objective response rate (PR+CR), overall response rate, overall survival and duration of response, in comparison to direct administration of an equivalent dose of free cabazitaxel.

Some embodiments will now be described with reference to the following Examples which illustrate some particular aspects and embodiments. However, it is to be understood that the particularity of the following description of some embodiments is not to supersede the generality of the preceding description of the embodiments.

### Abbreviations:

| | | | |
|---|---|---|---|
| Aba | Acetylbutyric acid | Gem | Gemcitabine |
| Ab | Antibody | Glu | Glutaric acid |
| Ac | Acetyl | HPLC | High Performance Liquid |
| | | | Chromatography |
| ACN | Acetonitrile | HSBA | Hydrazinosulfonyl |
| | | | benzoic acid |
| Av | Streptavadin | LCMS | Liquid chromatography |
| | | | mass spectrometry |
| BHAlysine | Benzhydrylamide lysine | MeOH | Methanol |
| Boc | benzyloxycarbonyl | MIDA | Methyliminodiacetic acid |
| Cp | Oxo-cyclopentane | PBS | Phosphate buffered saline |
| | carboxylic acid | | |
| DBCO | Dibenzenecyclooctyne | o-PDA | Ortho-phenylenedioxydi- |
| | | | acetic acid |
| DCC | Dicyclohexylcarbodiimide | PDT | 3,4- |
| | | | propylenedioxythiophene- |
| | | | 2,5-dicarboxylic acid |
| DCM | Dichloromethane | PEG | Polyethylene glycol |
| DGA | Diglycolic acid | PSSP | Dithiopropanoic acid |
| DIPEA | diisopropylethylamine | PTX | Paclitaxel |
| DMAP | dimethylaminopyridine | PyBop | Benzotriazol-1-yl-oxytri- |
| | | | pyrrolidinophosphonium |
| | | | hexafluorophosphate |
| DMF | Dimethylformamide | SB | Salbutamol |
| EtOAc | Ethyl acetate | SEC | Size exclusion |
| | | | chromatography |
| DTX | Docetaxel | SRB | Sulforhodamine B |
| EDC | 1-ethyl-3-(3-dimethyl- | TDA | 2,2'-thiodiacetic acid |
| | aminopropyl)carbo- | | |
| | diimide | | |
| ESI | Electrospray ionisation | TFA | Trifluoroacetic acid |

### EXAMPLES

The dendrimers represented in the examples below include reference to the core and the building units in the outermost generation of the dendrimer. The 1st to subsurface generations are not depicted. The dendrimer BHALys[Lys]₃₂ is representative of a 5 generation dendrimer having the formula BHALys[Lys]₂[Lys]₄[Lys]₈[Lys]₁₆[Lys]₃₂, the 64 surface amino groups being available to bind to terminal groups.

Preparation of the dendrimer scaffolds BHALys[Lys]₃₂[α-NH₂·TFA]₃₂[ε-PEG₅₇₀]₃₂, BHALys[Lys]₃₂[α-NH₂·TFA]₃₂[ε-PEG₁₁₀₀]₃₂, BHALys[Lys]₃₂[α-NH₂·TFA]₃₂[ε-t-PEG₂₃₀₀]₃₂BHALys[Lys]₃₂[α-4-HSBA]₃₂[ε-PEG₁₁₀₀]₃₂, BHALys[Lys]₃₂[α-GILGVP-NH₂·TFA]₃₂[ε-PEG₁₁₀₀]₃₂, and BHALys[Lys]₃₂[α-GILGVP-NH₂·TFA]₃₂[ε-t-PEG₂₃₀₀]₃₂ can be found in Kaminskas et al., J Control. Release (2011) doi 10.1016/j.jconrel.2011.02.005. Preparation of the dendrimer scaffolds 4-azidobenzamide-PEG₁₂-NEOEOEN[Su(NPN)₂][Lys]₁₆[NH₂·TFA]₃₂ can be found WO08/017122.

### General Procedures

### General Procedure A. Installation of Linkers to Drugs A

To a magnetically stirred solution of carboxylic acid linker (0.2-0.5 mmol) in solvent DMF or acetonitrile (1-5 mL) at 0° C. was added coupling agent either EDC or DCC (1.2 equivalents). The mixture was left to stir for 5 min., then a solution of solvent (1 mL) containing a mixture of drug (0.4-1 equivalents) and DMAP (0.4-1 equivalents) was added dropwise. The mixture was kept at 0° C. for 1 hour then allowed to warm to ambient temperature. The volatiles were then removed in vacuo and the residue purified by preparative HPLC (BEH 300 Waters XBridge C18, 5 μM, 30×150 mm, 40-80% ACN/water (5-40 min), no buffer) to yield the desired product.

### General Procedure B. Installation of Linkers to Drugs B.

To a magnetically stirred solution of drug (0.3-1.0 mmol) and anhydride (2 equivalents) in DMF (3-5 mL) was added DIPEA (3 equivalents). The mixture was stirred at ambient temperature overnight. The volatiles were then removed in vacuo and the residue purified by preparative HPLC (BEH 300 Waters XBridge C18, 5 μM, 30×150 mm, 40-70% ACN/water (5-40 min), no buffer, RT=34 min). The appropriate fractions were concentrated in vacuo providing the desired target.

General Procedure C. Loading Dendrimer with Drug-Linker.

To a magnetically stirred mixture of BHALys[Lys]₃₂[α-NH₂. TFA]₃₂[ε-PEG₁₁₀₀]₃₂ (0.5-1.0 μmol) and DIPEA (1.2 equivalents per amine) in DMF at room temperature was added linker-drug (1.2 equivalents per amine group) and PyBOP (1.2 equivalents per amine group). After 1.5 hours at room temperature the volatiles were removed and the residue purified by SEC (sephadex, LH20, MeOH). The appropriate fractions, as judged by HPLC, were combined and concentrated to provide the desired material.

### General Procedure D. Click Reaction

To a magnetically stirred solution dendrimer (0.5-1.0 mmol) in 1:1 H₂O/t-BuOH (approximately 0.5 mL) was added alkyne reagent (2 equivalents), sodium ascorbate solution (2 equivalents) and CuSO₄ solution (20 mol %). The solution was heated at 80° C. and monitored by HPLC. Additional charges of both sodium ascorbate and CuSO₄ were added as required to drive the reaction to completion. After the reaction was judged complete the reaction was concentrated in vacuo and then purified.

### Example 1

### (a) Preparation of 4-Aba-DTX:

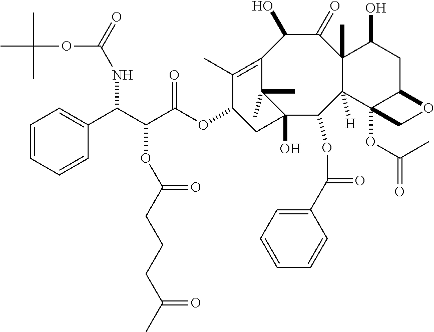

Prepared using Procedure A above, using DTX (200 mg, 0.25 mmol) and 4-acetylbutyric acid (42 mg, 0.32 mmol) as the linker. Preparative HPLC (RT=32 mins) provided 73 mg (32%) of product as a white solid. LCMS (C8, gradient: 40-90% ACN/H₂O (1-7 min), 90% ACN (7-9 min), 90-40% ACN (9-11 min), 40% ACN (11-15 min), 0.1% TFA) Rt (min)=7.60. ESI (+ve) observed [M+H]⁺=920. Calculated for C₄₉H₆₁NO₁₆=919.40 Da. ¹H NMR (300 MHz, CD₃OD) δ (ppm): 1.09 (s, 3H), 1.13 (s, 3H), 1.38 (s, 9H), 1.66 (s, 3H), 1.74-1.97 (m, 7H), 2.10 (s, 3H), 2.12-2.36 (m, 1H), 2.29-2.58 (m, 8H), 3.83 (d, J=6.9 Hz, 1H), 4.14-4.26 (m, 3H), 4.95-5.05 (m, 2H), 5.18-5.35 (m, 3H), 5.61 (d, J=7.2 Hz, 1H), 6.05 (m, 1H), 7.17-7.20 (m, 1H), 7.23-7.45 (m, 4H), 7.52-7.62 (m, 2H), 7.63-7.72 (m, 1H), 8.10 (d, J=7.2 Hz, 2H).

(b) Preparation of BHALys[Lys]₃₂[α-4-HSBA-4Aba-DTX]₃₂[ε-PEG₁₁₀₀]₃₂

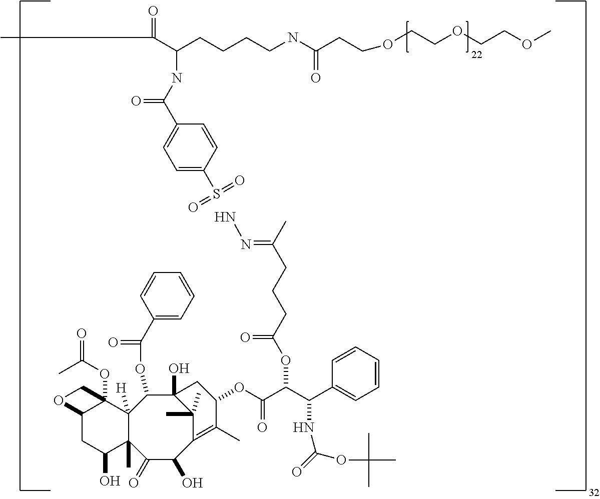

Prepared using Procedure C above. To a magnetically stirred solution of 4-Aba-DTX (15 mg, 16.3 μmol) in dry MeOH (1 mL) was added TFA (50 μL) and BHALys[Lys]₃₂[α-4-HSBA]₃₂[ε-PEG₁₁₀₀]₃₂ (20 mg, 0.43 μmol). The mixture was left to stir overnight at ambient temperature then added directly to a sephadex column (LH20, MeOH) for purification. The appropriate fractions, as judged by HPLC, were combined and concentrated to provide 25 mg (78%) of desired material as a white solid. HPLC (C8, gradient: 40-80% ACN/H₂O (1-7 min), 80% ACN (7-9 min), 80-40% ACN (9-11 min), 40% ACN (11-15 min), 10 mM ammonium formate) Rt (min)=6.77. ¹H NMR (300 MHz, CD₃OD) δ (ppm): 0.6-2.2 (m, 812H), 2.2-2.5 (m, 115H), 2.9-3.2 (m, 78H), 3.26 (s, 79H), 3.3-3.8 (m, 2824H), 5.1-5.3 (m, 31H), 5.5-5.6 (m, 10H), 5.9-6.1 (m, 9H), 6.9-8.2 (m, 329H). Theoretical molecular weight of conjugate: 78.6 kDa. ¹H NMR indicates 9 DTX/dendrimer. Actual molecular weight is approximately 56.4 kDa (13% DTX by weight).

### Example 2

### (a) Preparation of PSSP-DTX:

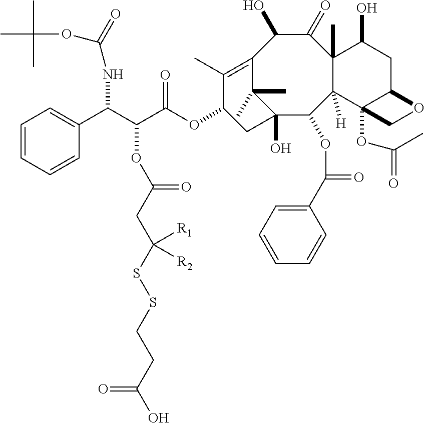

In this example (R₁═R₂═H) it could be envisioned that the rate of release of docetaxel could be increased or decreased by increasing or decreasing the degree of steric hindrance about the disulphide bond (Worrell N. R., Cumber A. J., Parnell G. D., Mirza A., Forrester J. A., Ross W. C. J.: Effect of linkage variation on pharmacokinetics of ricin-A-chainantibody conjugates in normal rats. Anti-Cancer Drug Design 1, 179, 1986). This could be achieved through the addition of substituents, amongst others a and or β to the disulphide bond. This type of tuning strategy is often used in prodrug design strategies and takes advantage of the well known Thorpe-Ingold or gem-dimethyl effect (The gem-Dimethyl Effect Revisited Steven M. Bachrach, J. Org. Chem. 2008, 73, 2466-2468).

Prepared using Procedure A above, using DTX (500 mg, 0.62 mmol) and 3,3′-dithiopropionic acid (130 mg, 0.62 mmol) as the linker. Preparative HPLC (RT=32 min) provided 179 mg (29%) of product as a white solid. LCMS (C8, gradient: 40-90% ACN/H₂O (1-7 min), 90% ACN (7-9 min), 90-40% ACN (9-11 min), 40% ACN (11-15 min), 0.1% TFA) Rf (min)=7.57. ESI (+ve) observed [M+H]⁺=1000. Calculated for C₄₉H₆₁NO₁₇S₂=999.34 Da. ¹H NMR (300 MHz, CD₃OD) δ (ppm): 1.13 (s, 3H), 1.17 (s, 3H), 1.43 (s, 9H), 1.70 (s, 3H), 1.72-1.99 (m, 6H), 2.13-2.32 (m, 1H), 2.37-2.55 (m, 4H), 2.66-2.76 (m, 2H), 2.76-3.02 (m, 6H), 3.87 (d, J=6.9 Hz, 1H), 4.18-4.31 (m, 3H), 5.00-5.06 (m, 3H), 5.24-5.42 (m, 3H), 5.64 (d, J=7.2 Hz, 1H), 6.10 (m, 1H), 7.23-7.33 (m, 1H), 7.36-7.48 (m, 4H), 7.53-7.65 (m, 2H), 7.66-7.76 (m, 1H), 8.13 (d, J=7.2 Hz, 2H).

(b) Preparation of BHALys[Lys]₃₂[α-PSSP-DTX]₃₂[ε-PEG₁₁₀₀]₃₂

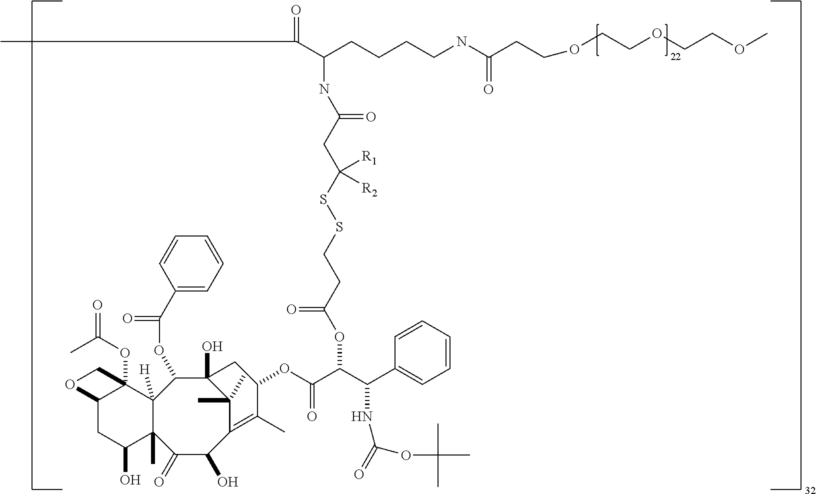

R₁═R₂═H

Prepared using Procedure C above, using BHALys[Lys]₃₂[α-NH₂·TFA]₃₂[ε-PEG₁₁₀₀]₃₂ (34 mg, 0.78 μmol) and PSSP-DTX (30 mg, 30 μmol). Purification by SEC provided 50 mg (89%) of desired material as a white solid. HPLC (C8, gradient: 40-80% ACN/H₂O (1-7 min), 80% ACN (7-9 min), 80-40% ACN (9-11 min), 40% ACN (11-15 min), 10 mM ammonium formate) Rf (min)=7.96 min. ¹H NMR (300 MHz, CD₃OD) δ (ppm): 0.7-2.0 (m, 1041H), 2.0-2.2 (m, 15H), 2.2-2.5 (m, 119H), 2.5-2.7 (m, 31H), 2.7-3.0 (m, 119H), 3.0-3.2 (m, 68H), 3.26 (s, 132H), 3.3-3.8 (m, 2806H), 3.9-4.3 (m, 76H), 5.1-5.3 (m, 55H), 5.5-5.6 (m, 17H), 5.9-6.1 (m, 17H), 7.1-8.1 (m, 243H). Theoretical molecular weight of conjugate: 74.9 kDa. ¹H NMR indicates 17 DTX/dendrimer. Actual molecular weight is approximately 56.1 kDa (24% DTX by weight).

### Example 3

### (a) Preparation of DGA-DTX:

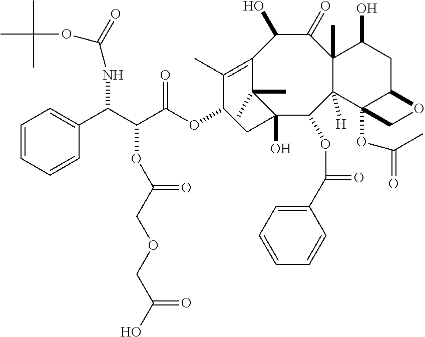

Prepared using Procedure B above, using DTX (300 mg, 371 μmol) and diglycolic anhydride (86 mg, 742 μmol) as the linker. Preparative HPLC (RT=34 min) provided 85 mg (25%) of DGA-DTX as a white solid. LCMS (C8, gradient: 40-90% ACN/H₂O (1-7 min), 90% ACN (7-9 min), 90-40% ACN (9-11 min), 40% ACN (11-15 min), 0.1% Formic acid) Rt (min)=5.90. ESI (+ve) observed [M+H]⁺=924.10. Calculated for C₄₇H₅₇NO₁₈=923.36 Da. ¹H NMR (300 MHz, CDCl₃) δ (ppm): 1.11 (s, 3H), 1.21 (s, 3H), 1.33 (s, 9H), 1.58-2.66 (m, 7H), 1.73 (s, 3H), 1.93 (s, 3H), 2.67-3.67 (br s, 5H), 3.73-3.97 (br s, 1H), 4.02-4.68 (m, 7H), 4.96 (d, J=8.4 Hz, 1H), 5.24 (s, 1H), 5.35-5.55 (m, 1H), 5.50 (s, 1H), 5.66 (d, J=6.7 Hz, 1H), 5.95-6.30 (m, 1H), 7.24-7.68 (m, 7H), 8.08 (d, J=6.9 Hz, 2H).

(b) Preparation of BHALys[Lys]₃₂[α-DGA-DTX]₃₂[ε-PEG₁₁₀₀]₃₂

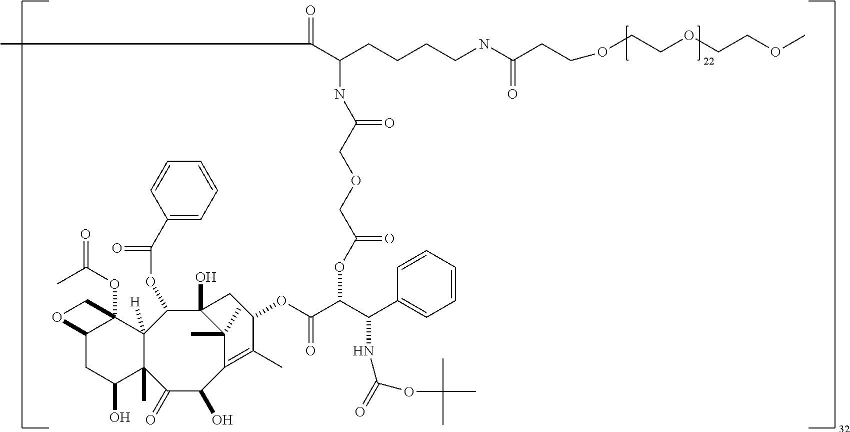

Prepared using Procedure C above, using BHALys[Lys]₃₂[α-NH₂. TFA]₃₂[ε-PEG₁₁₀₀]₃₂ (36 mg, 0.84 μmol) and DGA-DTX (30 mg, 33 μmol). Purification by SEC provided 45 mg (79%) of desired material as a white solid. HPLC (C8, gradient: 40-80% ACN/H₂O (1-7 min), 80% ACN (7-9 min), 80-40% ACN (9-11 min), 40% ACN (11-15 min), 10 mM ammonium formate) Rt (min)=7.69. ¹H NMR (300 MHz, CD₃OD) δ (ppm): 1.0-2.1 (m, 833H), 2.3-2.6 (m, 125H), 3.0-3.3 (m, 68H), 3.5-4.0 (m, 2803H), 4.0-4.7 (m, 214H), 5.0-5.1 (m, 23H), 5.3-5.5 (m, 54H), 5.6-5.8 (m, 19H), 6.0-6.3 (m, 18H), 7.2-7.8 (m, 203H), 8.1-8.2 (m, 46H). Theoretical molecular weight of conjugate: 72.4 kDa. ¹H NMR indicates 18 DTX/dendrimer. Actual molecular weight is approximately 55.7 kDa (26% DTX by weight).

### Example 4

### (a) Preparation of Cp-DTX:

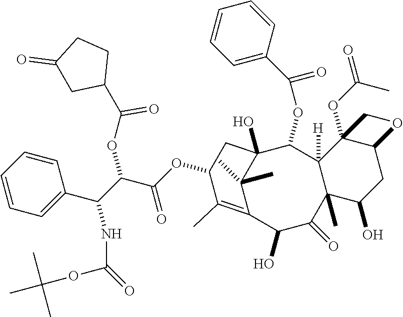

Prepared using Procedure A above, using DTX (500 mg, 619 μmol) and 3-oxo-1-cyclopentanecarboxylic acid (79 mg, 619 μmol) as the linker. Preparative HPLC (RT=33.5 min) provided Cp-DTX (401 mg, 71%) as a white solid. LCMS (C8, gradient: 40-90% ACN/H₂O (1-7 min), 90% ACN (7-9 min), 90-40% ACN (9-11 min), 40% ACN (11-15 min), 0.1% Formic acid) Rt (min)=6.61. ESI (+ve) observed [M+H]⁺=918.54. Calculated for C₄₉H₅₉NO₁₆=917.38 Da. ¹H NMR (300 MHz, CDCl₃) δ (ppm): 1.13 (s, 3H), 1.24 (s, 3H), 1.33 (s, 9H), 1.76 (s, 3H), 1.77-2.01 (m, 3H), 1.95 (s, 3H), 2.11-2.49 (m, 6H), 2.46 (s, 3H), 2.60 (ddd, J=16.2, 9.9 and 6.9 Hz, 1H), 3.10-3.24 (m, 1H), 3.94 (d, J=7.2 Hz, 1H), 4.20 (d, J=8.4 Hz, 1H), 4.27 (dd, J=11.1 and 6.6 Hz, 1H), 4.33 (d, J=8.4 Hz, 1H), 4.97 (d, J=7.8 Hz, 1H), 5.21 (s, 1H), 5.33 (d, J=9.9 Hz, 1H), 5.42 (d, J=2.7 Hz, 1H), 5.48-5.58 (br d, J=9 Hz, 1H), 5.69 (d, J=7.2 Hz, 1H), 6.27 (t, J=8.7 Hz, 1H), 7.25-7.45 (m, 5H), 7.47-7.53 (m, 2H), 7.57-7.64 (m, 1H), 8.09-8.14 (m, 2H).

### (b) Preparation of 4-HSBA-Cp-DTX:

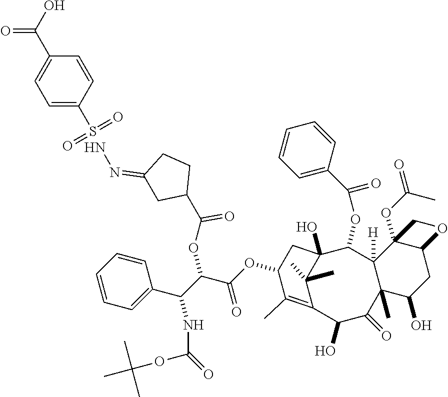

A solution of DTX-Cp (30 mg, 32.7 μmol) in TFA/MeOH (5% v/v, 1 mL) was added to 4-hydrazinosulfonylbenzoic acid (6 mg, 27.8 μmol). The mixture was left to react at 38° C. for 1.5 h after which the solvent was evaporated in vacuo. The white semi-solid obtained was used directly in the next step.

(c) Preparation of BHALys[Lys]₃₂[α-4-HSBA-Cp-DTX]₃₂[ε-PEG₁₁₀₀]₃₂

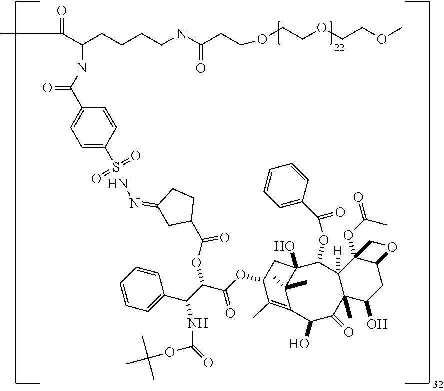

Method A: To a magnetically stirred solution of Cp-DTX (7.5 mg, 8.15 μmol) in dry MeOH (1 mL) was added TFA (50 μL). This solution was added to BHALys[Lys]₃₂[α-4-HSBA]₃₂[ε-PEG₁₁₀₀]₃₂ (10 mg, 0.215 μmol). The mixture was left to react overnight at ambient temperature then added directly to a sephadex column (LH20, MeOH) for purification. The appropriate fractions, as judged by HPLC, were combined, concentrated and freeze-dried from water to provide 18 mg (70%) of desired material as a white solid.

Method B: To 4-HSBA-Cp-DTX (31 mg, 27.8 μmol) and PyBOP (14.5 mg, 27.8 μmol) was added a solution of BHALys[Lys]₃₂[α-NH₂·TFA]₃₂[ε-PEG₁₁₀₀]₃₂ (31.5 mg, 0.7 μmol) and DIPEA (15 μL, 89.0 μmol) in DMF (1 mL). The resulting mixture was stirred overnight at ambient temperature after which the solvent was evaporated in vacuo. The remaining yellow oil was added to a sephadex column (LH20, MeOH) for purification. The appropriate fractions, as judged by HPLC, were combined, concentrated and freeze-dried from water to provide 34 mg (81% over two steps) of desired material as a white solid. HPLC (C8, gradient: 40-80% ACN/H₂O (1-7 min), 80% ACN (7-9 min), 80-40% ACN (9-11 min), 40% ACN (11-15 min), 10 mM ammonium formate) Rt (min)=7.65. ¹H NMR (300 MHz, CD₃OD) δ (ppm): 1.12 (s, 44H), 1.16 (s, 44H), 1.21-2.29 (m, 688H), 2.32-2.53 (m, 113H), 2.80-3.25 (m, 64H), 3.35 (s, 85H), 3.36-3.90 (m, 2815H), 4.17-4.28 (m, 77H), 4.45-4.65 (m, 50H), 4.97-5.04 (m, 23H), 5.22-5.44 (m, 40H), 5.63 (d, J=6.9 Hz, 16H), 6.00-6.20 (m, 15H), 7.2-8.25 (m, 308H). Theoretical molecular weight of conjugate: 78.8 kDa. ¹H NMR indicates 15 DTX/dendrimer in each case. Actual molecular weight is approximately 60.0 kDa (20% DTX by weight).

### Example 5

### (a) Preparation of Glu-DTX:

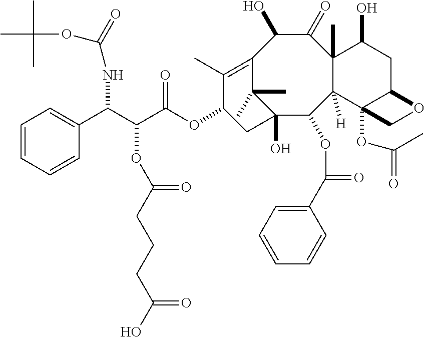

Prepared using Procedure B above, using DTX (300 mg, 371 μmol) and glutaric anhydride (85 mg, 742 μmol) in DMF (3.7 mL) as the linker. Preparative HPLC (Rt=33 min) provided 106 mg (31%) of Glu-DTX as a white solid. LCMS (C8, gradient: 40-90% ACN/H₂O (1-7 min), 90% ACN (7-9 min), 90-40% ACN (9-11 min), 40% ACN (11-15 min), 0.1% Formic acid) Rt (min)=6.12. ESI (+ve) observed [M+H]⁺=922.13. Calculated for C₄₈H₅₉NO₁₇=921.38 Da. ¹H NMR (300 MHz, CDCl₃) δ (ppm): 1.11 (s, 3H), 1.22 (s, 3H), 1.33 (s, 9H), 1.74 (s, 3H), 1.79-2.65 (m, 14H), 1.93 (s, 3H), 3.91 (d, J=6.5 Hz, 1H), 4.19 (d, J=8.4 Hz, 1H), 4.26 (dd, J=11.1 and 6.9 Hz, 1H), 4.31 (d, J=8.4 Hz, 1H), 4.96 (d, J=8.2 Hz, 1H), 5.23 (s, 1H), 5.38 (br s, 1H), 5.35-5.65 (br d, 1H), 5.67 (d, J=6.5 Hz, 1H), 6.10-6.30 (s, 1H), 7.26-7.34 (m, 3H), 7.34-7.43 (m, 2H), 7.46-7.55 (m, 2H), 7.57-7.65 (m, 1H), 8.10 (d, J=7.4 Hz, 2H).

(b) Preparation of BHALys[Lys]₃₂[α-Glu-DTX]₃₂[ε-PEG₁₁₀₀]₃₂

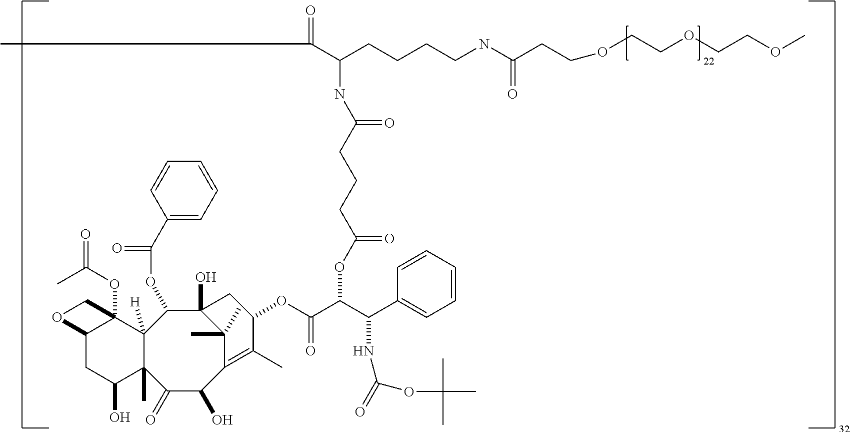

Prepared using Procedure C above, using BHALys[Lys]₃₂[α-NH₂·TFA]₃₂[ε-PEG₁₁₀₀]₃₂ (50 mg, 1.1 μmol) and Glu-DTX (39 mg, 42.3 μmol). Purification by sephadex column (LH20, MeOH) provided 49.5 mg (78%) of desired material as a white solid. HPLC (C8, gradient: 40-80% ACN/H₂O (1-7 min), 80% ACN (7-9 min), 80-40% ACN (9-11 min), 40% ACN (11-15 min), 10 mM ammonium formate) Rt (min)=7.78. ¹H NMR (300 MHz, CD₃OD) δ (ppm): 1.00-2.10 (m, 1037H), 2.10-2.74 (m, 296H), 3.05-3.27 (br s, 88H), 3.35 (s, 96H), 3.36-3.78 (m, 2800H), 3.80-3.93 (m, 42H), 4.01-4.47 (m, 125H), 4.47-4.60 (br s, 23H), 4.92-5.08 (br s, 30H), 5.18-5.45 (m, 70H), 5.54-5.74 (br s, 22H), 6.00-6.23 (br s, 20H), 7.15-7.75 (m, 414H), 8.05-8.20 (br d, J=6.4 Hz, 49H). Theoretical molecular weight of conjugate: 72.6 kDa. ¹H NMR indicates 20 DTX/dendrimer. Actual molecular weight is approximately 57.5 kDa (28% DTX by weight).

### Example 6

### (a) Preparation of MIDA-DTX:

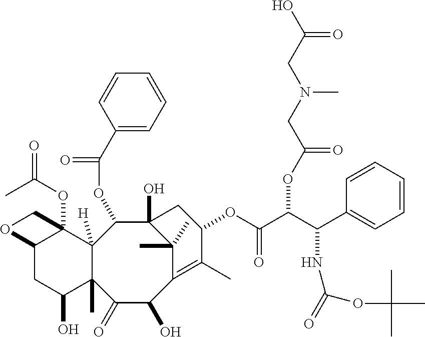

Prepared using Procedure A above, using DTX (100 mg, 124 μmol) and methyliminodiacetic acid (91 mg, 620 μmol) as the linker. Preparative HPLC (RT=22.5 min) provided 29 mg (25%) of product as a white solid. LCMS (C8, gradient: 40-90% ACN/H₂O (1-7 min), 90% ACN (7-9 min), 90-40% ACN (9-11 min), 40% ACN (11-15 min), 0.1% Formic acid) Rt (min)=4.62. ESI (+ve) observed [M+H]⁺=937.34. Calculated for C₄₈H₆₀N₂O₁₇=936.39 Da. ¹H NMR (300 MHz, CD₃OD) δ (ppm): 1.13 (s, 3H), 1.17 (s, 3H), 1.40 (s, 9H), 1.70 (s, 3H), 1.84 (ddd, J=14.1, 11.4 and 1.8 Hz, 1H), 1.93 (s, 3H), 2.04 (dd, J=15.0 and 8.7 Hz, 1H), 2.30 (dd, J=15.0 and 8.7 Hz, 1H), 2.43 (s, 3H), 2.46 (ddd, J=14.1, 9.5 and 6.6 Hz, 1H), 2.61 (s, 3H), 3.49 (s, 2H), 3.81-3.94 (m, 3H), 4.21 (s, 2H), 4.24 (dd, J=11.4 and 6.6 Hz, 1H), 5.01 (dd, J=9.5 and 1.8 Hz, 1H), 5.29 (s, 1H), 5.43 (s, 2H), 5.65 (d, J=7.2 Hz, 1H), 6.16 (t, J=8.7 Hz, 1H), 7.21-7.34 (m, 1H), 7.35-7.50 (m, 4H), 7.51-7.79 (m, 3H), 8.13 (d, J=7.2 Hz, 2H).

(b) Preparation of BHALys[Lys]₃₂[α-MIDA-DTX]₃₂[ε-PEG₁₁₀₀]₃₂

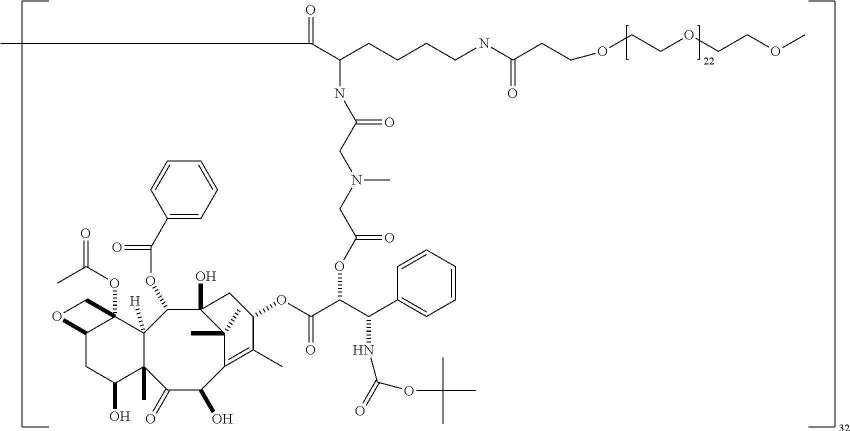

Prepared using Procedure C above, using BHALys[Lys]₃₂[α-NH₂. TFA]₃₂[ε-PEG₁₁₀₀]₃₂ (31.5 mg, 0.7 μmol) and MIDA-DTX (26 mg, 27.8 μmol). Purification by SEC provided 41.6 mg (93%) of the desired product as a white solid. HPLC (C8, gradient: 40-80% ACN/H₂O (1-7 min), 80% ACN (7-9 min), 80-40% ACN (9-11 min), 40% ACN (11-15 min), 10 mM ammonium formate) Rt (min)=7.78. ¹H NMR (300 MHz, CD₃OD) δ (ppm): 1.00-2.10 (m, 1186H), 2.12-2.68 (m, 283H), 3.06-3.27 (m, 77H), 3.35 (s, 101H), 3.36-3.96 (m, 2842H), 4.07-4.61 (m, 143H), 4.93-5.10 (br s, 31H), 5.19-5.48 (m, 77H), 5.55-5.75 (m, 27H), 5.97-6.29 (m, 27H), 7.10-7.84 (m, 258H), 8.03-8.23 (m, 60H). Theoretical molecular weight of conjugate: 73.1 kDa. 1H NMR indicates 27 DTX/dendrimer. Actual molecular weight is approximately 64.2 kDa (34% DTX by weight).

### Example 7

(a) Preparation of o-PDA-DTX:

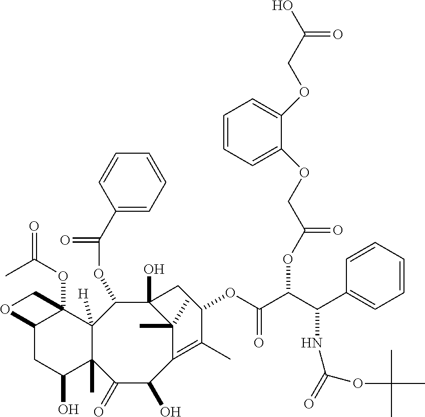

Prepared using Procedure A above, using DTX (300 mg, 0.37 mmol) and o-phenylenedioxydiacetic acid (419 mg, 1.85 mmol) as the linker. Preparative HPLC (RT=26 min) provided 21 mg (11%) of product as a white solid. LCMS (C8, gradient: 40-90% ACN/H₂O (1-7 min), 90% ACN (7-9 min), 90-40% ACN (9-11 min), 40% ACN (11-15 min), 0.1% Formic acid) Rt (min)=7.27. ESI (+ve) observed [M+H]⁺=1016.29. Calculated for C₅₃H₆₁NO₁₉=1015.38 Da. ¹H NMR (300 MHz, CD₃OD) δ (ppm): 1.13 (s, 3H), 1.17 (s, 3H), 1.40 (s, 9H), 1.69 (s, 3H), 1.82 (ddd, J=13.5, 11.4 and 2.1 Hz, 1H), 1.89 (s, 3H), 1.94-2.07 (m, 1H), 2.00-2.33 (m, 1H), 2.40 (s, 3H), 2.45 (ddd, J=15.9, 9.6 and 6.6 Hz, 1H), 3.87 (d, J=6.9 Hz, 1H), 4.18-4.27 (m, 3H), 4.68 (s, 2H), 4.87 (d, J=6.0 Hz, 1H), 5.00 (d, J=9.3 Hz, 1H), 5.27 (s, 1H), 5.36-5.43 (m, 2H), 5.64 (d, J=6.9 Hz, 1H), 6.13 (t, J=9.0 Hz, 1H), 6.86-6.98 (m, 4H), 7.23-7.32 (m, 1H), 7.35-7.43 (m, 4H), 7.52-7.60 (m, 2H), 7.62-7.70 (m, 1H), 8.07-8.15 (m, 2H).

(b) Preparation of BHALys[Lys]₃₂[α-o-PDA-DTX]₃₂[ε-PEG₁₁₀₀]₃₂

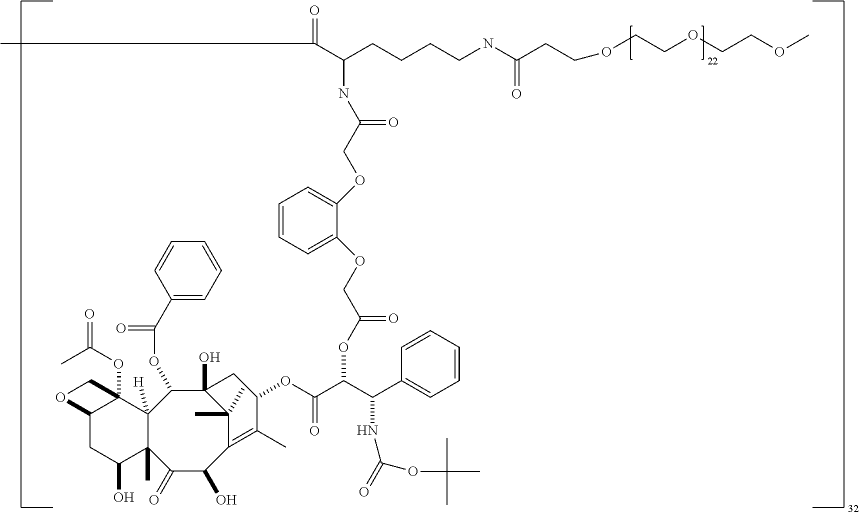

Prepared using Procedure C above, using BHALys[Lys]₃₂[α-NH₂. TFA]₃₂[ε-PEG₁₁₀₀]₃₂ (22.5 mg, 0.5 μmol) and o-PDA-DTX (21 mg, 20.7 μmol). Purification by SEC (sephadex, LH20, MeOH) provided 30 mg (95%) of the desired product as a slightly beige semi-solid. HPLC (C8, gradient: 40-80% ACN/H₂O (1-7 min), 80% ACN (7-9 min), 80-40% ACN (9-11 min), 40% ACN (11-15 min), 10 mM ammonium formate) Rt (min)=9.80. ¹H NMR (300 MHz, CD₃OD) δ (ppm): 0.95-2.12 (m, 1058H), 2.12-2.66 (m, 205H), 2.89-3.29 (m, 125H), 3.35 (s, 85H), 3.36-3.93 (m, 2822H), 3.98-4.75 (m, 212H), 4.83-5.08 (m, 89H), 5.18-5.34 (m, 17H), 5.34-5.54 (m, 38H), 5.54-5.79 (m, 22H), 6.01-6.26 (m, 22H), 6.68-7.13 (m, 98H), 7.13-7.78 (m, 214H), 8.02-8.22 (m, 50H). Theoretical molecular weight of conjugate: 75.6 kDa. ¹H NMR indicates 22 DTX/dendrimer. Actual molecular weight is approximately 63.2 kDa (28% DTX by weight).

### Example 8

### (a) Preparation of TDA-DTX Via Procedure A:

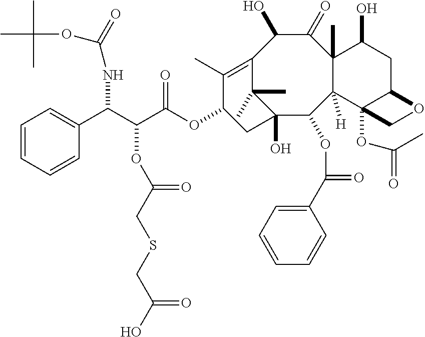

Prepared using Procedure A above, using DTX (500 mg, 0.62 mmol) and 2,2′-thiodiacetic acid (370 mg, 2.5 mmol) as the linker. Preparative HPLC (RT=33 min) provided 240 mg (41%) of product as a white solid. LCMS (C8, gradient: 40-90% ACN/H₂O (1-7 min), 90% ACN (7-9 min), 90-40% ACN (9-11 min), 40% ACN (11-15 min), 0.1% TFA) Rt (min)=10.60. ESI (+ve) observed [M+H]⁺=940. Calculated for C₄₇H₅₇NO₁₇S=939.33 Da. ¹H NMR (300 MHz, CD₃OD) δ (ppm): 1.15 (s, 3H), 1.19 (s, 3H), 1.43 (s, 9H), 1.72 (s, 3H), 1.78-2.05 (m, 2H), 1.93 (s, 3H), 2.16-2.57 (m, 2H), 2.43 (s, 3H), 3.36-3.63 (m, 2H), 3.89 (d, J=6.9 Hz, 1H), 4.18-4.34 (m, 3H), 5.03 (d, J=9.0 Hz, 2H), 5.28-5.44 (m, 3H), 5.66 (d, J=7.2 Hz, 1H), 6.11 (m, 1H), 7.24-7.35 (m, 1H), 7.38-7.50 (m, 4H), 7.52-7.65 (m, 2H), 7.66-7.76 (m, 1H), 8.14 (d, J=7.2 Hz, 2H).

### (b) Preparation of TDA-DTX Via Procedure B:

Prepared using Procedure B above, using DTX (400 mg, 0.50 mmol) and thiodiacetic anhydride (66 mg, 0.50 mmol) as the linker. The mixture was stirred at room temperature overnight then solvent was removed under reduced pressure to give a crude residue. The residue was re-dissolved in EtOAc (250 mL) and was washed with PBS buffer (adjusted to pH 4.0). The separated organic layer was dried over MgSO₄ and concentrated under reduced pressure to give 445 mg (95%) of the desired product as a white solid. LCMS (Waters XBridge C8 column (3.0×100 mm), 3.5 micron, 214, 243 nm, 0.4 mL/min, gradient: 40-90% ACN/H₂O (1-7 min), 90% ACN (7-9 min), 90-40% ACN (9-11 min), 40% ACN 11-15 min), 0.1% TFA) Rt (min)=10.60. ESI (+ve) observed [M+H]⁺=940. Calculated for C₄₇H₅₇NO₁₇S=939.33 Da.

(c) Preparation of BHALys[Lys]₃₂[α-TDA-DTX]₃₂[ε-PEG₁₁₀₀]₃₂

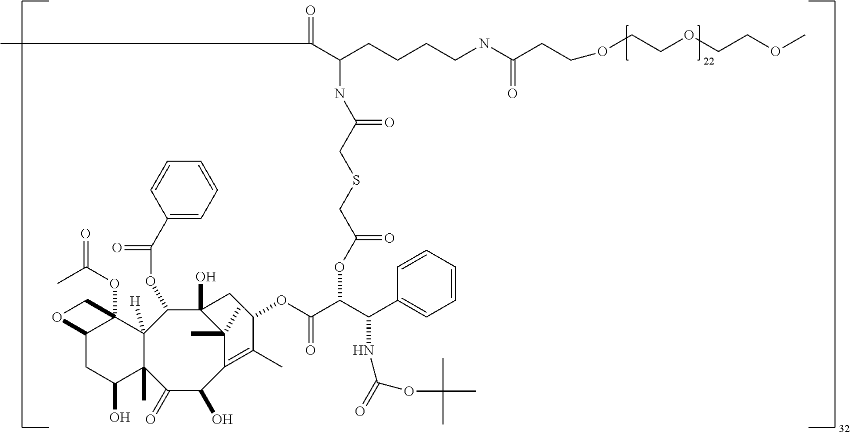

Prepared using Procedure C above, using BHALys[Lys]₃₂[α-NH₂. TFA]₃₂[ε-PEG₁₁₀₀]₃₂ (46 mg, 1.08 μmol) and TDA-DTX (44 mg, 47 μmol). Purification by SEC (sephadex, LH20, MeOH) provided 65 mg (87%) of desired material as a white solid. HPLC (C8, gradient: 40-80% ACN/H₂O (1-7 min), 80% ACN (7-9 min), 80-40% ACN (9-11 min), 40% ACN (11-15 min), 10 mM ammonium formate) Rt (min)=9.68. ¹H NMR (300 MHz, CD₃OD) δ (ppm): 0.78-2.02 (m, 809H), 2.27-2.58 (m, 114H), 3.03-3.24 (m, 43H), 3.34 (s, 73H), 3.37-3.96 (m, 2800H), 4.01-4.39 (m, 27H), 5.20-5.48 (m, 75H), 5.54-5.74 (m, 23H), 5.98-6.25 (m, 20H), 7.12-7.84 (m, 202H), 8.01-8.22 (m, 46H). Theoretical molecular weight of conjugate: 68.9 kDa. ¹H NMR indicates 23 DTX/dendrimer. Actual molecular weight is approximately 60.6 kDa (31% DTX by weight). Particle sizing using Dynamic Light Scattering shows a range of concentration dependent averages of 8.9-10.1 nm.

### Example 9

### (a) Preparation of PDT-DTX:

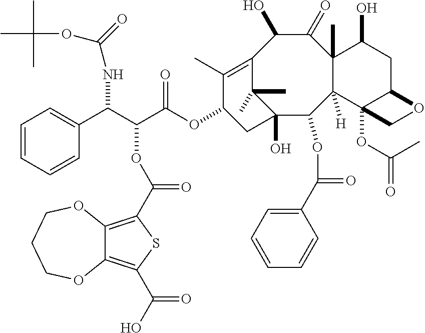

Prepared using Procedure A above, using DTX (250 mg, 0.31 mmol) and 3,4-propylenedioxythiophene-2,5-dicarboxylic acid (PDT, 75 mg, 0.31 mmol) as the linker. Purification by preparative HPLC (RT=28 min) provided 30 mg (9%) of product as a white solid. LCMS (C8, gradient: 40-90% ACN/H₂O (1-7 min), 90% ACN (7-9 min), 90-40% ACN (9-11 min), 40% ACN (11-15 min), 0.1% TFA) Rt (min)=7.24. ESI (+ve) observed [M+H]⁺=1034. Calculated for C₅₂H₅₉NO₁₉S=1033.34 Da. ¹H NMR (300 MHz, CD₃OD) δ (ppm): 1.14 (s, 3H), 1.18 (s, 3H), 1.45 (s, 9H), 1.71 (s, 3H), 1.78-1.91 (m, 2H), 1.94 (s, 3H), 2.09-2.27 (m, 1H), 2.29-2.58 (m, 3H), 2.41 (s, 3H), 3.88 (d, J=6.9 Hz, 1H), 4.20-4.30 (m, 3H), 4.31-4.43 (m, 4H), 4.94-5.16 (m, 1H), 5.30 (s, 1H), 5.36-5.42 (m, 2H), 5.65 (d, J=6.9 Hz, 1H), 6.02-6.22 (m, 1H), 7.23-7.34 (m, 1H), 7.36-7.53 (m, 4H), 7.56-7.65 (m, 2H), 7.66-7.77 (m, 1H), 8.11 (d, J=7.2 Hz, 2H).

(b) Preparation of BHALys[Lys]₃₂[α-PDT-DTX]₃₂[ε-PEG₁₁₀₀]₃₂

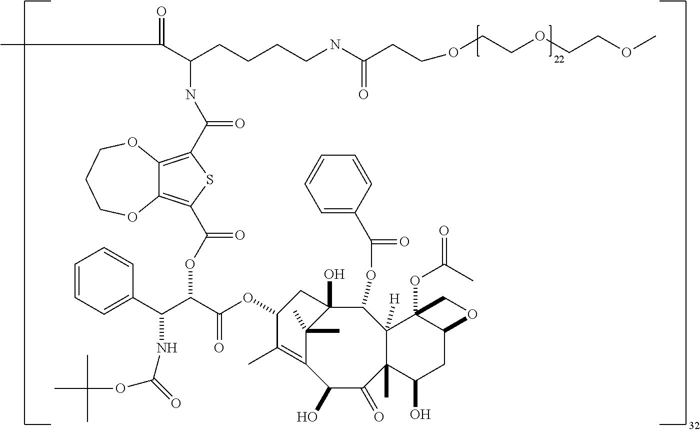

Prepared using Procedure C above, using BHALys[Lys]₃₂[α-NH₂. TFA]₃₂[ε-PEG₁₁₀₀]₃₂ (29 mg, 0.67 μmol) and PDT-DTX (30 mg, 29 μmol). Purification by SEC (sephadex, LH20, MeOH) provided 42 mg (88%) of desired material as a white solid. HPLC (C8, gradient: 40-80% ACN/H₂O (1-7 min), 80% ACN (7-9 min), 80-40% ACN (9-11 min), 40% ACN (11-15 min), 10 mM ammonium formate) Rt (min)=9.03. ¹H NMR (300 MHz, CD₃OD) δ (ppm): 0.76-2.10 (m, 974H), 2.23-2.66 (m, 210H), 3.08-3.30 (m, 74H), 3.40-3.98 (m, 2804H), 4.02-4.76 (m, 249H), 4.96-5.12 (m, 33H), 5.22-5.34 (m, 25H), 5.36-5.52 (m, 47H), 5.56-5.80 (m, 27H), 5.88-6.30 (m, 24H), 7.08-7.94 (m, 213H), 7.99-8.31 (m, 50H). Theoretical molecular weight of conjugate: 71.9 kDa. ¹H NMR indicates 26 DTX/dendrimer. Actual molecular weight is approximately 66.3 kDa (32% DTX by weight).

### Example 10

### (a) Preparation of PEG₂-DTX:

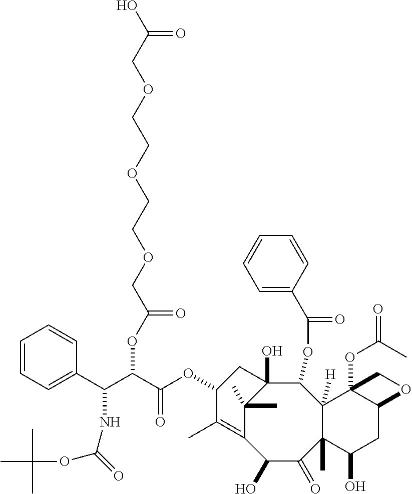

Prepared using Procedure A above, using DTX (200 mg, 0.25 mmol) and 3,6,9-trioxaundecanedioic acid (220 mg, 1.0 mmol). Preparative HPLC (RT=30.5 min) provided 70 mg (28%) of product as a white solid. LCMS (C8, gradient: 40-90% ACN/H₂O (1-7 min), 90% ACN (7-9 min), 90-40% ACN (9-11 min), 40% ACN (11-15 min), 0.1% Formic acid) Rt (min)=6.48. ESI (+ve) observed [M+H]⁺=1012.15. Calculated for C₅₁H₆₅NO₂₀=1011.41 Da. ¹H NMR (300 MHz, CD₃OD) δ (ppm): 1.13 (s, 3H), 1.17 (s, 3H), 1.40 (s, 9H), 1.70 (s, 3H), 1.83 (ddd, J=13.8, 11.1 and 2.1 Hz, 1H), 1.93 (s, 3H), 1.92-2.12 (m, 1H), 2.17-2.38 (m, 1H), 2.42 (s, 3H), 2.46 (ddd, J=14.7, 9.9 and 6.6 Hz, 1H), 3.56-3.82 (m, 8H), 3.88 (d, J=7.0 Hz, 1H), 4.06 (s, 2H), 4.16-4.39 (m, 5H), 5.01 (d, J=9.3 Hz, 1H), 5.29 (s, 1H), 5.38 (s, 2H), 5.65 (d, J=7.0 Hz, 1H), 6.13 (t, J=8.4 Hz, 1H), 7.22-7.33 (m, 1H), 7.35-7.47 (m, 4H), 7.51-7.62 (m, 2H), 7.62-7.72 (m, 1H), 8.13 (d, J=7.2 Hz, 2H).

(b) Preparation of BHALys[Lys]₃₂[α-PEG₂-DTX]₃₂[ε-PEG₁₁₀₀]₃₂

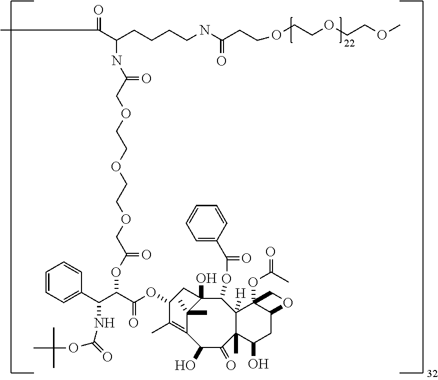

Prepared using Procedure C above, using BHALys[Lys]₃₂[α-NH₂. TFA]₃₂[ε-PEG₁₁₀₀]₃₂ (55.8 mg, 1.24 μmol) and PEG₂-DTX (50 mg, 49.5 μmol). Purification by SEC (sephadex, LH20, MeOH) provided 79 mg (>90%) of the desired product as a white solid. HPLC (C8, gradient: 40-80% ACN/H₂O (1-7 min), 80% ACN (7-9 min), 80-40% ACN (9-11 min), 40% ACN (11-15 min), 10 mM ammonium formate) Rf (min)=8.65. ¹H NMR (300 MHz, CD₃OD) δ (ppm): 0.91-2.14 (m, 968H), 2.14-2.64 (m, 185H), 2.88-3.29 (m, 109H), 3.35 (s, 89H), 3.36-3.95 (m, 3016H), 3.95-4.65 (m, 251H), 5.00 (br s, 32H), 5.20-5.49 (m, 72H), 5.55-5.75 (m, 25H), 6.13 (br s, 25H), 7.12-7.81 (m, 213H), 8.13 (d, J=7.2 Hz, 50H). Theoretical molecular weight of conjugate: 75.5 kDa. ¹H NMR indicates 24 DTX/dendrimer. Actual molecular weight is approximately 63.2 kDa (31% DTX by weight).

### Example 11

Preparation of BHALys[Lys]₃₂[α-Lys(α-Ac)(ε-DGA-DTX)]₃₂[ε-Lys(PEG 570)₂]32
(a) Preparation of HO-Lys (NH₂·TFA)₂

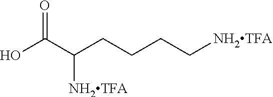

To a magnetically stirred suspension of L-lysine (500 mg, 3.42 mmol) in CH₂Cl₂ (21 mL) was added a solution of TFA in CH₂Cl₂ (21 mL, 1:1 v/v). The mixture was stirred at ambient temperature for 4 h, and then concentrated in vacuo. The residue was dissolved in water (30 mL) and concentrated in vacuo. This procedure was repeated once more. The remaining oil was then freeze-dried from water, providing 1.33 g of the desired product as a yellowish oil that was used directly in the next step.

(b) Preparation of HO-Lys(PEG₅₇₀)₂

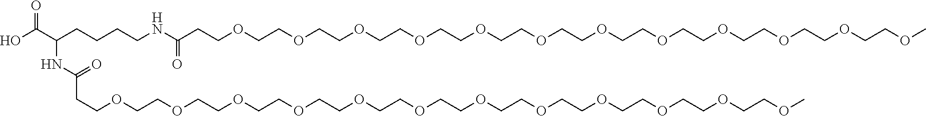 [SMILES: COCCOCCOCCOCCOCCOCCOCCOCCOCCOCCOCCOCCC(=O)NCCCCC(NC(=O)CCOCCOCCOCCOCCOCCOCCOCCOCCOCCOCCOCCOC)C(=O)O]

To a magnetically stirred solution of PEG₅₇₀-NHS (1.06 g, 1.55 mmol) in DMF (5 mL) was added DIPEA (806 μL, 4.64 mmol), followed by a solution of HO-Lys (NH₂·TFA)₂ (300 mg) in DMF (4 mL). The resulting mixture was stirred at ambient temperature overnight. The volatiles were then removed in vacuo and the residue purified by preparative HPLC (BEH 300 Waters XBridge C18, 5 μM, 30×150 mm, gradient: 5% ACN/H₂O (1-5 min), 5-60% ACN (5-35 min), 60-80% ACN (35-40 min), 80% ACN (40-45 min), 80-5% ACN (45-50 min), 5% ACN (50-60 min), no buffer, Rt=29.3 min). The appropriate fractions were concentrated in vacuo and freeze-dried in water, providing 481 mg (48% over two steps) of the desired product as a white semi-solid. HPLC (C18, gradient: 5-60% ACN/H₂O (1-10 min), 60% ACN (10-11 min), 60-5% ACN (11-13 min), 5% ACN (13-15 min), 10 mM ammonium formate) Rt (min)=8.68. ¹H NMR (300 MHz, CD₃OD) δ (ppm): 1.33-1.62 (m, 4H), 1.62-1.95 (m, 2H), 2.43 (t, J=6.2 Hz, 2H), 2.52 (dt, J=6.2 and 3.6 Hz, 2H), 3.16-3.24 (m, 2H), 3.36 (s, 6H), 3.36-3.90 (m, 95H), 4.39 (dd, J=8.7 and 5.1 Hz, 1H).

(c) Preparation of BHALys[Lys]₁₆[Lys(α-Boc)(ε-NH₂]₃₂

To a magnetically stirred suspension of BHALys[Lys]₁₆[Lys(α-Boc) (ε-Fmoc)]₃₂ (500 mg, 26.9 μmol) in DMF (3.4 mL) was added piperidine (849 μL, 20% v/v in DMF). The mixture was stirred at ambient temperature overnight, then poured into diethyl ether (65 mL). The white precipitate that formed was filtered off and washed with diethyl ether (100 mL). The filter cake was transferred to a vial and air dried for 3 days, providing 281 mg (91%) product as a white solid. ¹H NMR (300 MHz, CD₃OD) δ (ppm): 1.00-2.10 (m, 680H), 2.65-2.88 (br s, 48H), 2.91-2.98 (m, 11H), 2.99-3.28 (m, 78H), 3.81-4.21 (m, 33H), 4.21-4.55 (m, 32H), 6.21 (s, 1H), 7.20-7.41 (m, 10H).

(d) Preparation of BHALys[Lys]₃₂[α-Boc]₃₂[ε-Lys(PEG₅₇₀)₂]₃₂

To a magnetically stirred solution of BHALys[Lys]₁₆[Lys(α-Boc) (ε-NH₂)]₃₂ (49 mg, 4.33 μmol) in DMF and DMSO (3 mL, 5:1 v/v) was added DIPEA (96 μL, 554.2 μmol). The resulting solution was added to a solution of HO-Lys(PEG₅₇₀)₂ (223 mg, 173.3 μmol) and PyBOP (90 mg, 173.3 μmol) in DMF (5.5 mL). The mixture was stirred at ambient temperature overnight. The volatiles were then removed in vacuo and the residue purified by ultrafiltration (Pall Minimate™ Tangential Flow Filtration Capsules, Omega™ 10K Membrane, water). The remaining aqueous solution was freeze-dried, providing 120 mg (53%) of the desired product as a yellowish oil. ¹H NMR (300 MHz, CD₃OD) δ (ppm): 1.18-1.98 (m, 863H), 2.38-2.63 (m, 123H), 3.04-3.30 (m, 194H), 3.36 (s, 172H), 3.38-3.91 (m, 2816H), 3.93-4.18 (br s, 35H), 4.18-4.47 (m, 63H), 4.47-4.60 (m, 12H), 6.18 (s, 1H), 7.19-7.43 (m, 10H).

(e) Preparation of BHALys[Lys]₃₂[α-NH₂·TFA]₃₂[ε-Lys(PEG₅₇₀)₂]₃₂

To a magnetically stirred solution of BHALys[Lys]₃₂[α-Boc]₃₂[ε-Lys(PEG₅₇₀)₂]₃₂ (120 mg, 2.3 μmol) in CH₂Cl₂ (2 mL) was added a solution of TFA in CH₂Cl₂ (2 mL, 1:1 v/v). The mixture was stirred at ambient temperature for 3.5 h, after which the solvents were evaporated in vacuo. The remaining oil was dissolved in water (5 mL) and the resulting solution concentrated in vacuo. This procedure was repeated one more time and the oil that remained was taken up in water and purified by SEC (PD-10 desalting columns, GE Healthcare, 17-0851-01, sephadex G-25 medium). The collected fractions were combined and freeze-dried from water to provide 93 mg (77%) of desired material as a yellowish oil. ¹H NMR (300 MHz, CD₃OD) δ (ppm): 1.18-2.01 (m, 556H), 2.38-2.65 (m, 118H), 3.02-3.30 (m, 181H), 3.36 (s, 178H), 3.38-3.94 (m, 2816H), 4.09-4.55 (m, 63H), 6.13-6.22 (m, 1H), 7.19-7.45 (m, 10H).

(f) Preparation of BHALys[Lys]₃₂[α-Lys(α-Ac)(ε-Boc)]₃₂[ε-Lys(PEG 570)₂]₃₂

To a solution of BHALys[Lys]₃₂[α-NH₂. TFA]₃₂[ε-Lys(PEG₅₇₀)₂]₃₂ (93 mg, 1.8 μmol) in DMF (3.6 mL) was added DIPEA (40 μL, 230.4 μmol). The resulting solution was added to solid HO-Lys(α-Ac) (ε-Boc) (21 mg, 72 μmol) and PyBOP (37 mg, 72 μmol) contained in a second flask. The mixture was stirred at ambient temperature overnight. The volatiles were then removed in vacuo and the residue purified by SEC (sephadex, LH20, MeOH). The appropriate fractions, as judged by HPLC were combined and concentrated. The yellowish oil thus obtained was freeze dried from water to give 97 mg (94%) of the desired product as a slightly yellowish semi-solid. ¹H NMR (300 MHz, CD₃OD) δ (ppm): 1.10-2.15 (m, 1139H), 2.36-2.63 (m, 120H), 2.93-3.30 (m, 251H), 3.36 (s, 195H), 3.37-3.91 (m, 2816H), 4.16-4.51 (br s, 122H), 6.15-6.21 (m, 1H), 7.18-7.43 (m, 10H).

(g) Preparation of BHALys[Lys]₃₂[α-Lys(α-Ac)(ε-NH₂·TFA)]₃₂[ε-Lys(PEG 570)₂]₃₂

To a magnetically stirred solution of BHALys[Lys]₃₂[α-Lys(α-Ac)(ε-Boc)]₃₂[ε-Lys(PEG₅₇₀)₂]₃₂ (97 mg, 1.69 μmol) in CH₂Cl₂ (1 mL) was added a solution of TFA in CH₂Cl₂ (2 mL, 1:1 v/v). The mixture was stirred at ambient temperature overnight, and then the solvents were evaporated in vacuo. The remaining oil was dissolved in water (4 mL) and the resulting solution concentrated in vacuo. This procedure was repeated one more time and the oil that remained was taken up in water and purified by SEC (PD-10 desalting columns, GE Healthcare, 17-0851-01, sephadex G-25 medium). The collected fractions were combined and freeze-dried from water to provide 104 mg (>90%) of the desired material as a yellowish oil. ¹H NMR (300 MHZ, CD₃OD) δ (ppm): 1.13-2.20 (m, 843H), 2.37-2.65 (m, 122H), 2.89-3.06 (m, 70H), 3.06-3.30 (m, 180H), 3.36 (s, 182H), 3.39-3.92 (m, 2816H), 4.08-4.47 (br s, 126H), 6.13-6.20 (m, 1H), 7.20-7.45 (m, 10H).

(h) Preparation of BHALys[Lys]₃₂[α-Lys(α-Ac)(ε-DGA-DTX)]₃₂[ε-Lys(PEG₅₇₀)₂]₃₂

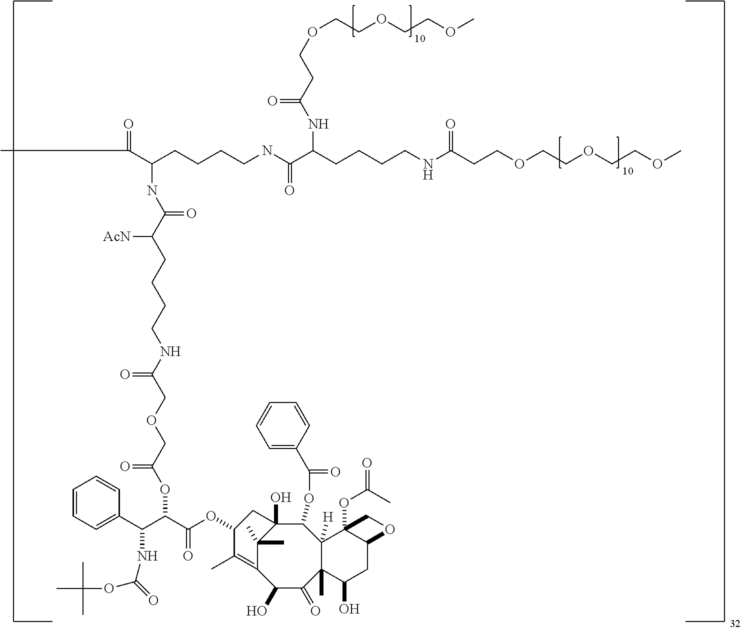

Prepared using Procedure C above, using BHALys[Lys]₃₂[α-Lys(α-Ac)(ε-NH₂. TFA)]₃₂[ε-Lys(PEG₅₇₀)₂]₃₂ (49 mg, 0.85 μmol) and DGA-DTX (31 mg, 34 μmol). Purification by SEC (sephadex, LH20, MeOH) provided 57 mg (80%) of the desired product as a white solid. HPLC (C8, gradient: 40-80% ACN/H₂O (1-7 min), 80% ACN (7-9 min), 80-40% ACN (9-11 min), 40% ACN (11-15 min), 10 mM ammonium formate) Rt (min)=8.85. ¹H NMR (300 MHz, CD₃OD) δ (ppm): 0.79-2.73 (m, 1698H), 3.06-3.29 (m, 179H), 3.35 (s, 184H), 3.36-3.92 (m, 2848H), 3.95-4.60 (m, 332H), 5.01 (br s, 32H), 5.20-5.52 (m, 77H), 5.64 (br s, 30H), 6.13 (br s, 27H), 7.14-7.34 (m, 39H), 7.34-7.52 (m, 104H), 7.52-7.76 (m, 87H), 8.02-8.24 (m, 57H). Theoretical molecular weight of conjugate: 83.3 kDa. ¹H NMR indicates 27 DTX/dendrimer. Actual molecular weight is approximately 78.8 kDa (28% DTX by weight).

### Example 12

Preparation of BHALys[Lys]₃₂[α-Glu-PTX]₃₂[ε-PEG₂₃₀₀]₃₂

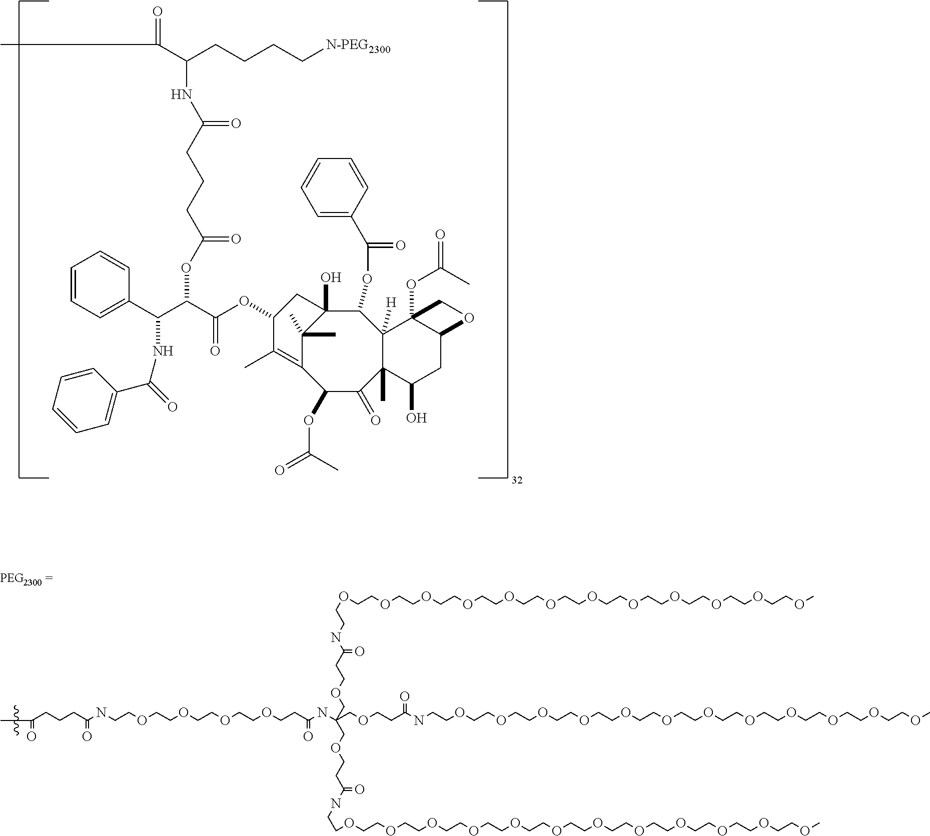

Prepared using Procedure C above, using Glu-PTX (300 mg, 371 μmol) and BHALys[Lys]₁₆[Lys (α-NH₂·TFA) (ε-PEG₂₃₀₀)]₃₂ (22.0 mg, 0.26 μmol). Purification by preparative HPLC (Rt=28 min) provided 12 mg (41%) of the desired dendrimer. ¹H NMR (CD₃OD): δ 0.78-2.80 (m, 1785H), 2.96-3.23 (m, 120H), 3.35-3.45 (m, 567H), 3.46-3.94 (m, 5610H), 4.04-4.47 (m, 167H), 4.48-4.65 (m, 88H), 5.50 (m, 29H), 5.64 (m, 24H), 5.85 (m, 27H), 6.10 (m, 26H), 6.46 (m, 20H), 7.26 (m, 66H), 7.36-8.00 (m, 407H), 8.12 (s, 53H). Theoretical molecular weight of conjugate: 112.4 kDa. 1H NMR indicates 25 PTX/dendrimer. Actual molecular weight is approximately 105 kDa (20% PTX by weight).

### Example 13

Preparation of BHALys[Lys]₃₂[α-Glu-GEM]₃₂[ε-PEG₁₁₀₀]₃₂ GEM=gemcitabine

### (a) Preparation of N,O-di-BOC-GEM-Glu

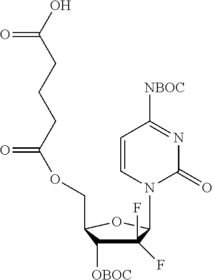

To a stirred mixture of N,O-diBoc gemicitabine (Guo, Z.; Gallo, J. M. Selective Protection of 2′,2′Difluorodeoxycytidine J. Org. Chem, 1999, 64, 8319-8322) (200 mg, 0.43 mmol) in DMF (2 mL) at 0° C. was added DIPEA (0.4 mL, 2.15 mmol) and glutaric anhydride (100 mg, 0.86 mmol). The reaction was allowed to warm up to ambient temperature over 1 hour, then stirred for a further 3 hours. The DMF was then removed in vacuo and residue was taken up in ethyl acetate (20 mL). This mixture was then washed with NaHCO₃ (10%, 2×10 mL), water (2×20 mL) and brine (20 mL). The organic phase was then dried (Na₂SO₄), filtered and concentrated under reduced pressure. The crude was then purified by silica gel chromatography (DCM/Methanol) providing 130 mg (54%) of the desired product as a white solid. LCMS (C18, gradient: 20-60% ACN/H₂O (1-7 min), 60% ACN (7-9 min), 60-20% ACN (9-11 min), 20% ACN (11-15 min), 0.1% TFA, Rt (min)=10.8 min. ESI (+ve) observed [M+H]⁺=578. Calculated for C₂₄H₃₂N₃F₂O₁₁=576.20 Da. ¹H NMR (CDCl₃): δ 1.51 (s, 18H), 2.01-1.88 (m, 2H), 2.55-2.4 (m, 2H), 2.75-2.64 (m, 2H), 4.46-4.38 (m, 3H), 5.15-5.10 (m, 1H), 6.46-6.30 (m, 1H), 7.36-7.50 (d, J=7.8 Hz, 1H), 7.6-7.79 (d, J=7.8 Hz, 1H).

(b) Preparation of BHALys[Lys]₃₂[@-Glu-GEM]₃₂[ε-PEG₁₁₀₀]₃₂

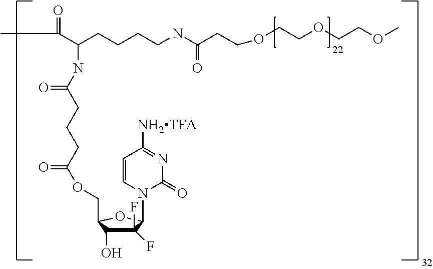

Prepared using Procedure C above, using BHALys[Lys]₁₆[Lys (α-NH₂·TFA)(ε-PEG)₁₁₀₀]₃₂ (40 mg, 1.03 mmol) and N,O-di-Boc-GEM-Glu (28 mg, 49 μmol). Purification by SEC (PD-10 desalting column, GE Healthcare, 17-0851-01, sephadex G-25 medium) provided 20 mg of material. The solid was taken up in TFA/DCM (1:1, 2 mLs) and stirred for 3 hours at room temperature. The volatiles were removed in vacuo and the residue taken up in water and freeze dried, providing 18 mg (47%) of white powder. HPLC (C8, gradient: 40-80% ACN/H₂O (1-7 min), 80% ACN (7-9 min), 80-40% ACN (9-11 min), 40% ACN (11-15 min), 0.1% TFA), Rt (min)=6.06. ¹H NMR (CD₃OD): δ 0.89-2.1 (m, 456H), 2.1-2.7 (m, 185H), 2.9-3.2 (m, 90H), 3.2-3.3 (m, 191H), 3.44-4.12 (m, 2650H), 4.14-4.70 (m, 160H), 5.8-6.0 (m, 28H), 6.2-6.4 (m, 28H), 7.05-7.15 (s, 11H), 7.5-7.7 (m, 24H). Theoretical molecular weight of conjugate: 59.2 kDa. ¹H NMR indicates 26 GEM/dendrimer. Actual molecular weight is approximately 52.3 kDa (15% GEM by weight).

### Example 14

(a) Preparation of BHALys[Lys]₃₂[α-GGG-Boc]₃₂[ε-PEG₁₁₀₀]₃₂

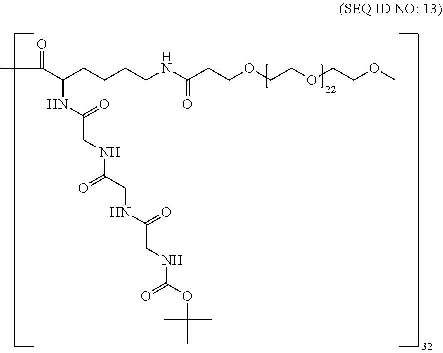

To a magnetically stirred solution of Boc-GGG-OH (28 mg, 93.2 μmol) and PyBOP (48 mg, 93.2 μmol) in DMF (1 mL) at room temperature was added a solution of BHALys[Lys]₃₂[α-NH₂·TFA]₃₂[ε-PEG₁₁₀₀]₃₂ (100 mg, 2.33 μmol) and DIPEA (51 μL, 298.24 μmol) in DMF (2.6 mL). The mixture was stirred at room temperature for 18 h and then concentrated under reduced pressure. The residue was dissolved in MeOH (1 mL) and purified by SEC (Sephadex, LH-20, MeOH). The appropriate fractions, as judged by HPLC, were combined and concentrated to provide 98 mg of product as a clear, colourless oil. The latter was dissolved in MQ water and lyophilised to give 98 mg (87%) of product as a colourless resin.LCMS (C8, gradient: 5-80% ACN/H₂O (1-7 min), 80% ACN (7-12 min), 80-5% ACN (12-13 min), 5% ACN (13-15 min), 0.1% TFA) Rt (min)=8.63. ¹H NMR (300 MHz, CD₃OD) δ (ppm): 1.15-2.01 (m, 693H), 2.46 (br s, 57H), 3.18 (br s, 101H), 3.35 (s, 53H), 3.36 (s, 84H), 3.38-4.04 (m, 2990H), 4.30 (br s, 63H), 6.17 (br s, 1H), 7.29 (br s, 9H). ¹H NMR indicates ca. 32 Boc-GGG/dendrimer. Molecular weight is approximately 48.5 kDa.

(b) Preparation of BHALys[Lys]₃₂[α-GGG-NH₂·TFA]₃₂[ε-PEG₁₁₀₀]₃₂

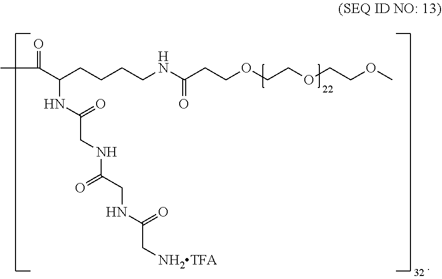

To a magnetically stirred mixture of BHALys[Lys]₃₂[α-GGG-Boc]₃₂[ε-PEG₁₁₀₀]₃₂ (98 mg, 2.02 μmol) in CH₂Cl₂ (1 mL) at room temperature was added a solution of TFA in CH₂Cl₂ (1:1, 2 mL). After 18 hours at room temperature the volatiles were removed. The resulting residue was dissolved in MQ water (15 mL) and concentrated. This procedure was repeated once more. The residue was then dissolved in MQ water (12.5 mL) and purified by SEC (PD-10, MQ water). The appropriate fractions were combined and lyophilised to provide 92 mg (94%) of desired material as a clear, colourless oil. HPLC (C8, gradient: 5-80% ACN/H₂O (1-7 min), 80% ACN (7-12 min), 80-5% ACN (12-13 min), 5% ACN (13-15 min), 0.1% TFA) Rt (min)=7.94. ¹H NMR (300 MHz, CD₃OD) δ (ppm): 1.19-2.05 (m, 351H), 2.47 (br s, 58H), 3.18 (br s, 105H), 3.36 (s, 89H), 3.38-4.15 (m, 2990H), 4.31 (br s, 72H), 6.17 (br s, 1H), 7.30 (br s, 9H). 1H NMR indicates ca. 32 GGG-NH₂·TFA/dendrimer. Molecular weight is approximately 48.6 kDa.

(c) Preparation of BHALys[Lys]₃₂[α-GGG-Glu-DTX]₃₂[ε-PEG₁₁₀₀]₃₂

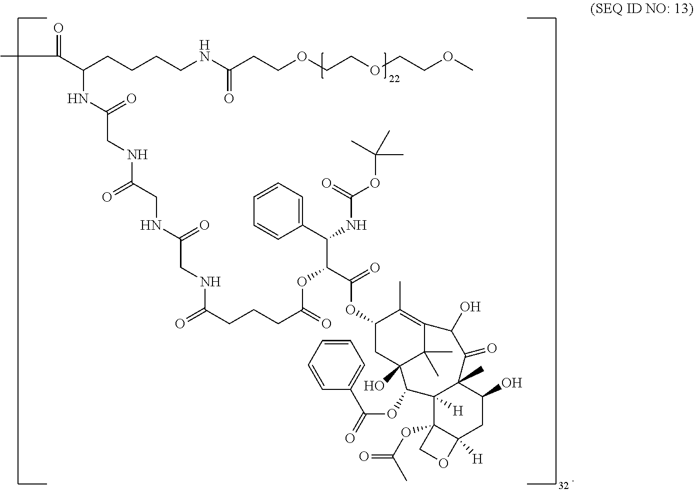

Prepared using Procedure C above, using BHALys[Lys]₃₂[α-GGG-NH₂. TFA]₃₂[ε-PEG₁₁₀₀]₃₂ (75 mg, 1.53 μmol) and Glu-DTX (56 mg, 61.2 μmol). Purification by SEC (Sephadex, LH-20, MeOH) provided 96 mg (92%) of product as a white solid. HPLC (C8, gradient: 5-80% ACN/H₂O (1-7 min), 80% ACN (7-12 min), 80-5% ACN (12-13 min), 5% ACN (13-15 min), 0.1% TFA) Rt (min)=10.08. ¹H NMR (300 MHz, CD₃OD) δ (ppm): 0.75-2.02 (m, 985H), 2.02-2.64 (m, 309H), 2.92-3.17 (m, 53H), 3.25 (s, 89H), 3.26-4.00 (m, 3070H), 4.00-4.40 (m, 174H), 4.82-5.00 (m, 44H), 5.04-5.39 (m, 87H), 5.54 (br s, 27H), 6.01 (br s, 22H), 7.03-7.67 (m, 227H), 7.92-8.10 (m, 49H). Theoretical molecular weight of conjugate: 73.9 kDa. ¹H NMR indicates 32 GGG and 26 DTX/dendrimer. Actual molecular weight is approximately 68.5 kDa (31% DTX by weight).

### Example 15

(a) Preparation of BHALys[Lys]₃₂[α-GFLG-Boc]₃₂[ε-PEG₁₁₀₀]₃₂

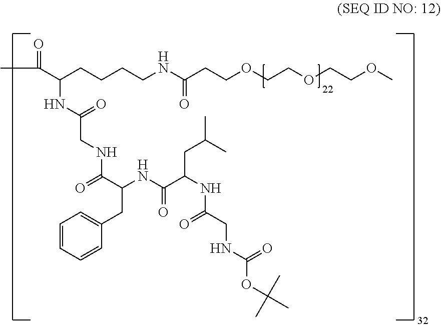

To a magnetically stirred solution of Boc-GLFG-OH (32 mg, 65.2 μmol) and PyBOP (34 mg, 65.2 μmol) in DMF (1 mL) at room temperature was added a solution of BHALys[Lys]₃₂[α-NH₂. TFA]₃₂[ε-PEG₁₁₀₀]₃₂ (70 mg, 1.63 μmol) and DIPEA (36 μL, 208.64 μmol) in DMF (1.5 mL). The mixture was stirred at room temperature for 18 h and then concentrated under reduced pressure. The residue was dissolved in MeOH (1 mL) and purified by SEC (Sephadex, LH-20, MeOH). The appropriate fractions, as judged by HPLC, were combined and concentrated to provide 77 mg (88%) of product as a clear, colourless oil. HPLC (C8, gradient: 5-80% ACN/H₂O (1-7 min), 80% ACN (7-12 min), 80-5% ACN (12-13 min), 5% ACN (13-15 min), 0.1% TFA) Rt (min)=9.14. ¹H NMR (300 MHz, CD₃OD) δ (ppm): 0.63-1.06 (m, 211H), 1.06-2.11 (m, 789H), 2.32-2.62 (m, 61H), 2.88-3.28 (m, 148H), 3.36 (s, 95H), 3.37-4.00 (m, 2920H), 4.17-4.69 (m, 132H), 7.23 (br s, 140H). ¹H NMR indicates ca. 30 Boc-GLFG/dendrimer. Molecular weight is approximately 53.8 kDa.

(b) Preparation of BHALys[Lys]₃₂[α-GFLG-NH₂. TFA]₃₂[ε-PEG₁₁₀₀]₃₂

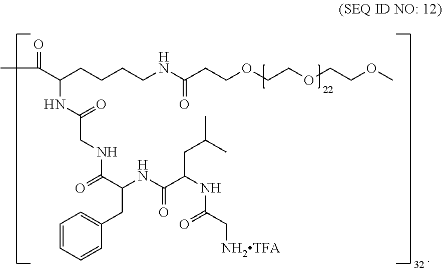

To a magnetically stirred mixture of BHALys[Lys]₃₂[α-GFLG-Boc]₃₂[ε-PEG₁₁₀₀]₃₂ (77 mg, 1.43 μmol) in CH₂Cl₂ (1 mL) at room temperature was added a solution of TFA in CH₂Cl₂ (1:1, 2 mL). After 3 hours at room temperature the volatiles were removed. The resulting residue was dissolved in MQ water (15 mL) and concentrated. This procedure was repeated once more. The residue was then dissolved in MQ water (15 mL) and lyophilised to provide 76 mg (99%) of desired material as a yellowish resin.HPLC (C8, gradient: 5-80% ACN/H₂O (1-7 min), 80% ACN (7-12 min), 80-5% ACN (12-13 min), 5% ACN (13-15 min), 0.1% TFA) Rt (min)=8.08. ¹H NMR (300 MHz, CD₃OD) δ (ppm): 0.75-1.04 (m, 197H), 1.10-2.09 (m, 480H), 2.45 (m, 56H), 2.88-3.29 (m, 146), 3.35 (s, 90H), 3.37-4.05 (m, 2920H), 4.17-4.69 (m, 133H), 7.66 (s, 159H). Theoretical molecular weight of conjugate: 68.9 kDa. ¹H NMR indicates ca. 30 GFLG-NH₂·TFA/dendrimer (SEQ ID NO: 9). Molecular weight is approximately 54.1 kDa.

(c) Preparation of BHALys[Lys]₃₂[α-GFLG-Glu-DTX]₃₂[ε-PEG₁₁₀₀]₃₂

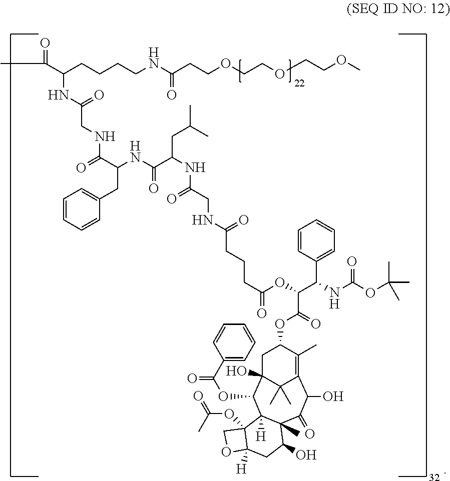

Prepared using Procedure C above, using BHALys[Lys]₃₂[α-GFLG-NH₂·TFA]₃₂[ε-PEG₁₁₀₀]₃₂ (61 mg, 1.13 μmol) and Glu-DTX (42 mg, 45.60 μmol). Purification by SEC (Sephadex, LH-20, MeOH) provided 68 mg (85%) of product as a white solid. HPLC (C8, gradient: 5-80% ACN/H₂O (1-7 min), 80% ACN (7-12 min), 80-5% ACN (12-13 min), 5% (ACN 13-15 min), 0.1% TFA) Rt (min)=10.16. ¹H NMR (300 MHz, CD₃OD) δ (ppm): 0.85 (s, 173H), 0.99-2.13 (m, 1153H), 2.15-2.62 (m, 312H), 2.91-3.27 (m, 128H), 3.35 (s, 93), 3.36-4.00 (m, 2970H), 4.05-4.68 (m, 237H), 4.94-5.07 (m, 32H), 5.15-5.47 (m, 76H), 5.52-5.76 (m, 24H), 5.97-6.26 (s, 21H), 6.99-7.77 (m, 380H), 7.98-8.24 (m, 48H). Theoretical molecular weight of conjugate: 80.4 kDa. 1H NMR indicates 30 GLFG and 22 DTX/dendrimer. Actual molecular weight is approximately 70.6 kDa (25% DTX by weight).

### Example 16

Preparation of BHALys[Lys]₃₂[α-GILGVP-Glu-DTX]₃₂[ε-PEG₁₁₀₀]₃₂

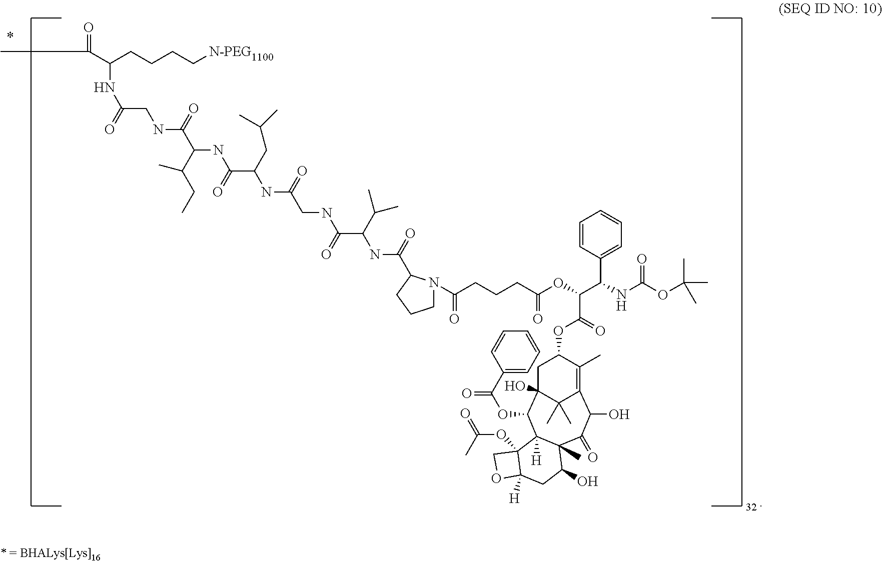

Prepared using Procedure C above, using BHALys[Lys]₃₂[ε-GILGVP-NH·TFA]₃₂[α-PEG₁₁₀₀]₃₂ (52 mg, 0.86 μmol) and Glu-DTX (34 mg, 36 μmol). Purification by SEC (sephadex, LH20, MeOH) provided 59 mg (80%) of desired material as a hygroscopic colourless solid. HPLC (C8, gradient: 5-80% ACN/H₂O (1-7 min), 80% ACN (7-12 min), 80-5% ACN (12-13 min), 5% ACN (13-15 min), 0.1% TFA buffer) Rt (min)=10.45. ¹H NMR (300 MHz, CD₃OD) δ (ppm): 0.84-1.91 (m, 1808H), 2.41 (s, 287H), 3.12-3.20 (m, 106H), 3.35 (bd, 166H), 3.37-3.90 (m, 2800H), 4.10-4.40 (bm, 194H), 4.53 (s, 88H), 4.98-5.03 (m, 35H), 5.24-5.40 (m, 80H), 5.60-5.68 (m, 26H), 6.08-6.16 (m, 21H), 7.25-7.88 (m, 288H), 8.08-8.16 (m, 86H). Theoretical molecular weight of conjugate: 85.6 kDa. ¹H NMR indicates 30 DTX/dendrimer. Actual molecular weight is approximately 83.2 kDa (29% DTX by weight).

### Example 17

Preparation of BHALys[Lys]₃₂[α-GILGVP-Glu-DTX]₃₂[ε-t-PEG₂₃₀₀]₃₂

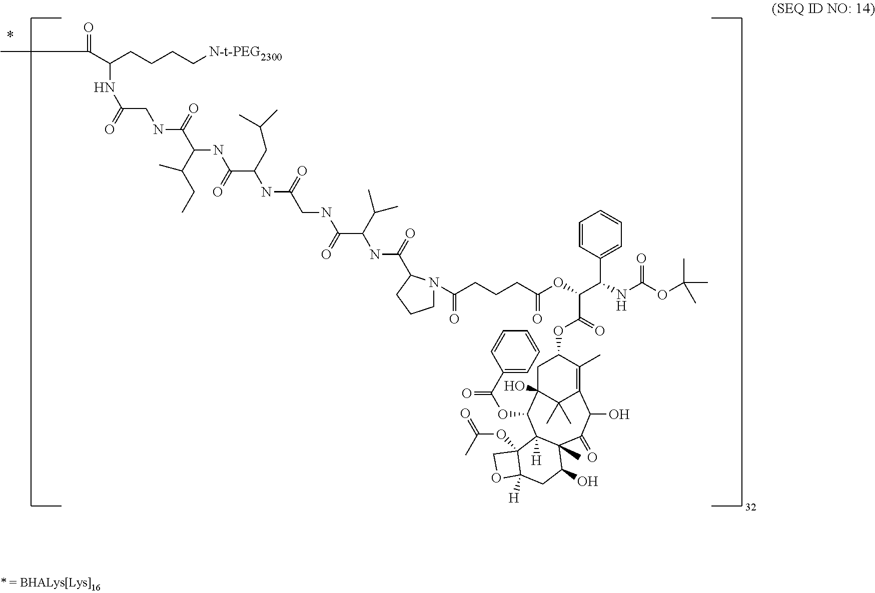

Prepared using Procedure C above, using BHALys[Lys]₃₂[α-GILGVP-NH₂. TFA]₃₂[ε-t-PEG₂₃₀₀]₃₂ (59 mg, 0.57 μmol) and Glu-DTX (23 mg, 25 μmol) and PyBOP (13 mg, 25 μmol) Purification by SEC (sephadex, LH20, MeOH) provided 65 mg (89%) of desired material as a hygroscopic colourless solid. HPLC (C8, gradient: 5-80% ACN/H₂O (1-7 min), 80% ACN (7-12 min), 80-5% ACN (12-13 min), 5% ACN (13-15 min), 0.1% TFA buffer) Rt (min)=9.22. ¹H NMR (300 MHz, CD₃OD) δ (ppm): 0.86-2.50 (m, 2622H), 3.12-3.20 (m, 80H), 3.35-3.88 (m, 5540H), 4.18-4.30 (bm, 263H), 4.50-4.58 (m, 149H), 4.96-5.04 (m, 42H), 5.24-5.38 (m, 77H), 5.62-5.68 (m, 29H), 6.08-6.14 (m, 28H), 7.25-7.70 (m, 234H), 8.10-8.15 (m, 63H). Theoretical molecular weight of conjugate: 127.3 kDa. ¹H NMR indicates 27 DTX/dendrimer. Actual molecular weight is approximately 123.7 kDa (18% DTX by weight).

### Example 18

Preparation of BHALys[Lys]₃₂[α-PEG₁₁₀₀]₃₂[ε-TDA-DTX]₃₂

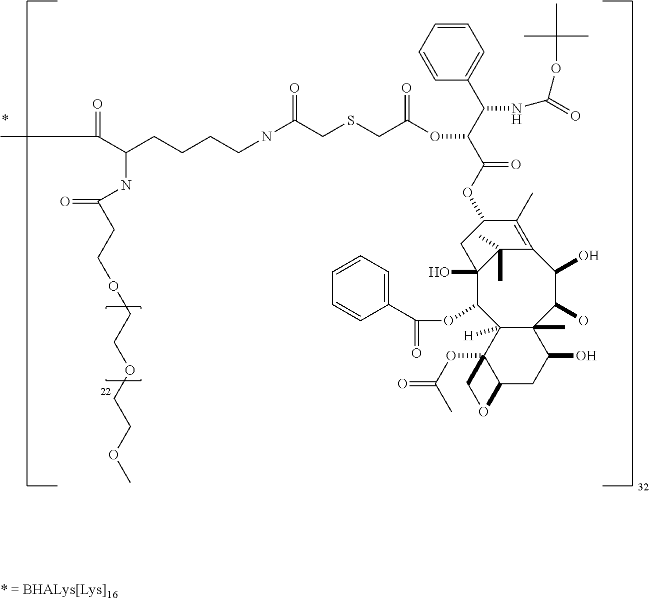

Prepared using Procedure C above, using BHALys[Lys]₃₂[ε-NH₂. TFA]₃₂[α-PEG₁₁₀₀]₃₂ (57.5 mg, 1.34 μmol) and TDA-DTX (52.3 mg, 56 μmol). Purification by SEC (sephadex, LH20, MeOH) provided 70 mg (92%) of desired material as a hygroscopic colourless solid. HPLC (C8, gradient: 5-80% ACN/H₂O (1-7 min), 80% ACN (7-12 min), 80-5% ACN (12-13 min), 5% ACN (13-15 min), 0.1% TFA buffer) Rt (min)=9.89. ¹H NMR (300 MHz, CD₃OD) δ (ppm): 1.06-1.95 (m, 784H), 2.36-2.55 (m, 168H), 3.04-3.23 (m, 48H), 3.33 (s, 84H), 3.35-3.89 (m, 2800H), 4.13-4.40 (m, 118H), 5.23-5.40 (m, 72H), 5.59-5.66 (m, 24H), 6.06-6.16 (m, 23H), 7.25-7.65 (m, 234H), 8.10-8.12 (m, 52H). Theoretical molecular weight of conjugate: 68.9 kDa. ¹H NMR indicates 27 DTX/dendrimer. Actual molecular weight is approximately 64.4 kDa (34% DTX by weight).

### Example 19

Preparation of BHALys[Lys]₃₂[α-TDA-DTX]₃₂[ε-PolyPEG₂₀₀₀]₃₂

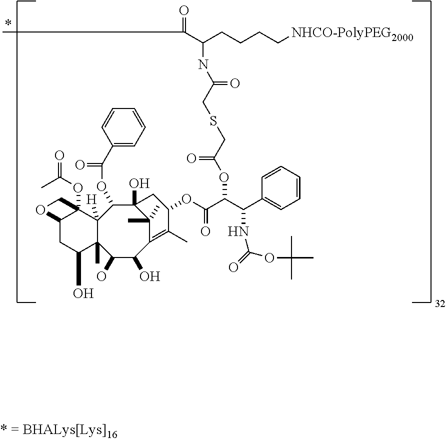

Prepared using Procedure C above, using BHALys[Lys]₃₂[α-NH₂. TFA]₃₂[ε-PEG₂₀₀₀]₃₂ (88.6 mg, 1.2 μmol) and TDA-DTX (49.3 mg, 52 μmol). Purification by SEC (sephadex, LH20, MeOH) provided 95 mg (80%) of desired material as a hygroscopic colourless solid. HPLC (C8, gradient: 45-85% ACN/H₂O (1-7 min), 85% ACN (7-12 min), 85-45% ACN (12-13 min), 45% ACN (13-15 min), 0.1% TFA buffer) Rf (min)=6.29 min. ¹H NMR (300 MHz, CD₃OD) δ (ppm): 0.82-1.96 (m, 2076H), 2.36-2.54 (m, 314H), 3.10-3.24 (m, 125H), 3.35-3.89 (m, 6300H), 4.96-5.04 (m, 35H), 5.25-5.45 (m, 79H), 5.60-5.70 (m, 29H), 6.06-6.18 (m, 24H), 7.20-7.75 (m, 269H), 8.06-8.16 (m, 52H). Theoretical molecular weight of conjugate: 101.1 kDa. ¹H NMR indicates 27 DTX/dendrimer. Actual molecular weight is approximately 95.5 kDa (23% DTX by weight). Particle sizing using Dynamic Light Scattering shows a range of concentration dependent averages of 10.9-15.5 nm.

### Example 20

Preparation of BHALys[Lys]₃₂[α-DGA-testosterone]₃₂[ε-PEG₁₁₀₀]₃₂

### (a) Preparation of DGA-Testosterone

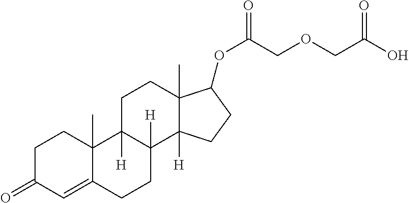

Prepared using Procedure B above, using testosterone (256 mg, 0.88 mmol), pyridine (10 mL) as the solvent and diglycolic anhydride (1.02 g, 8.8 mmol) as the linker. Purification by preparatory HPLC (BEH 300 Waters XBridge C18, 5 μM, 30×150 mm, 40-90% ACN/water, no buffer, RT=62 min) to give the desired compound 241 mg (67% yield) as an off white hygroscopic solid. LCMS (C8, gradient: 40-90% ACN/H₂O (1-7 min), 90% ACN (7-9 min), 90-40% ACN (9-11 min), 40% ACN (11-15 min), 0.1% TFA) Rt (min)=5.61. ESI (−ve) observed [M−H]⁻=403.29. Calculated for C₂₃H₃₁O₆=403.21 Da. ¹H NMR (300 MHz, CD₃OD) δ (ppm) 0.88 (s, 3H, CH₃), 0.93-1.23 (m, 3H), 1.24 (s, 3H, CH₃), 1.25-2.58 (br m, 16H), 4.18 (s, 2H, CH₂), 4.23 (s, 2H, CH₂), 4.70 (m, 1H, CH), 5.71 (s, 1H, CH).

(b) Preparation of BHALys[Lys]₃₂[α-DGA-Testosterone]₃₂[ε-PEG₁₁₀₀]₃₂

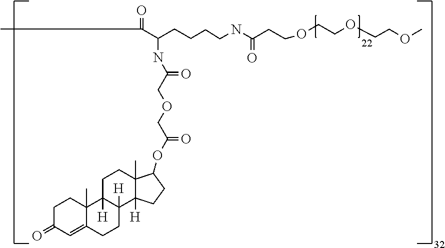

Prepared using Procedure C above, using BHALys[Lys]₃₂ (α-NH₂·TFA)₃₂(ε-PEG₁₁₀₀)₃₂(30 mg, 0.75 μmol) and DGA-Testosterone (19 mg, 47 μmol). Purification by SEC (LH20, eluent: methanol) provided 15 mg (39%) as an off-white solid. HPLC (C8, gradient: 30-80% ACN/H₂O (1-7 min), 80% ACN (7-9 min), 80-30% ACN (9-11 min), 30% ACN (11-15 min), 10 mM ammonium formate) Rt (min)=9.41. ¹H NMR (300 MHz, CD₃OD) δ (ppm) 0.79 (s, 80H, CH₃), 0.81-2.42 (br m, 1101H), 3.08 (m, 116H, CH₂), 3.26 (s, 98H, CH₂), 3.37-3.81 (m, 2800H, CH₂), 3.95-4.47 (m, 173H, CH), 4.61 (m, 29H, CH), 5.62 (s, 29H, CH), 6.08 (m, 1H, CH), 7.17 (m, 10H, ArH). Theoretical molecular weight of conjugate: 52.4 kDa. ¹H NMR indicates 29 testosterone/dendrimer. Actual molecular weight is approximately 51.2 kDa (16% testosterone by weight).

### Example 21

Preparation of BHALys[Lys]₃₂[α-DGA-Testosterone]₃₂[ε-PEG₅₇₀]₃₂

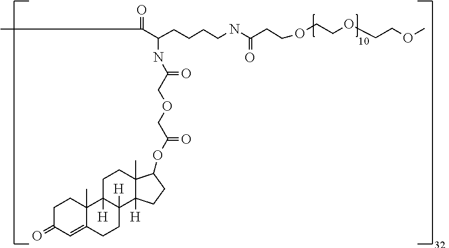

Prepared using Procedure C above, using BHALys[Lys]₃₂ (α-NH₂·TFA)₃₂(ε-PEG₅₇₀) 32 (40 mg, 1.33 μmol) in DMF (2 mL) and DGA-Testosterone (43 mg, 106 μmol). Purification by SEC (LH20, eluent: methanol) provided 22.1 mg (40% yield) as a white hygroscopic solid. HPLC (C8, gradient: 30-80% ACN/H₂O (1-7 min), 80% ACN (7-9 min), 80-30% ACN (9-11 min), 30% ACN (11-15 min), 10 mM ammonium formate) Rt (min)=9.99. ¹H NMR (300 MHz, CD₃OD) δ (ppm) 0.89 (s, 96H, CH₃), 0.90-2.63 (br m, 1214H), 3.36 (m, 125H, CH₂), 3.36 (s, 100H, CH₃), 3.45-3.97 (m, 1472H, CH₂), 4.05-4.62 (m, 218H), 4.71 (m, 37H, CH), 5.72 (s, 31H, CH), 6.18 (m, 1H, CH), 7.17 (m, 10H, ArH). Theoretical molecular weight of conjugate: 42.5 kDa. ¹H NMR indicates 31 testosterone/dendrimer. Actual molecular weight is approximately 42.1 kDa (21% testosterone by weight).

### Example 22

Preparation of BHALys[Lys]₃₂[α-Glu-testosterone]₃₂[ε-PEG₁₁₀₀]₃₂

### (a) Preparation of Glu-Testosterone

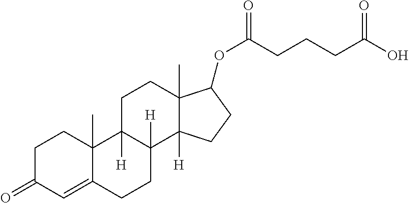

Prepared using Procedure B above, using testosterone (100 mg, 0.35 mmol), pyridine (6 mL) as the solvent and glutaric anhydride (396 mg, 3.5 mmol) as the linker. Purification by preparatory HPLC (BEH 300 Waters XBridge C18, 5 μM, 30×150 mm, 40-90% ACN/water, no buffer, RT=62 min) to give the desired compound 86 mg (86%) as an off white hygroscopic solid. LCMS (C8, gradient: 40-90% ACN/H₂O (1-7 min), 90% ACN (7-9 min), 90-40% ACN (9-11 min), 40% ACN (11-15 min), 0.1% TFA) Rt (min)=6.40. ESI (+ve) observed [M+H]⁺=403.29. Calculated for C₂₄H₃₅O₅=403.25 Da. ¹H NMR (300 MHz, CD₃OD) δ (ppm) 0.89 (s, 3H, CH₃), 0.93-1.23 (m, 3H), 1.24 (s, 3H, CH₃), 1.36-2.57 (br m, 22H), 4.62 (m, 1H, CH), 5.71 (s, 1H, CH).

(b) Preparation of BHALys[Lys]₃₂[α-Glu-Testosterone]₃₂[ε-PEG₁₁₀₀]₃₂

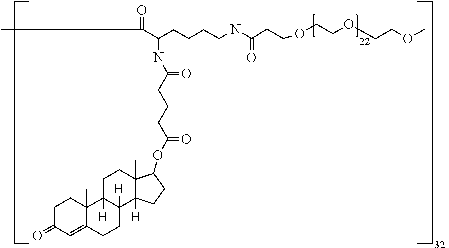

Prepared using Procedure C above, using BHALys[Lys]₃₂ (α-NH₂·TFA)₃₂(ε-PEG₁₁₀₀)₃₂(30 mg, 0.75 μmol) in DMF (2 mL) and Glu-Testosterone (19 mg, 47 μmol). Purification by SEC (LH20, eluent: methanol) provided 18.1 mg (47%) of the desired product as an off-white solid. HPLC (C8, gradient: 40-80% ACN/H₂O (1-7 min), 80% ACN (7-9 min), 80-40% ACN (9-11 min), 40% ACN (11-15 min), 10 mM ammonium formate) Rt (min)=7.22. ¹H NMR (300 MHz, CD₃OD) δ (ppm) 0.88 (s, 87H, CH₃), 0.89-2.61 (br m, 1225H), 3.17 (m, 110H, CH₂), 3.36 (s, 101H, CH₃), 3.46-3.98 (m, 2800H, CH₂), 4.34 (m, 59H, CH), 4.61 (m, 30H, CH), 5.72 (s, 29H, CH), 6.18 (m, 1H, CH), 7.28 (m, 12H, ArH). Theoretical molecular weight of conjugate: 52.3 kDa. ¹H NMR indicates 29 testosterone/dendrimer. Actual molecular weight is approximately 51.1 kDa (16% testosterone by weight).

### Example 23

Preparation of BHALys[Lys]₃₂[α-Glu-Testosterone]₃₂[ε-PEG₅₇₀]₃₂

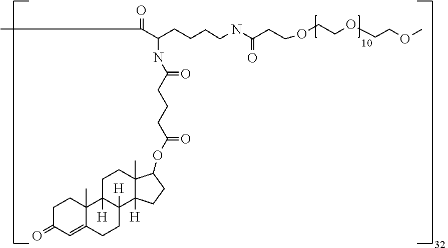

Prepared using Procedure C above, using BHALys[Lys]₃₂ (α-NH₂·TFA)₃₂(ε-PEG₅₇₀)₃₂(30 mg, 1 μmol) in DMF (2 mL) and Example 22 (a), Glu-Testosterone (26 mg, 64 μmol). Purification by SEC (LH20, eluent: methanol) provided 19.8 mg (47% yield) of the desired product as a white solid product. HPLC (C8, gradient: 40-80% ACN/H₂O (1-7 min), 80% ACN (7-9 min), 80-40% ACN (9-11 min), 40% ACN (11-15 min), 10 mM ammonium formate) Rt (min)=8.93. ¹H NMR (300 MHz, CD₃OD) δ (ppm): 0.88 (s, 96H, CH₃), 0.89-2.59 (br m, 1423H), 3.16 (m, 127H, CH₂), 3.26 (m, 135H, CH₃), 3.65-3.92 (m, 1472H, CH₂), 4.24 (m, 66H, CH), 4.52 (m, 39H, CH), 5.62 (s, 32H, CH), 6.09 (m, 1H, CH), 7.19 (m, 10H, ArH). Theoretical molecular weight of conjugate: 42.5 kDa. ¹H NMR indicates 32 testosterone/dendrimer. Actual molecular weight is approximately 42.5 kDa (21% testosterone by weight).

### Example 24

Preparation of BHALys[Lys]₃₂[α-Glu-SB]₃₂[ε-PEG₁₁₀₀]₃₂ SB=Salbutamol

### (a) Preparation of Glu-SB

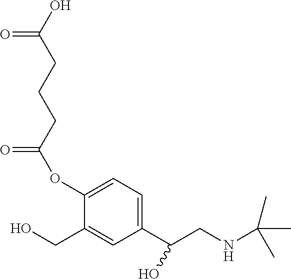 [SMILES: CC(C)(C)NCC(O)c1ccc(OC(=O)CCCC(=O)O)c(CO)c1]

Prepared using Procedure B above, using SB (100 mg, 0.42 mmol) and glutaric anhydride (62 mg, 0.54 mmol) as the linker. Preparative HPLC (BEH 300 Waters XBridge C18, 5 M, 30×150 mm, gradient: 5% ACN/H₂O (1-5 min), 5-60% ACN (5-40 min), 60% ACN (40-45 min), 60-5% ACN (45-50 min), 5% ACN (50-60 min), 0.1% TFA, Rt=27 min) provided 50 mg (34%) of the desired product as a white solid. HPLC (C18, gradient: 5-60% ACN/H₂O (1-10 min), 60% ACN (10-11 min), 60-5% ACN (11-13 min), 5% ACN (13-15 min), 10 mM ammonium formate) Rt (min)=6.67. ESI (+ve) observed [M+H]⁺=354. Calculated for C₁₈H₂₇NO₆=353.18 Da. ¹H NMR (300 MHZ, CD₃OD) δ (ppm): 1.41 (s, 9H), 1.92 (t, J=7.2 Hz, 2H), 2.37 (t, J=7.5 Hz, 2H), 2.45 (t, J=7.2 Hz, 2H), 3.01-3.18 (m, 2H), 5.18 (s, 2H), 6.87 (d, J=8.4 Hz, 1H), 7.27 (dd, J=8.4 and 2.1 Hz, 1H), 7.36 (d, J=2.4 Hz, 1H).

(b) Preparation of BHALys[Lys]₃₂[α-Glu-SB]₃₂[ε-PEG₅₇₀]₃₂

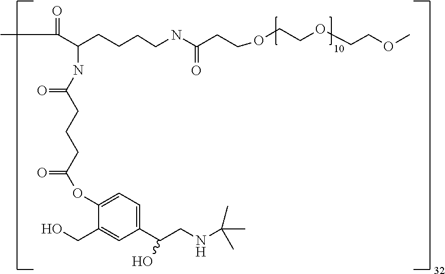

Prepared using Procedure C above, using BHA [Lys]₃₂[α-NH₂. TFA]₃₂[ε-PEG₅₇₀]₃₂ (26 mg, 0.86 μmol) and Glu-SB (17 mg, 48.2 μmol). Purification by SEC (sephadex, LH20, MeOH) provided 25 mg (76%) of desired material as a white solid. HPLC (C8, gradient: 40-80% ACN/H₂O (1-7 min), 80% ACN (7-9 min), 80-40% ACN (9-11 min), 40% ACN (11-15 min), 10 mM ammonium formate) Rt (min)=5.81. ¹H NMR (300 MHz, CD₃OD) δ (ppm): 1.03-2.02 (m, 738H), 2.25-2.58 (m, 180H), 2.97-3.29 (m, 167H), 3.40-3.94 (m, 1469H), 4.12-4.50 (m, 74H), 5.04 (s, 55H), 6.90 (d, J=8.1 Hz, 27H), 7.28 (d, J=8.1 Hz, 27H), 7.36 (m, 27H). Theoretical molecular weight of conjugate: 37.8 kDa. ¹H NMR indicates 27 salbutamol/dendrimer. Actual molecular weight is approximately 36.1 kDa (18% salbutamol by weight).

### Targeted Constructs

### Example 25

Preparation of 4-azidobenzamide-PEG₁₂-NEOEOEN[SuN(PN)₂][Lys]₁₆[Lys(α-PSSP-DTX) (ε-PEG₁₁₀₀)]₃₂
(a) Preparation of 4-azidobenzamide-PEG₁₂-NEOEOEN[SuN(PN)₂][Lys]₁₆[Lys (α-NHBOC)(ε-PEG₁₁₀₀)]₃₂

To a magnetically stirred solution of L-lysine-(α-NHBOC) (ε-PEG₁₁₀₀) (614 mg, 456 μmol) in anhydrous DMF (2.5 mL) was added PyBOP (246 mg, 473 μmol) followed by a solution of 4-azidobenzamide-PEG₁₂-NEOEOEN[SuN(PN)₂][Lys]₁₆[NH₂. TFA]₃₂ (91 mg, 10.6 μmol) and DIPEA (235 μL, 1.35 mmol) in anhydrous DMF (2.5 mL). After 16 hours at room temperature the reaction was concentrated in vacuo and the residue purified by ultrafiltration (PALL Minimate Cartridge 10 kDa membrane) to provide the target compound as an off-white sticky solid, 433 mg (86%). LCMS (C8 Waters X-Bridge, gradient: 40-90% ACN/H₂O (1-7 min), 90% ACN (7-9 min), 90-40% ACN (9-11 min), 40% ACN (11-15 min), 0.1% Formic Acid) Rt (min)=5.17.

(b) Preparation of 4-azidobenzamide-PEG₁₂-NEOEOEN[SuN(PN)₂][Lys]₁₆[Lys (α-NH₂. TFA) (ε-PEG₁₁₀₀)]₃₂

A solution of 4-azidobenzamide-PEG₁₂-NEOEOEN[SuN(PN)₂][Lys]₁₆[Lys (α-NHBOC) (ε-PEG₁₁₀₀)]₃₂ (431 mg, 9.10 μmol) in TFA/DCM (5 mL/7 mL) was left stirring for 4 h. After this time the reaction mixture concentrated and the resulting residue azeotroped with water (2×10 mL) to provide the target compound as a pale yellow oil, 435 mg (100%). LCMS (C18 Waters X-Bridge, gradient: 5-60% ACN/H₂O (1-10 min), 60% ACN/H₂O (10-14 min), 60-5% ACN/H₂O (14-16 min), 0.1% TFA) Rt=10.65. ¹H NMR (300 MHZ, D₂O) δ (ppm): 1.21-2.04 (m, 376H), 2.51-2.56 (m, 71H), 3.12-3.30 (m, 115H), 3.40 (s, 96H), 3.45-3.90 (m, 3077H), 3.91-4.42 (m, 62H), 7.25 (d, J 8.7 Hz, 2H), 7.88 (d, J 8.7 Hz, 2H).

(c) Preparation of 4-azidobenzamide-PEG₁₂-NEOEOEN[SuN(PN)₂][Lys]₁₆[Lys(α-PSSP-DTX)(ε-PEG₁₁₀₀)]₃₂

The construct was prepared using Procedure C above, using 4-azidobenzamide-PEG₁₂-NEOEOEN[SuN(PN)₂][Lys]₁₆[Lys (α-NH₂·TFA) (ε-PEG₁₁₀₀)]₃₂ (104 mg, 2.18 μmol) and DTX-PSSP (94 mg, 94.0 μmol). Purification by SEC provided 133 mg (97%) of the desired material as a pale yellow, viscous oil. LCMS (C18 Waters X-Bridge, gradient: 5-60% ACN/H₂O (1-10 min), 60% ACN/H₂O (10-11 min), 60-5% ACN/H₂O (11-13 min), 0.1% Formic acid) Rt (min)=7.59. ¹H NMR (300 MHZ, CD₃OD) δ (ppm): 0.88-2.05 (m, 1080H), 2.16-2.56 (m, 212H), 2.60-3.26 (m, 363H), 3.35-3.41 (m, 129H), 3.50-3.94 (m, 3110H), 4.00-4.60 (134H), 4.93-5.10 (m, 28H), 5.20-5.46 (m, 73H), 5.54-5.80 (m, 24H), 5.95-6.30 (m, 23H), 7.14-7.91 (m, 268H). Theoretical molecular weight of conjugate: 75.7 kDa. ¹H NMR indicates 26 DTX/dendrimer, therefore actual molecular weight is approximately 69.8 kDa (37% DTX by weight).

### Example 26

Preparation of biotin-triazolobenzamide-PEG₁₂-NEOEOEN[SuN(PN)₂][Lys]₁₆[Lys(α-PSSP-DTX)(ε-PEG₁₁₀₀)]₃₂

The construct was prepared using Procedure D above, using 4-azidobenzamide-PEG₁₂-NEOEOEN[SuN(PN)₂][Lys]₁₆[Lys(α-PSSP-DTX)(ε-PEG₁₁₀₀)]₃₂ (42.5 mg, 674 nmol) and biotin-alkyne (0.4 mg, 1.35 μmol). Purification by SEC provided the target compound as an off-white solid, 39 mg (91%). LCMS (C18 Waters X-Bridge, gradient: 5-60% ACN/H₂O (1-10 min), 60% ACN/H₂O (10-11 min), 60-5% ACN/H₂O (11-13 min), 0.1% Formic acid) Rt (min)=7.04. ¹H NMR (300 MHZ, CD₃OD) δ (ppm): 0.92-2.02 (m, 982H), 2.10-3.25 (m, 1027H), 3.35-3.42 (m, 128H), 3.49-3.98 (m, 3180H), 4.07-4.69 (m, 131H), 4.96-5.11 (m, 27H), 5.15-5.50 (m, 72H), 5.55-5.80 (m, 24H), 5.98-6.23 (m, 23H), 7.14-8.25 (m, 277H), 8.54-8.56 (m, 1H).

### Example 27

Preparation of LyP-1-triazolobenzamide-PEG₁₂-NEOEOEN[SuN(PN)₂][Lys]₁₆[Lys(α-PSSP-DTX)(ε-PEG₁₁₀₀)]₃₂

### LyP-1 (Supplied by AusPep Pty Ltd).

The construct was prepared using Procedure D above, using 4-azidobenzamide-PEG₁₂-NEOEOEN[SuN(PN)₂][Lys]₁₆[Lys(α-PSSP-DTX)(ε-PEG₁₁₀₀)]₃₂ (44.2 mg, 701 nmol) LyP-alkyne (185 μL of a 10 mg/mL solution in H₂O, 1.05 μmol). Purification by SEC provided a bright pink, sticky solid, 46 mg (102%), as a ca. mixture of 60:40 LyP-triazolobenzamide-PEG₁₂-NEOEOEN[SuN(PN)₂][Lys]₁₆[Lys(α-PSSP-DTX)(ε-PEG₁₁₀₀)]₃₂/4-azidobenzamide-PEG₁₂-NEOEOEN[SuN(PN)₂][Lys]₁₆[Lys(α-PSSP-DTX)(ε-PEG₁₁₀₀)]₃₂. LCMS (C8 Waters X-Bridge, gradient: 40-90% ACN/H₂O (1-7 min), 90% ACN (7-9 min), 90-40% ACN (9-11 min), 40% ACN (11-15 min), 0.1% Formic Acid) Rt (min)=6.07 (LyP-Dendrimer conjugate); 7.10 (Azido-Dendrimer starting material).

### Example 28

Preparation of deslorelin-triazolobenzamide-PEG₁₂-NEOEOEN[SuN(PN)₂][Lys]₁₆[Lys (α-PSSP-DTX)(ε-PEG₁₁₀₀)]₃₂

The construct was prepared using Procedure D above, using 4-azidobenzamide-PEG₁₂-NEOEOEN[SuN(PN)₂][Lys]₁₆[Lys(α-PSSP-DTX)(ε-PEG₁₁₀₀)]₃₂ (41.7 mg, 662 nmol) and deslorelin-alkyne (130 μL of a 10 mg/mL solution in H₂O, 993 nmol). Purification by SEC provided a pale yellow, sticky solid, 43 mg (100%), as a ca. mixture of 70:30 deslorelin-triazolobenzamide-PEG₁₂-NEOEOEN[SuN(PN)₂][Lys]₁₆[Lys(α-PSSP-DTX)(ε-PEG₁₁₀₀)]₃₂/4-azidobenzamide-PEG₁₂-NEOEOEN[SuN(PN)₂][Lys]₁₆[Lys(α-PSSP-DTX)(ε-PEG₁₁₀₀)]₃₂. LCMS (C8 Waters X-Bridge, gradient: 40-90% ACN/H₂O (1-7 min), 90% ACN (7-9 min), 90-40% ACN (9-11 min), 40% ACN (11-15 min), 0.1% Formic Acid) Rt (min)=6.42 (Deslorelin-Dendrimer conjugate); 7.11 (Azido-Dendrimer starting material).

### Example 29

### Preparation Antibody-Dendrimer Conjugation Using Streptavidin as a Joining Unit

To a solution of Alexa Fluor 750 Streptavidin (Av) (0.1 μg/mL) in phosphate-buffered saline (PBS, 2 mL) was added Abcam #ab24293 Anti-EGFR antibody biotin (Ab) (30 μL of 10 μg/mL stock solution). To this reaction solution was added a solution of biotin-triazolobenzamide-PEG₁₂-NEOEOEN[SuN(PN)₂][Lys]₁₆[Lys(α-PSSP-DTX)(ε-PEG₁₁₀₀)]₃₂(DTX-D) in PBS (5 μL of 1.0 μg/mL stock solution). The mixture was left stirring for 10 s and the above procedure of adding Ab and DTX-D to the Av solution was repeated in total of 8 times. Finally the reaction was quenched using 50 μg/mL of Biotin, (Sigma Aldrich, #B4501-1G), and after incubating for 5 min, 1 mL of the sample was precipitated with 50 μL of Protein G agarose. Confirmation of successful conjugation was demonstrated using SDS-PAGE with a new band assigned to the conjugate appearing at 260 kDa and HPLC (column: X Bridge C8, 3.5 μm 3.0×100 mm, detection wavelength=243 nm, 10 μL injections and run gradient: 5-80% ACN/H₂O, 0.1% TFA for 15 min Rt (min)=1.40 biotin, 5.83 (Target Ab-DTX-D conjugate); 7.24 (unreacted Ab), 9.84 (unreacted DTX-D).

### Example 30

Preparation of an Antibody Activated with an Azide Joining Unit

A solution of coupling buffer (0.1 M sodium acetate+0.15 M NaCl, pH 5.5) was prepared and used to make up stock solutions for the following reaction. Solid sodium meta-periodate (2.1 mg) was dissolved in coupling buffer (0.5 mL) and then was added to a solution of Her2 mAb* (25 μg) also diluted in coupling buffer (0.5 mL). The reaction mixture was incubated at room temperature (RT) in the dark for 45 min. Unreacted material was removed by centrifugal filter units (MW cut off 50 kDa). To a portion of the oxidised mAb solution (0.3 mL) was added a stock solution of a azide containing joining unit (JU) (NH₂—O—C₄H₈—NH—(PEG)₁₂-N₃^{¥}, 0.2 mL; 1 mg/mL in PBS), followed by aniline (5 μL). The reaction was mixed and left for 24 h at RT. After this time the mAb-JU conjugate was separated from unreacted material by centrifugal filter units.

^{¥} In a similar manner other joining units could also be installed onto the antibody, e.g. NH₂—O—C₄H₈—NH—(PEG)₁₂-benzylazide, NH₂—O—C₄H₈—NH—(PEG)₁₂-DBCO and NH₂—O—C₄H₈—NH—(PEG)₁₂-maleimide.
In this example Her2 mAb is utilised however, in a similar fashion other antibodies could also be utilised. In addition to utilising other activating chemistry's e.g. partial reduction of dithiane groups within the antibody followed by capture with maleimide containing joining units

### Example 31

Conjugation of the Activated Antibody with a Drug Loaded Dendrimer

To a solution of the azide activated mAb-JU from Example 30 above could be added a solution of a drug loaded dendrimer suitably functionalised with a reactive alkyne, such as DBCO. The reaction could be monitored for completion using HPLC and the desired product could be isolated by either SEC chromatography or prep HPLC using standard protocols.

^{¥} In a similar manner other dendrimer activating units could also be installed onto the unique point of attachment in the dendrimer, e.g. azide and maleimide.

### Example 32

### Water Solubility Study on Drug Loaded Dendrimers:

Protocol: To 30 mg of dendrimer (freeze-dried from water) was added 100 μL of deionised water. After mixing for 10 minutes, additional aliquots of water (10-30 μL per addition) were added with vortexing and incubation for 10 mins until full dissolution was obtained. This amount is represented in Table 1 as the water solubility of the dendrimer. The equivalent drug solubility is determined by multiplying the % drug loading/100 and is represented in Table 1 (column 3) as Equivalent drug solubility on dendrimer. Finally, the fold increase is obtained by dividing the Equivalent drug solubility on dendrimer by the solubility of the drug and is represented in Table 1 (column 4).

TABLE 1

| | | 3 | |
|---|---|---|---|
| | 2 | Equivalent | 4 |
| | Water | drug | Fold |
| | solubility of | solubility on | increase |
| 1 | dendrimer | dendrimer | in drug |
| Example | (mg/mL) | (mg/mL) | solubility |
| 1 (b) * | 186 | 24 | 4800 |
| 2 (b) * | 57 | 14 | 2800 |
| 3 (b) * | 89 | 23 | 5600 |
| 4 (c) * | 109 | 22 | 4400 |
| 5 (b) * | 214 | 75 | 4000 |
| 6 (b) * | 100 | 32 | 6400 |
| 7 (b) * | 91 | 25 | 5000 |
| 8 (c) * | 131 | 41 | 8200 |
| 9 (b) * | 63 | 20 | 4000 |
| 10 (b) * | 138 | 43 | 8600 |
| 12 (b) * | 15 | 3 | 10000 |
| 14 (c) * | 183 | 57 | 11400 |
| 15 (c) * | 180 | 45 | 9000 |
| 16 * | 205 | 59 | 11800 |
| 17 * | 373 | 67 | 13400 |
| 19 * | 477 | 109 | 21900 |
| 20 (b) ¥ | >75 | 11.5 | 482 |
| 21 ¥ | >81 | 14.8 | 618 |
| 22 (b) ¥ | >89 | 14.7 | 610 |
| 23 ¥ | >125 | 26.6 | 1109 |

* drug = docetaxel.
The solubility of docetaxel and in water is 5 μg/mL
¥ drug = testosterone:
The solubility of testosterone in water is 24 μg/mL.

### Example 33

### Plasma Stability Study on Dendrimers:

Protocol: To 0.5 mL of mouse plasma was added 0.1 mL of dendrimer solution (2 mg/mL, drug equivalent in saline). The mixtures were vortexed (30 s) then incubated at 37° C. At various timepoints (0.5, 2.5, 4.5, 22 hours) 0.1 mL aliquots were removed and added to 0.2 mL ACN. The resulting mixtures were vortexed (30 s), centrifuged (10 min, 4° C.) filtered and analysed by HPLC (C8, 3.9×150 mm, 5 μm, wavelength=243 nm, 10 μL injections, gradient: 40-80% ACN/H₂O (1-7 min), 80% ACN (7-9 min), 80-40% ACN (9-11 min), 40% ACN (11-15 min), 10 mM ammonium formate, pH 7.40) which when compared against a standard (2 mg//mL) provided the concentration of free docetaxel in the sample.

TABLE 2
Docetaxel release in plasma. Results are
shown as a percentage of total docetaxel.

| Time/Example | | | | |
|---|---|---|---|---|
| Compound | 0.5 | 2.5 | 4.5 | 22 |
| Exp 3 (b) | 8.5 | 32.5 | 52.5 | 73 |
| Exp 10 (b) | 10 | 21 | 28.5 | 75 |
| Exp 7 (b) | 20.5 | 32 | 32.5 | 71.5 |
| Exp 14 (c) | 4 | 9 | 16 | 70 |
| Exp 8 (c) | 4.5 | 13.5 | 17.5 | 43 |
| Exp 6 (b) | 7.5 | 9 | 13 | 23.5 |
| Exp 4 (c) | 1.5 | 10 | 18.5 | 17.5 |
| Exp 2 (b) | 5 | 8 | 11.5 | 15.5 |
| Exp 1 (b) | 0 | 3 | 7.5 | 14.5 |
| Exp 15 (c) | 0 | 5 | 8 | 45 |
| Exp 5 (b) | 0 | 0 | 0 | 4 |
| Exp 9 (b) | 0.5 | 1.5 | 1 | 1 |
| Exp 16 | 0 | 0 | 0 | 0 |
| Exp 17 | 0 | 0 | 0 | 1 |

### Example 34

### Cell Growth Inhibition Studies SRB Assay

Cell growth inhibition was determined using the Sulforhodamine B (SRB) assay [Voigt W. “Sulforhodamine B assay and chemosensitivity” Methods Mol. Med. 2005, 110, 39-48.] against various cancer cell lines after 72 hours with each experiment run in duplicate. GI₅₀ is the concentration required to inhibit total cell growth by 50%, as per NCI standard protocols.

All solutions were prepared in saline (except docetaxel which was made in ethanol). All solutions were stored at −20° C. All values were based on the equivalent drug loading. The results shown in Table 3 are the average of experiments run in duplicate in nanomolar range.

TABLE 3
Growth Inhibition Studies.

| | GI₅₀ Values (nM) | | | | | | | | | | | |
|---|---|---|---|---|---|---|---|---|---|---|---|---|
| | | Exp | Exp | Exp | Exp | Exp | Exp | Exp | Exp | Exp | Exp | Exp |
| Cell line | Docetaxel | 1 (b) | 3 (b) | 4 (c) | 5 (b) | 13 (b) | 2 (b) | 6 (b) | 7 (b) | 8 (c) | 9 (b) | 10 (b) |
| PC-3 (Prostate) | 2.5 | 17 | 4.5 | 21.5 | 160 | 288 | 109.5 | 10.5 | 6.5 | 9.5 | 617.5 | 9.5 |
| DU145 (Prostate) | 2.5 | 11.5 | 4 | 12 | 148 | 99 | | | | | | |
| HCT116 (Colon) | 0.7 | 8.5 | 1 | 9 | 85.5 | 30.5 | | | | | | |
| ES2 (Ovarian) | 5 | 16.5 | 4 | 8.6 | 115.5 | 48 | 115.5 | 12.5 | 8 | 12 | 888 | 10.5 |
| HT29 (Colon) | 1.5 | 12.5 | 2 | 9.5 | 97.5 | 117 | | | | | | |
| H460 (Lung) | 1.5 | 13 | 8 | 11 | 106 | 127 | 73 | 11 | 4.5 | 7 | 365 | 6.5 |
| A549 (Lung) | 3.5 | 13 | 3.5 | 8.5 | 56.5 | 73 | | | | | | |
| MDA-MB-231 (Breast) | 3.5 | 11.5 | 0.5 | 6.5 | 50.5 | 50.5 | | | | | | |
| A2058 (Melanoma) | 2 | 9.5 | 2 | 8 | 71.5 | 100.5 | | | | | | |
| MCAS (Ovarian) | 7 | 29 | 7 | 20 | 252.5 | 117 | | | | | | |

### Example 35

### Half Maximal Inhibitory Concentration (IC₅₀) Using the MTT Assay

The IC₅₀ using the MTT assay [Wilson, Anne P. (2000). “Chapter 7: Cytotoxicity and viability”. In Masters, John R. W. Animal Cell Culture: A Practical Approach. Vol. 1 (3rd ed.). Oxford: Oxford University Press] was determined against various cancer cell lines after 72 hours. The results are shown in Table 4.

TABLE 4
Half Maximal Inhibitory Concentration Studies (IC₅₀).

| | IC₅₀ Values (nM) | | | | |
|---|---|---|---|---|---|
| Cell line | Exp 14 (c) | Exp 15 (c) | Exp 17 | Exp 18 | Exp 19 |
| A549 | 1.5 | 8.1 | 159.7 | 20.3 | 7.7 |
| H460 | 4.3 | 31.8 | 603.3 | 7.5 | 23.7 |
| HCT-116 | 2.6 | 7.2 | 215.7 | 2.9 | 6.5 |
| HT-29 | 0.5 | 5.7 | 85 | 1.8 | 5.9 |
| A2780 | 4.6 | 13.6 | 291 | 5.7 | 6.3 |
| MCF-7 | 0.5 | 8.3 | 93.7 | 3.3 | 6.3 |
| DU-145 | 7.3 | 29.5 | 290 | 11.6 | 15.5 |
| PC-3 | 3.8 | 11.8 | 358.7 | 5.9 | 7.4 |

### Example 36

### Maximum Tolerated Dose (MTD) Study

Groups of female Balb/c mice were administered an intravenous injection of dendrimer (0.1 ml/10 g body weight) or docetaxel (0.05 ml/10 g body weight) once weekly for 3 weeks (day 1, 8 and 15). Mice were weighed daily and watched for signs of toxicity. Animals were monitored for up to 10 days following the final drug dose. Any mice exceeding ethical endpoints (≥20% body weight loss, poor general health) were immediately sacrificed and observations were noted. The results shown in Table 5 demonstrate that drug conjugated to the dendrimer increases the tolerated dose. More than twice the dose of docetaxel could be safely administered using drug dendrimer construct compared to docetaxel alone.

TABLE 5
Drug doses tested and maximum tolerated dose identified

| | Doses tested | Tolerated dose |
|---|---|---|
| | (mg/kg | (mg/kg |
| | docetaxel | docetaxel |
| Drug | equivalents) | equivalents) |
| Docetaxel | 15, 20, 25, 30 | 15 |
| Example 3 (b) | 15, 20, 23, 25, 30 | 20 |
| Example 8 (c) | 15, 20, 25, 30, 32, 35 | 32 |
| Example 4 (c) | 20, 25, 30 | 20 |

### Example 37

### Xenograft MDA-MB-231 Efficacy Study

Female Balb/c nude mice (Age 7 weeks) were inoculated subcutaneously on the flank with 3.5×106 MDA-MB-231 cells in PBS:Matrigel (1:1). Thirteen days later 50 mice with similar sized tumours (˜110 mm³) were randomised into 5 groups. Each treatment group was administered one of the following doses: saline; docetaxel (15 mg/kg); Exp. 3 (b) (20 mg/kg); Exp. 8 (c) (32 mg/kg). All treatments were administered intravenously once weekly for three weeks (day 1, 8 and 15) at 0.1 mL/10 g body weight except docetaxel which was given at 0.05 mL/10 g body weight. The experiment was ended on day 120 or earlier if an ethical endpoint was met. Results shown in Table 6 show that the dendrimer constructs were more effective in suppressing tumour growth for longer.

TABLE 6
Xenograft efficacy study showing mean tumour volume mm³ over time

| | Mean tumour Volumne mm³ (sd) | | | |
|---|---|---|---|---|
| Day | Vehicle | Docetaxel | Exp 3 (b) | Exp 8 (c) |
| 1 | 112.35 (6.31), | 111.94 (6.41), | 111.74 (6.65), | 111.73 (6.41), |
| | n = 10 | n = 10 | n = 10 | n = 10 |
| 9 | 426.55 (24.11), | 135.57 (18.85), | 84.02 (6.33), | 108.86 (9.31), |
| | n = 10 | n = 10 | n = 10 | n = 10 |
| 19 | 1337.61 (18.4), | 49.92 (11.61), | 28.26 (1.91), | 30.59 (1.64), |
| | n = 4 | n = 10 | n = 10 | n = 10 |
| 29 | ** | 18.81 (2.09), | 10.46 (0.5), | 11.58 (1.2), |
| | | n = 10 | n = 8 | n = 9 |
| 40 | | 10.75 (1.95), | 5.92 (1.31), | 5.75 (0.92), |
| | | n = 10 | n = 5 | n = 8 |
| 61 | | 95.94 (33.08), | 4 (0), | 4 (0), |
| | | n = 10 | n = 4 | n = 8 |
| 81 | | 478.67 (169.27), | 0.5 (0), | 0.5 (0), |
| | | n = 7 | n = 4 | n = 8 |
| 100 | | 974.83 (302.59), | 0.5 (0), | 1.67 (0.74), |
| | | n = 3 | n = 4 | n = 6 |
| 120 | | ** | 0.37 (0.12), | 16.2 (10.24), |
| | | | n = 4 | n = 6 |

** No data due to ethical endpoint reached.
n = number of animals per dosing group

### Example 38

### Xenograft MDA-MB-231 Toxicity Study

A total of twenty Female Balb/c nude mice (Age 7 weeks) were prepared with subcutaneous tumours as outlined above. The 20 mice were randomised into 5 groups of four mice (mean tumour volume ˜90 mm³). Animals were eye bled in the morning for baseline blood cell counts and then drug dosing commenced later that day (day 1). Drug dosing was performed on days 1, 8 and 15 at the previously determined MTD doses: docetaxel (15 mg/kg); Exp. 3 (b) (20 mg/kg); Exp. 8 (c) (32 mg/kg); Exp. 4 (b) (20 mg/kg) . . . . A second eye bleed was performed on day 11 (Table 7 A-C). Mice were killed one day following the final drug dose (day 16). Histology weights of tissues at day 16 are shown in Table 8.

TABLE 7 A
White Blood Cell analysis at days 1 and 11.
Mean WBC (sd) × 10⁹ cells/L

| | PBS | docetaxel | Exp. 3 (b) | Exp. 8 (c) | Exp. 4 (b) |
|---|---|---|---|---|---|
| Day 1 | 5.76 (0.31) | 5.79 (1.01) | 5.79 (1.53) | 6.59 (0.62) | 4.95 (2.25) |
| Day 11 | 8.57 (1.94) | 3.99 (0.93) | 3.99 (0.29) | 4.27 (0.35) | 5.37 (1.72) |

Table 7 B
Results of Neutrophil Analysis at days 1 and 11.
Mean Neutrophils (sd) × 10⁹ cells/L

| | PBS | docetaxel | Exp. 3 (b) | Exp. 8 (c)) | Exp. 4 (b) |
|---|---|---|---|---|---|
| Day 1 | 1.53 (1.12) | 0.86 (0.26) | 1.01 (0.53) | 0.93 (0.51) | 1.07 (0.57) |
| Day 11 | 2.84 (0.62) | 0.85 (0.12) | 1.84 (0.18) | 1.76 (0.15) | 1.27 (0.64) |

TABLE 7 C
Results of Lymphocyte analysis at days 1 and 11.
Mean Lymphocytes (sd) × 10⁹ cells/L

| | PBS | docetaxel | Exp. 3 (b) | Exp. 8 (c) | Exp. 4 (b) |
|---|---|---|---|---|---|
| Day 1 | 5.76 (0.31) | 5.79 (1.01) | 5.79 (1.53) | 6.59 (0.62) | 4.95 (2.25) |
| Day 11 | 8.57 (1.94) | 3.99 (0.93) | 3.99 (0.29) | 4.27 (0.35) | 5.37 (1.72) |

TABLE 8
Organ Weights at Completion of Toxicity Experiment.

| | PBS | Docetaxel | Exp. 3 (b) | Exp. 8 (c) | Exp. 4 (b) |
|---|---|---|---|---|---|
| Mean Tumour | 0.832 | 0.048 | 0.020 | 0.033 | 0.079 |
| Weights (g) | (0.277) | (0.010) | (0.008) | (0.011) | (0.048) |
| (sd) | | | | | |
| Mean Spleen | 0.149 | 0.068 | 0.077 | 0.092 | 0.087 |
| Weights (g) | (0.022) | (0.003) | (0.011) | (0.019) | (0.027) |
| (sd) | | | | | |
| Mean Liver | 0.838 | 0.793 | 0.763 | 0.780 | 0.762 |
| Weights (g) | (0.058) | (0.087) | (0.090) | (0.103) | (0.096) |
| (sd) | | | | | |

### Example 39

### Pharmacokinetic Analysis

The plasma half-lives of tritium labelled docetaxel and the construct from Experiment 8 (c) (prepared using tritium labelled docetaxel) after IV administration into rats were determined (Kaminskas, L. M., Boyd, B. J., Karellas, P., Krippner, G. Y., Lessene, R., Kelly, B and Porter, C. J. H. “The Impact of Molecular Weight and PEG Chain Length on the Systemic Pharmacokinetics of PEGylated Poly-L-Lysine Dendrimers” Molecular Pharm. 2008, 5, 449-463). Results showed docetaxel was cleared from plasma with a half-life of <1 hour as expected whilst Exp 8 (c) construct displayed reduced plasma clearance with a half-life of approximately 30 hour.

### Example 40: Synthesis of Linker-Cabazitaxel

### a) Diglycolic Acid (DGA)-Cabazitaxel

To a solution of Cabazitaxel (2.00 g, 2.39 mmol) in dichloromethane (30 mL, 15 vol.) was added diglycolic anhydride (320.70 mg, 2.62 mmol, 1.1 eq., 95% purity). After stirring for 5 min., triethylamine (500 μL, 3.59 mmol, 1.5 eq.) was added. The reaction mixture was stirred at room temperature for 1.5 h. LC-MS analysis (eluent: 40-80% acetonitrile in water with 0.1% 10 mM ammonium formate buffer) showed presence of less than 1% starting material. The reaction mixture was diluted with 30 mL of DCM and then washed twice with sodium chloride (5%) and sodium phosphate (1%) buffer at pH=3 (30 mL). During the first wash, the pH rose to 6.0, 1M aq. HCl (2.0 mL) was added to readjust the pH at 3.0. Layers separated. DCM extract was dried over MgSO₄ (3.2 g) and filtered through glass sintered funnel. Funnel washed two times with 5 mL (10 mL) DCM. The filtrate was evaporated to give white solid. Yield=2.03 g, 88.5% ¹H NMR: DMSO-d₆. δ (ppm): 0.97 (s, 3H), 0.99 (s, 3H), 1.38 (s, 9H), 1.46-1.60 (m, 5H), 1.77-1.85 (m, 4H), 2.23 (s, 3H), 2.62-2.75 (m, 1H), 3.22 (s, 3H), 3.29 (s, 3H), 3.59 (d, J=6 Hz, 1H), 3.76 (dd, J=6 Hz and 12 Hz, 1H), 4.02 (s, 2H), 4.14 (s, 2H), 4.31 (d, J=18 Hz, 1H), 4.40 (d, J=15 Hz, 1H), 4.51 (s, 1H), 4.71 (s, 1H), 4.96 (d, J=9 Hz, 1H), 5.06 (t, J=9 Hz, 1H), 5.17 (d, J=6 Hz, 1H), 5.38 (d, J=9 Hz, 1H), 5.82 (t, J=9 Hz, 1H), 7.19 (t, J=9 Hz, 1H), 7.35-7.46 (m, 4H), 7.64-7.77 (m, 3H), 7.88 (d, J=9 Hz, 1H), 7.98 (d, J=6 Hz, 2H). LC-MS: C8 XBridge 3.0×100 mm, 120 A, 3.5 μm. 40-80% ACN/H₂O (1-7 min), 80% ACN (7-9 min), 80-40% ACN (9-11 min), 40% ACN (11-15 min), 0.1% 10 mM ammonium formate Rf (min)=5.76. ESI (+ve) observed [M+OH]+=969. Calculated for C₄₉H₆₁NO₁₈=952.02 Da. In process analysis: 25 μl aliquot was diluted with 1 ml acetonitrile. Isolated material: Approximately 1.0 mg/ml solution in acetonitrile.

### b) Thiodiglycolic Acid (TDA)-Cabazitaxel

Prepared using Procedure in Example 40a above, using CTX (400 mg, 479 μmol) and thiodiglycolic anhydride (95 mg, 718 μmol) as the linker. The product was isolated as a white powder. LCMS (C8, gradient: 40-90% ACN/H₂O (1-7 min), 90% ACN (7-9 min), 90-40% ACN (9-11 min), 40% ACN (11-15 min), 0.1% Formic acid) Rf (min)=7.98. ESI (+ve) observed [M]⁺=968.20. Calculated for C₄₉H₆₁NO₁₇S=968.07 Da.

### c) Methyliminodiacetic Acid (MIDA)-Cabazitaxel

Prepared using Procedure in Example 1a above, using CTX (400 mg, 479 μmol) and MIDA anhydride (93 mg, 718 μmol) as the linker. The product was isolated as a white powder. LCMS (C8, gradient: phobic formic 40-90% ACN/H₂O (1-7 min), 90% ACN (7-9 min), 90-40% ACN (9-11 min), 40% ACN (11-15 min), 0.1% Formic acid) Rf (min)=5.60. ESI (+ve) observed [M]⁺=965.5. Calculated for C₅₀H₆₄N₂O₁₇=965.05 Da.

### Example 41: Synthesis of Cabazitaxel-Containing Dendrimers

a) BHALys[Lys]₃₂[α-DGA-Cabazitaxel]_{32†}[ε-PEG_{˜2100}]_{32‡} (SPL9048)

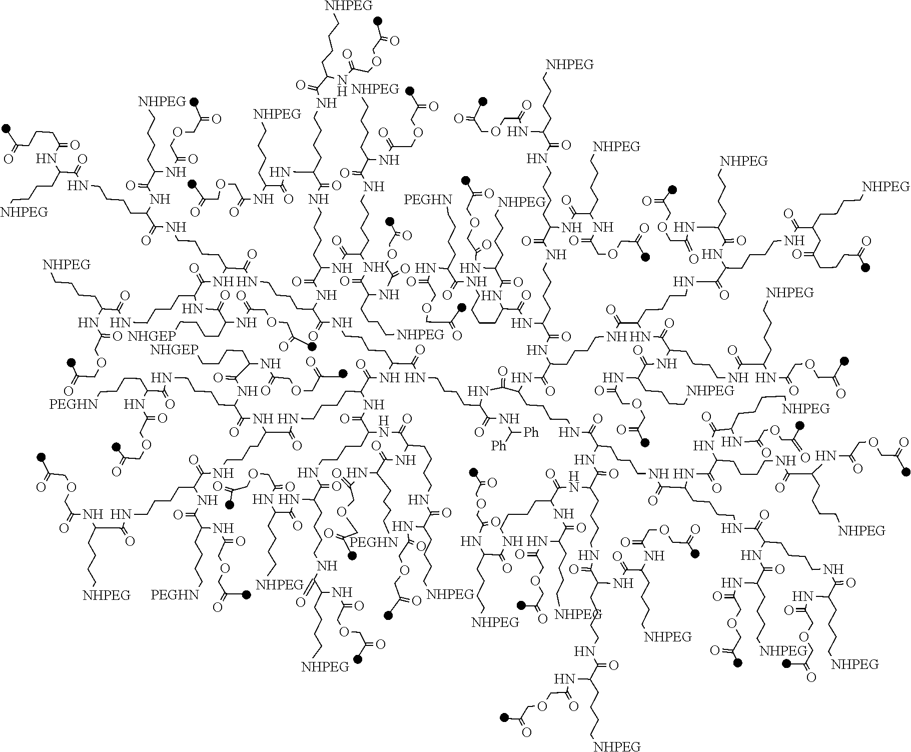

PEG represents-C(O) CH₂-PEG_{˜2100} in which PEG_{˜2100} represents a methoxy-terminated PEG group having approximate average molecular weight of 2100 Daltons (e.g. an average molecular weight in the range of about 1900 to 2300); and • represents a residue of Cabazitaxel.

Note: 32† relates to the theoretical number of a surface amino groups on the dendrimer available for substitution with DGA-Cabazitaxel. The actual mean number of DGA-Cabazitaxel groups attached to BHALys[Lys]₃₂ was determined experimentally by ¹H NMR using 3,4,5-Trichloro pyridine as an internal standard.

Note: 32‡ relates to the theoretical number of & surface amino groups on the dendrimer available for substitution with PEG_{˜2100}. The actual mean number of PEG_{˜2100} groups attached to the BHALys[Lys]₃₂ was determined experimentally by ¹H NMR.

To a solution of DGA-Cabazitaxel (2.020 g, 2.12 mmol, 1.2 eq/NH₂) in DMF (20 mL, 4.8 Vol.) was added solid PyBOP (1.15 g, 2.21 mmol, 1.25 eq/NH₂). After 5 min stirring at rt, solid BHALys[Lys]₃₂[α-NH₂TFA]₃₂[ε-PEG 2100]_{32‡} (4.19 g, 55.25 μmol) was added. DMF (3 mL) was used to rinse residual solids from vials. Suspension was stirred at RT and mixture became homogeneous within 15 min. NMM (0.97 mL, 8.84 mmol, 5 eq/NH₂) was added. A pale yellow solution formed, and was stirred at rt for 24 h. The solution was diluted with ACN (24 mL) and filtered through 0.45 μm filter. BHALys[Lys]₃₂[α-DGA-Cabazitaxel]_{32 †}[ε-PEG 2100]_{32‡} was isolated by Ultrafiltration in acetonitrile (15 Diafiltration volumes) using a 0.1 m² 10 kda Pelicon 3 regenerated cellulose membrane. Retentate solution was concentrated in vacuo to give a yellow gum which was dissolved in THF (60 mL) and was filtered through 0.45 μm filter. The filtrate was concentrated in vacuo to obtain a gum. The yellow gum was dissolved in THF (27.5 ml, 4.9 vol based off theoretical yield of 5.6 g BHALys[Lys]₃₂[α-DGA-Cabazitaxel]_{32†}[ε-PEG 2100]_{32‡}) and was added via dropping funnel over 1 h to vigorously stirred MTBE (110 mL, 20 vol), cooled in an ice bath and under N₂. A fine white suspension formed with some clumps and some material stuck to flask walls. Once addition was complete, the suspension was stirred on ice for a further 60 min. The flask was then removed from the ice bath and allowed to warm to room temperature with stirring. Solids on flask walls were mostly dislodged using a spatula and the solid was collected by filtration over a P3 sintered funnel. Clumps were broken using a metal spatula and the filtered solid was washed with MTBE (2×28 mL). The wet cake was transferred to a vial and residual MTBE removed under vacuum at room temperature to afford a fine white powder; 5.35 g, 94.9%. ¹H NMR: CD₃OD-d₄. δ (ppm): 1.13-2.73 (m, 1225H), 3.23-3.30 (m, 57H), 3.37 (s, 99H), 3.39-3.97 (m, 5720H), 4.04-4.50 (m, 114H), 5.003 (br s, 27H), 5.39-5.6.15 (m, 108H), 7.28-8.10 (m, 334H). 3,4,5-Trichloro pyridine was used as internal standard and loading was calculated by comparing Cabazitaxel aromatic signals with 3,4,5-trichloropyridine signals. Theoretical molecular weight of conjugate: 102 kDa. ¹H NMR suggests 29.8 CTX/dendrimer. Actual molecular weight is approximately 100 kDa (24.9% CTX by weight). HPLC (C8 Phenomenex Kinetex 2.1×75 mm, 100 A, 2.6 μm. 5-45-90% ACN (with 0.1% TFA) in water (with 0.1% TFA) gradient: 5% (0-1 min), 5-45% ACN/H₂O (1-2 min), 45% ACN (2-10 min), 45-90% (10-14 min), 90% (14-18 min), 90-5% ACN (18-18.1 min), 5% ACN (18.1-20 min) Rf (min)=14.03. In process analysis: 5 μL aliquot was diluted with 1 mL acetonitrile. Isolated material: Approximately 3.0 mg/ml solution in acetonitrile.

b) BHALys[Lys]₃₂[α-TDA-Cabazitaxel]_{32†}[ε-PEG_{˜2100}]_{32‡} (SPL9005)
Prepared as in a) above using TDA-Cabazitaxel (463 mg, 479 μmol, 2.0 eq/NH₂), PyBOP (249 mg, 479 μmol, 2.0 eq/NH₂), BHALys[Lys]₃₂[α-NH₂TFA]₃₂[ε-PEG_{˜2100}]_{32‡} (578 mg, 7.48 μmol) and NMM (158 μL, 1.44 mmol, 6 eq/NH₂).
Yield: 740 mg, 95.1%, fine white powder

¹H NMR: CD₃OD-d₄. δ (ppm): 1.13-2.77 (m, 1166H), 3.13-3.30 (m, 128H), 3.37 (s, 126H), 3.38-3.44 (m, 85H), 3.48-3.76 (m, 5510H), 3.78-4.50 (m, 284H), 5.02 (br s, 34H), 5.31-5.60 (m, 81H), 6.14 (br s, 24H), 7.27-7.69 (m, 233H), 8.10 (br s, 58H). 2,4,5-Trichloropyrimidine was used as internal standard and loading was calculated by comparing Cabazitaxel aromatic signals with 2,4,5-trichloropyrimidine signals. Theoretical molecular weight of conjugate: 104 kDa (25.7% CTX). ¹H NMR suggests 32 CTX/dendrimer. Actual molecular weight is approximately 104 kDa. 25.9% CTX by weight as determined by NMR.

HPLC (C8 XBridge 3×100 mm, 120 A, 3.5 μm. 5-80% ACN (with 0.1% ammonium formate) in water (with 0.1% ammonium formate): Rf (min)=8.70
c) BHALys[Lys]₃₂[α-MIDA-Cabazitaxel]_{32†} [ε-PEG_{˜2100}]_{32‡} (SPL9006)
Prepared as in a) above using MIDA-Cabazitaxel (440 mg, 456 μmol, 2.0 eq/NH₂), PyBOP (237 mg, 456 μmol, 2.0 eq/NH₂), BHALys[Lys]₃₂[α-NH₂TFA]₃₂[ε-PEG 2100]_{32‡} (550 mg, 7.12 μmol) and NMM (150 μL, 1.37 mmol, 6 eq/NH₂).
Yield: 708 mg, 95.7% fine white powder

¹H NMR: CD₃OD-d₄. δ (ppm): 1.13-2.74 (m, 1235H), 3.13-3.28 (m, 145H), 3.37 (s, 126H), 3.38-3.42 (m, 100H), 3.51-3.78 (m, 5510H), 3.86-4.37 (m, 260H), 5.02 (br s, 43H), 5.35-5.61 (m, 95H), 6.14 (br s, 33H), 7.27-7.91 (m, 250H), 8.10 (br s, 64H). 2,4,5-Trichloropyrimidine was used as internal standard and loading was calculated by comparing Cabazitaxel aromatic signals with 2,4,5-trichloropyrimidine signals. Theoretical molecular weight of conjugate: 104 kDa (25.8% CTX). ¹H NMR suggests 29 CTX/dendrimer. Actual molecular weight is approximately 101 kDa. 23.3% CTX by weight as determined by NMR

HPLC (C8 XBridge 3×100 mm, 120 A, 3.5 μm. 5-80% ACN (with 0.1% ammonium formate) in water (with 0.1% ammonium formate): Rf (min)=8.61
d) BHALys[Lys]₃₂[α-DGA-Cabazitaxel]_{32†} [ε-PEG_{˜1100}]_{32‡} (SPL9049)
Prepared as in a) above using DGA-Cabazitaxel (548 mg, 575 μmol, 1.6 eq/NH₂), PyBOP (299 mg, 575 μmol, 1.6 eq/NH₂), BHALys[Lys]₃₂[α-NH₂TFA]₃₂[ε-PEG_{˜1100}]_{32‡} (540 mg, 11.2 μmol) and NMM (237 μL, 2.16 mmol, 6 eq/NH₂).
Yield: 844 mg, >100% white powder

¹H NMR: CD₃OD-d₄. δ (ppm): 1.15-2.73 (m, 1260H), 3.18-3.28 (m, 64H), 3.35 (s, 89H), 3.39-3.45 (m, 63H), 3.51-3.73 (m, 2643H), 3.76-4.55 (m, 280H), 5.02 (br s, 28H), 5.39-5.60 (m, 82H), 6.16 (br s, 26H), 7.29-7.68 (m, 245H), 8.11-8.13 (m, 59H). 3,4,5-Trichloropyridine was used as internal standard and loading was calculated by comparing Cabazitaxel aromatic signals with 3,4,5-trichloropyridine signals. Theoretical molecular weight of conjugate: 70 kDa (38.3% CTX). ¹H NMR suggests 27 CTX/dendrimer. Actual molecular weight is approximately 65 kDa. 34.7% CTX by weight as determined by NMR

HPLC (C8 XBridge 3×100 mm, 120 A, 3.5 μm. 5-80% ACN (with 0.1% ammonium formate) in water (with 0.1% ammonium formate): Rf (min)=10.1
e) N₃-PEG₂₄-CO(NPN)[Lys]₃₂[α-TDA-Cabazitaxel]_{32†}[ε-PEG_{˜1100}]_{32‡} (SPL8996)

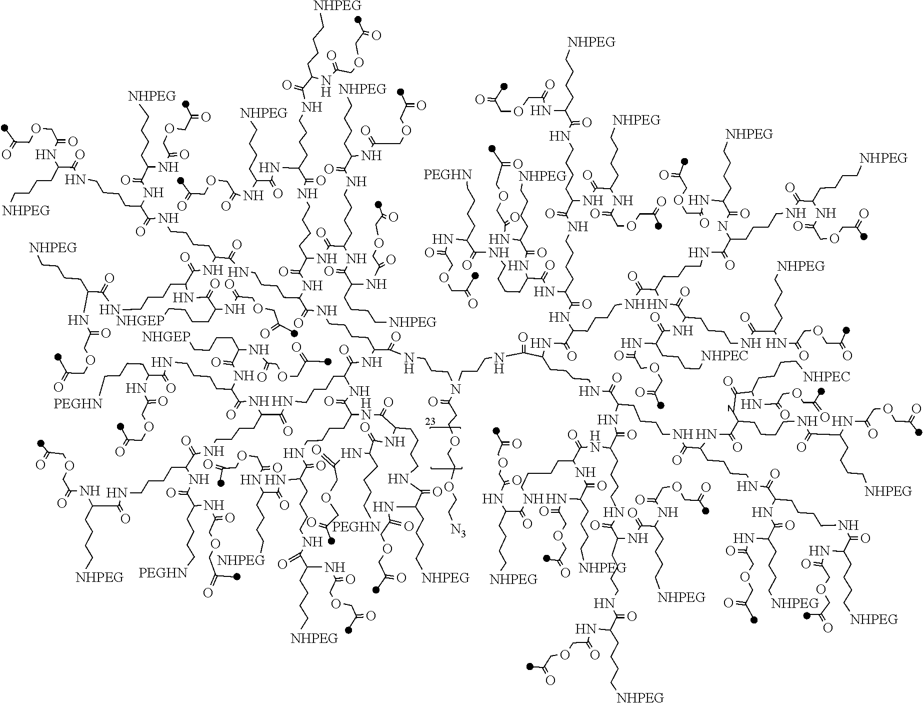

PEG represents-C(O) CH₂-PEG_{˜1100} in which PEG_{˜1100} represents a methoxy-terminated PEG group having approximate average molecular weight of 1100 Daltons; and
represents a residue of Cabazitaxel.
   Note: 32† relates to the theoretical number of a surface amino groups on the dendrimer available for substitution with TDA-Cabazitaxel. The actual mean number of TDA-Cabazitaxel groups attached was determined experimentally by ¹H NMR using 3,4,5-Trichloro pyridine as an internal standard.
   Note: 32‡ relates to the theoretical number of ε surface amino groups on the dendrimer available for substitution with PEG_{˜1100}. The actual mean number of PEG_{˜1100} groups attached was determined experimentally by ¹H NMR.

### i) Preparation of Azido-PEG₂₄-Triamino Core Group

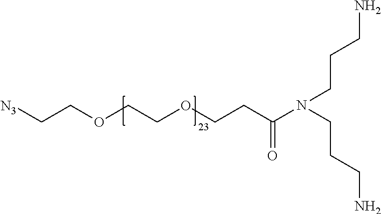

1,9-bis-Boc-1,5,9-triazanonane (a di-protected triamino compound) was reacted with azido-PEG₂₄-acid to form the above azido-PEG₂₄-triamino core group.
ii) Preparation of N₃-PEG₂₄-CO(NPN) [Lys]₃₂[α-NH₂TFA]_{32‡} [ε-PEG_{˜1100}]₃₂;
The Boc groups present on the amino-propyl units were then deprotected to make available the two nitrogen atoms for reaction with the lysine building units. The amine groups were then reacted with amine-protected lysines to form the first generation of the dendrimer as outlined in WO2008/017125 (see page 61, step vi). Conversion into N₃-PEG₂₄-CO(NPN) [Lys]₃₂[α-NH₂TFA]_{32†} [ε-PEG_{˜1100}]_{32‡} may be achieved by following an analogous synthetic process to that described in Kaminskas et al., J Control. Release (2011) doi 10.1016/j.jconrel.2011.02.005 for the preparation of BHALys[Lys]₃₂[α-NH₂. TFA]₃₂[ε-PEG₁₁₀₀]₃₂.
iii) Preparation of N₃-PEG₂₄-CO(NPN) [Lys]₃₂[α-TDA-Cabazitaxel]_{32†}[ε-PEG_{˜1100}]_{32‡}
The cabazitaxel-containing dendrimer was prepared in an analogous manner to that described in a) above using TDA-cabazitaxel and N₃-PEG₂₄-CO(NPN) [Lys]₃₂[α-TDA-Cabazitaxel]_{32†} [ε-PEG_{˜1100}]_{32‡}.

¹H NMR: CD₃OD-d₄. δ (ppm): 0.70-2.80 (m, 1338H), 3.00-3.20 (m, 62H), 3.32-4.01 (m, 3487H), 4.04-4.58 (m, 105H), 5.03 (br s, 30H), 5.20-5.50 (m, 54H), 5.60 (br s, 27H), 6.65 (br s, 28H), 7.10-8.40 (m, 320H). ¹H NMR suggests approximately 30 CTX/dendrimer (CTX signals between 5.0 and 8.4 ppm). Actual molecular weight approximately 72.9 kDa (34.4% CTX by weight).

### Example 42: Efficacy of Cabazitaxel-Dendrimer Compounds in Breast Cancer Tumour Model in Mice

MDA-MB-231 (human breast carcinoma cell line) mouse xenograft breast cancer model studies were carried out to assess the anti-tumour efficacy properties of the following dendrimers and free cabazitaxel:
SPL8996, N₃-PEG₂₄-CO(NPN) [Lys]₃₂[α-TDA-Cabazitaxel]_{32†} [ε-PEG 1100]_{32‡};
SPL9005, BHALys[Lys]₃₂[α-TDA-Cabazitaxel]_{32†} [ε-PEG 2100]_{32‡};
SPL9006, BHALys[Lys]₃₂[α-MIDA-Cabazitaxel]_{32†} [ε-PEG 2100]_{32‡}
SPL9048, BHALys[Lys]₃₂[α-DGA-Cabazitaxel]_{32†} [ε-PEG 2100]_{32‡}.

References to amounts dosed in mg/kg for the dendrimeric compounds are to the amounts of cabazitaxel that may theoretically be released by the dendrimers.

MDA-MB-231 (human breast carcinoma cell line) mouse xenograft breast cancer model studies were carried out to assess the anti-tumour efficacy properties of SPL8996, SPL9005, SPL9006 and SPL9048 versus free cabazitaxel, in female Balb/c nude mice.

Each of the dendrimers was pre-weighed in glass vials and stored at 20° C. until use, and dissolved in saline immediately prior to dosing. Cabazitaxel was purchased from a commercial supplier.

Female Balb/c nude mice (age 7 weeks) were inoculated subcutaneously on the flank with 3.5×10⁶ MDA-MB-231 cells in PBS:Matrigel (1:1). Mice were weighed and tumours measured twice weekly using electronic callipers. Tumour volume (mm³) was calculated as length (mm)/2×width (mm)².

For the study involving SPL9048, on day 10 after implantation (referred to as Day 1) mice with similar sized tumours (mean tumour volume 90 mm³) were randomised into 4 groups of 10 animals. Treatment groups were saline, cabazitaxel (9 mg/kg), SPL-9048 (9 mg/kg) and SPL-9048 (10 mg/kg). All compounds were given intravenously by tail vein injection on days 1, 8 and 15 at 0.1 ml/10 g body weight except cabazitaxel which was given at 0.05 ml/10 g body weight. Mice received a small dish containing a food supplement (mixed with food dust) daily. The experiment was ended on day 113 or earlier if an ethical endpoint was met.

For the study involving SPL8996, SPL9005 and SPL9006, on day 12 after implantation (referred to as Day 1), mice with similar sized tumours (mean tumour volume 122 mm²) were randomised into 5 groups of 12 animals. Treatment groups were saline, cabazitaxel (10 mg/kg), SPL-8996 (28 mg/kg), SPL-9005 (28 mg/kg) and SPL-9006 (28 mg/kg). All compounds were given intravenously by tail vein injection on days 1, 8 and 22 at 0.1 ml/10 g body weight except SPL-9006 which was given on days 1 and 8 only. Mice received a small dish containing a food supplement (mixed with food dust) daily. The experiment was ended on day 150 or earlier if an ethical endpoint was met.

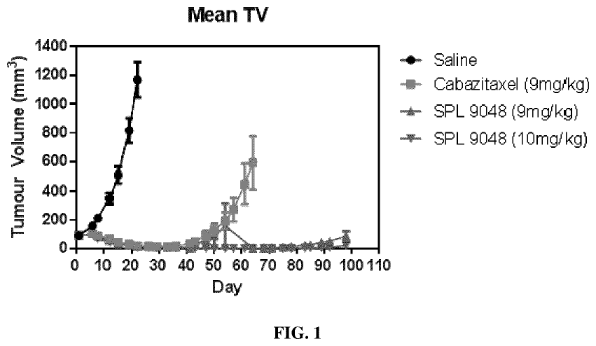

FIG. 1 shows the antitumour efficacy of the SPL-9048 treatments against the MDA-MB-231 tumour xenografts. Tumour volumes were determined twice weekly and were expressed as mean tumour volume (±SEM). Each group initially consisted of 10 mice and graphs are shown until no fewer than 7 animals remained in a group. As shown in FIG. 1, SPL9048 induced complete tumour regression. Tumour regrowth in the cabazitaxel group was evident by day 43 with 9 of 10 tumours reaching an ethical tumour volume endpoint by day 98. Both doses of SPL-9048 significantly extended survival beyond that of cabazitaxel.

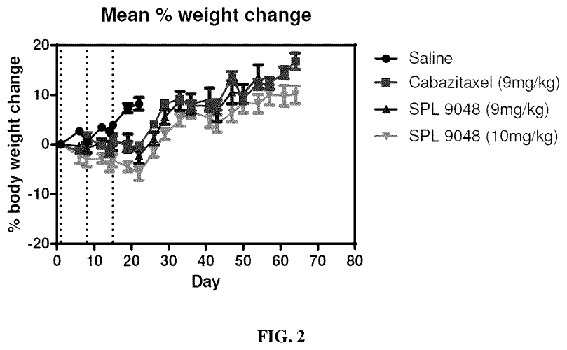

FIG. 2 shows the effect of saline, cabazitaxel, and SPL-9048 on MDA-MB-231 tumour-bearing mouse body weight. Each group initially consisted of 10 mice. Drugs were administered i.v. on days 1, 8 and 15 (indicated by the vertical lines). The data represent the mean percent weight change from baseline (Day 1) for each group; bars SEM. Graphs are shown for each group until fewer than 7 animals remained in each group. As shown in FIG. 2, SPL9048 was overall well tolerated and mean weight loss did not exceed 6% in any group.

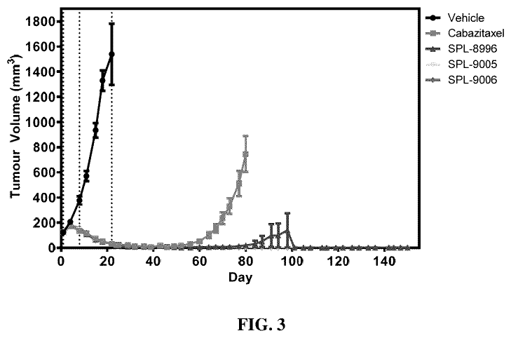

FIG. 3 shows the antitumour efficacy of the SPL-8996, SPL-9005 and SPL-9006 treatments against the MDA-MB-231 tumour xenografts. Tumour volumes were determined two to three times weekly and were expressed as mean tumour volume (±SEM). Each group initially consisted of 12 mice and graphs are shown until no fewer than 9 animals remained in a group. As shown in FIG. 3, all drug treatments initially induced complete tumour regression. Resumption of tumour growth was observed in the cabazitaxel group by day 60. With the exception of one tumour in the SPL-8996 group which began to regrow by day 77, no tumour regrowth was observed in the dendrimer treated groups at the conclusion of the study on day 150. Tumour growth in all drug treated groups was significantly inhibited compared with the vehicle group on day 18 (P<0.00001). Survival of mice in the cabazitaxel treatment group was significantly prolonged vs vehicle group (P<0.00001) while survival in the SPL-8996, SPL-9005 and SPL-9006 groups was significantly prolonged vs cabazitaxel (P=0.0003, 0.0001 and 0.0001 respectively).

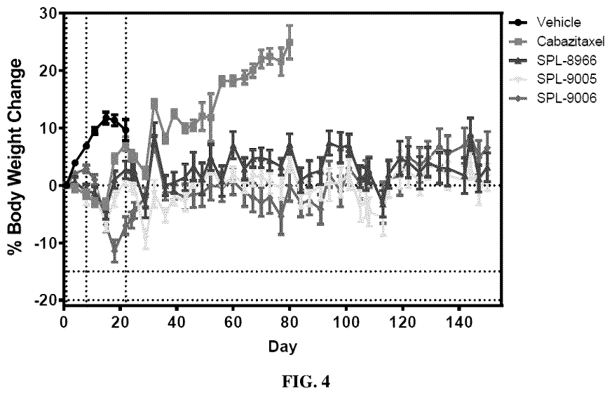

FIG. 4 shows the effect of saline, cabazitaxel, and SPL-8996, SPL-9005 and SPL-9006 on MDA-MB-231 tumour-bearing mouse body weight. Each group initially consisted of 12 mice. Drugs were administered i.v. on days 1, 8 and 22 except SPL-9006 which was given on days 1 and 8 only. The data represent the mean percent weight change from baseline (Day 1) for each group; bars SEM. Graphs are shown for each group until fewer than 7 animals remained in each group.

### Example 43: Toxicity Studies

Toxicity studies in rats were carried out comparing the effects of SPL9048 and free cabazitaxel (Jevtana®).

SPL9048 and Jevtana® were dosed at 1 mg/kg to rats, n-6 (3 males, 3 females). References to amounts dosed in mg/kg for the dendrimeric compound are to the amounts of cabazitaxel that may theoretically be released by the dendrimer.

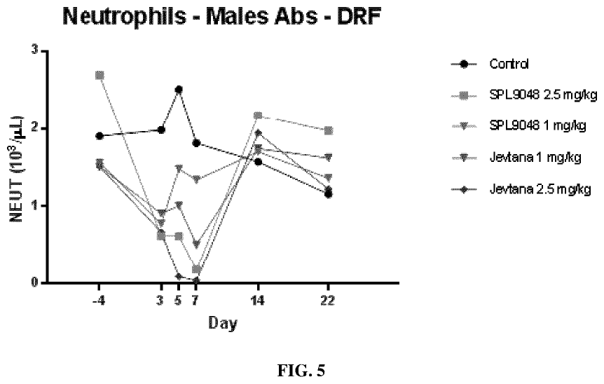

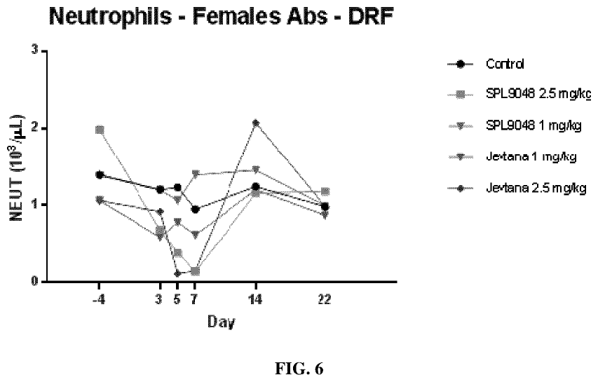

As shown in FIGS. 5 and 6, the results show that there is a separation in neutropenia at this dosage level (1 mg/kg) in both male and female rats, as evidenced by the dip in values seen with the administration of Jevtana® (i.e. cabazitaxel) and a lesser/no dip in values observed following administration of SPL9048 (see day 7 in particular). The rebound after day 7 appears to depend on the severity of neutropenia, as would be expected. In the 1 mg/kg Jevtana® (i.e., free cabazitaxel) groups, there is a substantial rebound at day 14, whereas there is virtually no rebound in the 1 mg/kg SPL9048 groups (or controls), which is consistent with limited neutropenia in these groups. This indicates that SPL9048 is likely to induce less neutropenia, and therefore be less toxic in the clinic, compared with the administration of an equivalent dose of free cabazitaxel.

Similar results were also found in a study at which SPL9048 and Jevtana® were delivered at 2.5 mg/kg active agent. SPL9048 was found to be less neutropenic at day 5 than Jevtana®. Reduced toxicity was observed for SPL9048 compared to Jevtana®/cabazitaxel. Test article related-hematology changes (decreases in white blood cells, neutrophils, lymphocytes, monocytes, eosinophil, platelets, and reticulocytes) were noted at 2.5 mg/kg SPL9048 and 2.5 mg/kg Jevtana® by Day 2 in males and females and remained low through Day 7. The decreases in these parameters were generally greater in rats administered 2.5 mg/kg Jevtana®.

Treatment-related microscopic changes were observed in the thymus, bone marrow, and spleen in animals administered SPL9048 at 2.5 mg/kg and Jevtana® at 2.5 mg/kg; the severity of the bone marrow and thymus findings was generally greater in Jevtana®-treated rats.

### Example 44: Linker Release Rates in PBS at 37° C. and pH 7.4

A study was carried out to determine the rate of cabazitaxel release from certain dendrimeric compounds in PBS (phosphate-buffered saline) at 37° C. and pH 7.4. The compounds tested were:
SPL9005, BHALys[Lys]₃₂[α-TDA-Cabazitaxel]_{32†}[ε-PEG 2100]_{32‡};
SPL9006, BHALys[Lys]₃₂[α-MIDA-Cabazitaxel]_{32†}[ε-PEG-2100]_{32‡};
SPL9048, BHALys[Lys]₃₂[α-DGA-Cabazitaxel]_{32†} [ε-PEG 2100]_{32‡}.
SPL9049, BHALys[Lys]₃₂[α-DGA-Cabazitaxel]_{32†} [ε-PEG_{˜1100}]_{32‡}.

Results indicating the % cabazitaxel released at 24 hours for two repeat experiments are shown in the table below, together with the mean time to 50% release (or estimated mean time to 50% release based on datapoints):

% of Cabazitaxel released in PBS at 37° C. and pH 7.4:

| | % API | % API | Time to |
|---|---|---|---|
| | released at | released at | 50% release |
| | 24 hours | 24 hours | (mean) |
| | (Exp #1) | (Exp #2) | (Exp #2) |
| SPL9005 | 11.9 | 15 | Estimated at 5-7 days |
| SPL9006 | 7.5 | 8 | Estimated at 6-8 days |
| SPL9048 | 37 | 41 | 36 hours |
| SPL9049 | 51.5 | 32 | 54 hours |

Data for SPL9048 and SPL9049 at additional timepoints in Exp #2 is also provided below:

% of Cabazitaxel released in PBS at 37° C. and pH 7.4:

| time (h) | SPL9049 | SPL9048 |
|---|---|---|
| 0 | 0.97 | 0.95 |
| 24 | 32.06 | 41.28 |
| 48 | 45.156 | 55.48 |
| 67 | 59.676 | 62.65 |
| 87 | 67.86 | 71.12 |

The results demonstrate the relative release rates of cabazitaxel from the dendrimer following administration. SPL9005 results in the release of about 12 to 15% cabazitaxel over 24 hours in PBS at 37° C. and pH 7.4, SPL9006 (MIDA linker) results in the release of about 8% cabazitaxel in PBS at 37° C. and pH 7.4 over the same time period, SPL9048 results in the release of about 40% cabazitaxel in PBS at 37° C. and pH 7.4 over a 24 hour period, and SPL9049 results in the release of about 30 to 50% cabazitaxel under the same conditions.

SPL9048 has also been observed to have increased stability in solution (e.g. with regard to precipitation) compared with SPL9049, which may be attributed to the conjugate containing a PEG₂₂₀₀ group rather than a PEG₁₁₀₀ group.

1. A macromolecule comprising:
i) a dendrimer comprising:
a core; and
2 to 6 generations of building units, the outermost generation of building units having surface amino groups wherein at least two different terminal groups are covalently attached to the surface amino groups of the dendrimer;
ii) a first terminal group which is a residue of a pharmaceutically active agent comprising a hydroxyl group; and
iii) a second terminal group which is a pharmacokinetic modifying agent that comprises a polyethylene glycol (PEG);
wherein the first terminal group is covalently attached to the surface amino group of the dendrimer through a diacid linker, the diacid linker comprising an alkyl chain which is interrupted by one or more oxygen, sulfur or nitrogen atoms, and wherein the diacid linker forms an ester bond with the hydroxyl group of the pharmaceutically active agent and an amide bond with the surface amino group, or a pharmaceutically acceptable salt thereof.

2. The macromolecule according to claim 1, wherein the diacid linker has the formula:
—C(O)—X—C(O)—
wherein X is —(CH₂)ₛ-A-(CH₂)ₜ—;
A is selected from —O—, —S— and —NR₁—;
R₁ is selected from hydrogen and C₁-C₄ alkyl; and
s and t are independently selected from 1 and 2.

3. The macromolecule according to claim 2, wherein X is —CH₂-A-CH₂— or —CH₂CH₂-A-CH₂CH₂—.

4. (canceled)

5. (canceled)

6. (canceled)

7. The macromolecule according to claim 1, wherein the dendrimer has 4 to 6 generations of building units.

8. The macromolecule according to claim 1, wherein the dendrimer has 5 generations of building units.

9. The macromolecule according to claim 1, wherein the building units are lysines.

10. The macromolecule according to claim 1, wherein the core is a benzhydrylyamide of lysine (BHALys).

11. (canceled)

12. (canceled)

13. (canceled)

14. The macromolecule according to claim 1, wherein the first terminal group and the second terminal group are present in a 1:1 ratio.

15. A pharmaceutical composition comprising the macromolecule of claim 1 and a pharmaceutically acceptable carrier.

16. (canceled)

17. (canceled)

18. A method of treating or suppressing the growth of a cancer in a subject, comprising administering an effective amount of a macromolecule according to claim 1 to the subject.

19. (canceled)

20. (canceled)

21. The macromolecule according to claim 1, wherein the polyethylene glycol has a molecular weight in the range of 220 to 1100 Da, or 1000 to 2500 Da, or 1000-5500 Da.

22. The macromolecule according to claim 1, wherein the diacid linker comprises an alkyl chain interrupted by one or more oxygen, sulfur or nitrogen atoms, or an aryl, cycloalkyl, heterocyclic or heteroaryl group.

23. The macromolecule according to claim 2, wherein A is selected from O—, —S—, —NH— and —N(CH₃)—.

24. The macromolecule according to claim 1, wherein the macromolecule comprises a blocking group, a pharmaceutical agent or a targeting group.

25. The macromolecule according to claim 1, comprising a targeting group attached to a functional group on the core of the dendrimer.

26. The macromolecule according to claim 25, wherein the targeting agent is selected from luteinising hormone releasing hormone, a luteinising hormone releasing hormone analogue, LYP-1 and an antibody or antibody fragment.

27. A macromolecule according to claim 1, wherein the pharmaceutically active agent is docetaxel, paclitaxel, cabazitaxel, camptothecin, irinotecan, topotecan or gemcitabine.
